(19) United States
(12) Patent Application Publication
(10) Pub. No.: US 2023/0372328 A1
(43) Pub. Date: Nov. 23, 2023

(54) ORAL DELAYED BURST FORMULATION OF LOW-DOSE NALTREXONE AND METHODS FOR TREATING FIBROMYALGIA AND LONG COVID

(71) Applicant: Scilex Holding Company, Palo Alto, CA (US)

(72) Inventors: Shanmuka Harish CHALAMURI, Cranbury, NJ (US); Ashok PERUMAL, Canbury, NJ (US); Yu-Hsing TU, Cranbury, NJ (US); Tien-Li LEE, San Diego, CA (US); Zhenhuan ZHENG, San Diego, CA (US)

(21) Appl. No.: 18/296,247

(22) Filed: Apr. 5, 2023

Related U.S. Application Data
(63) Continuation of application No. PCT/US21/53645, filed on Oct. 5, 2021.
(60) Provisional application No. 63/088,416, filed on Oct. 6, 2020.
(60) Provisional application No. 63/154,795, filed on Feb. 28, 2021.

(51) Int. Cl. A61K 31/485; A61K 9/48; A61K 9/20; A61K 9/28

(52) U.S. Cl. CPC A61K 31/485; A61K 9/4866; A61K 9/485; A61K 9/2018; A61K 9/2054; A61K 9/2013; A61K 9/2027; A61K 9/2009; A61K 9/2866; A61K 9/282; A61K 9/4891; A61K 9/2846; A61K 9/2086

(57) ABSTRACT

The present disclosure relates to oral delayed burst formulations comprising naltrexone or naloxone. The present disclosure also relates to doses, capsules, and tablets comprising the oral delayed burst formulation. The present disclosure also relates to methods of treating chronic pain, fibromyalgia, and long-Covid using the oral delayed burst formulation, doses, capsules, and tablets.

### TECHNICAL FIELD

The present disclosure relates to oral delayed burst formulations comprising naltrexone or naloxone. The present disclosure also relates to doses, capsules, and tablets comprising the oral delayed burst formulations. The present disclosure also relates to methods of treating chronic pain, fibromyalgia, and long-Covid using the oral delayed burst formulation, doses, capsules, and tablets.

### CROSS-REFERENCE TO RELATED APPLICATION

This application is a continuation of International Application No. PCT/US2021/053645, filed Oct. 5, 2021, which claims the benefit of U.S. Provisional Application No. 63/154,795, filed Feb. 28, 2021 and of U.S. Provisional Application No. 63/088,416, filed Oct. 6, 2020, the contents of which are incorporated by reference herein in their entireties.

### BACKGROUND

Naltrexone is an oral opioid receptor antagonist approved by the US Food and Drug Administration (FDA) in 1984 for opiate addiction and in 1994 for alcohol dependence at doses of 50-100 mg/day. Low-dose naltrexone (LDN is <5 mg/day) is not FDA-approved, but has been used off-label for a variety of chronic pain conditions. However, ingestion of opioid antagonists, including LDN, has immediate and undesirable side effects, including hyperalgesia, dysphoria, nausea, vomiting, anxiety, and insomnia (including delayed sleep onset) that are not well tolerated. Therefore, there is a need in the art to provide a LDN formulation that avoids side effects of opioid antagonists.

### Side Effects of Naltrexone:

Side effects of LDN, even at doses at little as 1.5 mg, have been reported. There are three categories of side effects: Very common (10% or more), common (1-10%), and uncommon (<1%). Very common side effects are headache, dizziness, insomnia, anxiety, nausea, vomiting, diarrhea, alanine aminotransferase increase, aspartate aminotransferase increase, joint stiffness, rashes, abnormal creatinine phosphokinase levels, and pharyngitis.

Common side effects are somnolence, sedation, depression, dry mouth, muscle cramps, and nasopharyngitis. Uncommon side effects are lethargy, cerebral arterial aneurysm, convulsions, disturbance in attention, dysgeusia, mental impairment, migraine, ischemic stroke, paresthesia, suicide attempt, ideation, abdominal discomfort, colitis, constipation, gastroesophageal reflux disease, gastrointestinal hemorrhage, hemorrhoids, pancreatitis acute, paralytic ileus, lymphadenopathy including cervical adenitis, white blood cell count increased, cholecystitis, cholelithiasis, chills, joint stiffness, muscle spasms, myalgia, pain in limb angina pectoris, angina unstable, atrial fibrillation, cardiac failure congestive, coronary artery atherosclerosis, myocardial infarction, palpitations, deep vein thrombosis, chronic obstructive pulmonary disease, dyspnea, pharyngolaryngeal pain, sinus congestion, dehydration, face edema, night sweats, pruritus, sweating, decreased platelet count, conjunctivitis, and blurred vision.

Naltrexone blocks endogenous opioid receptors, thus abrogating the effects of endogenous opioids such as enkephalins. This results in hyperalgesia, dysphoria, nausea, vomiting, anxiety, and insomnia (including delayed sleep onset), and other subjectively adverse experiences when conscious. Mechanism-wise, transient blockade of endogenous opioid receptors also elicits compensatory upregulation of opioid receptor expression and endogenous opioids (Tempel et al., J. Pharmacol. Exp. Ther. 232(2):439-444; and Zagon et al., (1995) Brain Res. Mol. Brain Res. 33(1): 111-120) which confers therapeutic benefit for a number of chronic pain conditions.

### Fibromyalgia

Fibromyalgia (FM) is a complex syndrome characterized by chronic widespread musculoskeletal pain which is often accompanied by multiple other symptoms, including fatigue, sleep disturbances, decreased physical functioning, and dyscognition. Due to these multiple symptoms, as well as high rates of comorbidity with other related disorders, patients with FM often report a reduced quality of life. Although the pathophysiology of FM is not completely understood, patients with FM experience pain differently from the general population, most likely due to dysfunctional pain processing in the central nervous system leading to both hyperalgesia and allodynia.

Most patients with fibromyalgia have a reduced “quality of life”, particularly as regards physical exertion, sleep, employment, sex life, vocational pursuits and maintaining friendships. Heretofore, there has been no generally accepted efficacious treatment of fibromyalgia. The best that could be achieved was to help patients learn to live with their problem through costly multidisciplinary treatment programs.

Managing acute pathology often relies on addressing the underlying pathology and symptoms of the disease. There is a need in the art for new compositions to treat fibromyalgia pain.

### Long-Covid

Most people who have coronavirus disease 2019 (COVID-19) recover completely within a few weeks. But some people, even those who had mild versions of the disease, continue to experience symptoms after their initial recovery. These people sometimes describe themselves as “long haulers” and the condition has been called post-COVID-19 syndrome or “long-Covid-19.”

Large numbers of patients who have been infected with SARS-CoV-2 continue to experience a constellation of symptoms long past the time that they've recovered from the initial stages of COVID-19 illness. Often referred to as “Long-Covid”, these symptoms (which can include fatigue, shortness of breath, “brain fog”, sleep disorders, fevers, gastrointestinal symptoms, anxiety, and depression) can persist for months and can range from mild to incapacitating. In some cases, new symptoms arise well after the time of infection or evolve over time. While still being defined, these effects can be collectively referred to as Post-Acute Sequelae of SARS-CoV-2 infection (PASC).

Older people and people with many serious medical conditions are the most likely to experience lingering COVID-19 symptoms, but even young, otherwise healthy people can feel unwell for weeks to months after infection. The most common signs and symptoms that linger over time include:
Fatigue
Shortness of breath
Cough
Joint pain
Chest pain

Other long-term signs and symptoms may include:
Muscle pain or headache
Fast or pounding heartbeat
Loss of smell or taste
Memory, concentration or sleep problems
Rash or hair loss

There is currently no treatment option for Long-Covid, other than traditional treatment approaches to whatever symptoms arise, such as more rest for fatigue. Therefore, there is a need in the art for a treatment option for Long-Covid regardless of the specific symptom cluster experienced.

### SUMMARY

The present disclosure provides to oral delayed burst formulations. In some embodiments, the oral delayed burst formulations comprise naltrexone. In some embodiments, the oral delayed release formulation releases low-dose naltrexone with a lag time after administration to an individual. Some embodiments relate to methods of treating chronic pain, fibromyalgia, or Long-Covid by orally administering the oral delayed burst formulation shortly before sleeping.

The present disclosure provides an oral delayed burst formulation comprising
(a) a core comprising naltrexone, or a pharmaceutically acceptable salt thereof, and at least one pharmaceutical excipient, and
(b) a delayed release layer, and
wherein the oral delayed burst formulation comprises between about 1 to about 5 mg of naltrexone, or a corresponding amount of a pharmaceutically acceptable salt thereof.

The present disclosure also provides an oral delayed burst formulation comprising
(a) a core comprising about 1 to about 5 mg of naltrexone, or a corresponding amount of a pharmaceutically acceptable salt thereof, and at least one pharmaceutical excipient,
(b) a subcoat layer, and
(c) a delayed release layer
wherein the oral delayed burst formulation comprises sucrose, hydroxypropyl methylcellulose, talc, cross-linked sodium carboxymethylcellulose, poly(methacrylic acid, ethyl acrylate) copolymer, and triethyl citrate.

The present disclosure further provides a dose of the oral delayed burst formulation described herein, wherein the dose comprises about 1 to about 5, about 1 to about 4.9, about 1 to about 4.5, about 1 to about 4, about 1 to about 3.5, about 1 to about 3, about 2 to about 5, about 2 to about 4.5, about 2 to about 4, about 2 to about 3.5, about 2 to about 3, about 3 to about 5, about 3 to about 4.9, about 3 to about 4.5, about 3 to about 4, or about 3 to about 3.5 mg of the naltrexone or a corresponding amount of the pharmaceutically acceptable salt thereof.

The present disclosure further provides a capsule or a table comprising the oral delayed burst formulation or the dose described herein.

The present disclosure also provides a method for treating chronic pain in a subject in need thereof, the method comprising orally administering the oral delayed burst formulation, the dose, or the capsule or tablet described herein to the subject shortly before sleep.

The present disclosure also provides a method for treating fibromyalgia in a subject in need thereof, the method comprising orally administering the oral delayed burst formulation, the dose, or the capsule or tablet described herein to the subject shortly before sleep.

The present disclosure also provides a method for treating long-Covid in a subject in need thereof, the method comprising orally administering the oral delayed burst formulation, the dose, or the capsule or tablet described herein to the subject shortly before sleep.

In another embodiment, the present disclosure also provides an oral delayed burst formulation comprising
(a) a core comprising naloxone, or a pharmaceutically acceptable salt thereof, and at least one pharmaceutical excipient, and
(b) a delayed release layer, and
wherein the oral delayed burst formulation comprises between about 10 mg to about 40 mg of naloxone, or a corresponding amount of a pharmaceutically acceptable salt thereof.

### BRIEF DESCRIPTION OF THE DRAWINGS

Various aspects of the present disclosure are illustrated by reference to the accompanying drawings.

FIG. 1 shows the results of dissolution tests in 2 stage media (0.1N HCl and pH 5.5 buffer) of 2 mg naltrexone HCl capsules with differing formulations.

FIG. 2 shows the results dissolution tests in 2 stage media (0.1N HCl and pH 5.5 buffer) of 2 mg naltrexone HCl capsules with varying amounts of enteric coating.

FIG. 3 shows the results dissolution tests in 2 stage media (0.1N HCl and pH 5.5 buffer) of 2 mg naltrexone HCl capsules with varying amounts of super disintegrant.

FIG. 4 shows a flowchart outlining a manufacturing process for naltrexone capsules.

FIG. 5 shows the results of a dissolution test in 2 stage media of a 2 mg naltrexone HCl capsule.

FIG. 6 shows the results of dissolution tests in 2 stage media (0.1N HCl and pH 5.5 or pH 6.8 buffer) of a 4.5 mg naltrexone HCl capsule.

### DETAILED DESCRIPTION

As used above, and throughout this disclosure, the following terms, unless otherwise indicated, shall be understood to have the following meanings. If a term is missing, the conventional term as known to one skilled in the art controls.

As used herein, the terms “including,” “containing,” and “comprising” are used in their open, non-limiting sense.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

To provide a more concise descriptions, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Concentrations that are given as percentages refer to mass ratios, unless indicated differently.

As used herein, except where specified as otherwise, “about” refers to a value within 10% of the value shown. For example, a mass of about 4 mg would also include values from 3.6 mg to 4.4 mg, unless specified otherwise.

“Pharmaceutically acceptable” includes molecular entities and compositions that do not produce adverse, allergic or other untoward reaction when administered to an animal, or a human, as appropriate. “Pharmaceutically acceptable salts” include acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. In a non-limiting example, naltrexone HCl is a pharmaceutically acceptable salt of naltrexone.

The term “delayed-release” refers to a medication that does not immediately disintegrate and release the active ingredient(s) into the body. The term “delayed-release” is used with reference to a drug formulation having a release profile in which there is a predetermined delay in the release of the drug following administration. The delayed-release formulation includes an enteric coating, which is a barrier applied to oral medication that prevents release of medication before it reaches the small intestine. Delayed-release formulations, such as enteric coatings, prevent drugs having an irritant effect on the stomach, such as aspirin, from dissolving in the stomach. Such coatings are also used to protect acid-unstable drugs from the stomach's acidic exposure, delivering them instead to a basic pH environment (intestine's pH 5.0 and above) where they do not degrade, and give their desired action.

Most enteric coatings work by presenting a surface that is stable at the highly acidic pH found in the stomach, but that breaks down rapidly at a less acidic (relatively more basic) pH. Therefore, an enteric coated pill will not dissolve in the acidic environment of the stomach (pH −3), but they will in the alkaline (pH 7-9) environment present in the small intestine.

The term “burst release” is a type of delayed-release, which is used with reference to a drug formulation that provides delayed and then rapid release of the drug within a short time period immediately after a predetermined lag period, thereby producing a pulsed plasma profile of the drug after drug administration. Formulations may be designed to provide a single burst release at a predetermined time following administration.

The concept of delayed burst allows a dosage form to not release in the stomach, avoiding potential side effects shortly after taking the oral formulation, and to have a burst release of the drug in the small intestine to achieve a drug's benefit about 1-2 hours after swallowing the pharmaceutical formulation. In this way, such a pharmaceutical formulation is swallowed shortly before the individual goes to sleep, allowing the API (active pharmaceutical ingredient) to achieve its beneficial effects and side effects when the individual is asleep. In this regard, a benefit of a delayed bust formulation is for API's whose side effects are disturbing to a conscious individual to the point where the side effects limit patient compliance with prescription dosage regimen.

In the oral delayed burst formulation, one or more barrier coatings may be applied to facilitate slow dissolution and concomitant release of drugs into the intestine. In some embodiments, the barrier coating contains one or more polymers encasing, surrounding, or forming a layer or membrane around the therapeutic composition or active core. The active agents are delivered in a formulation to provide delayed and burst release at a pre-determined time following administration. In a non-limiting example, the delay may be from about 1 hour to about 3 hours.

A burst-release composition may comprise 100% of the total dosage of a given active agent administered in a single unit dose. A burst-release formulation typically comprises a barrier coating that delays the release of the active ingredient(s). The barrier coating may consist of a variety of different materials, depending on the objective. In addition, a formulation may comprise a plurality of barrier coatings to facilitate release in a temporal manner

The coating may be a sugar coating, a film coating (e.g., based on hydroxypropyl methylcellulose, methylcellulose, methyl hydroxyethyl cellulose, hydroxypropylcellulose, carboxymethylcellulose, acrylate copolymers, polyethylene glycols and/or polyvinylpyrrolidone), or a coating based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, the formulation may additionally include a time delay material such as, for example, glyceryl monostearate or glyceryl distearate.

The delayed-release formulation includes an enteric coating comprised of one or more polymers facilitating release of active agents in proximal or distal regions of the gastrointestinal tract. As used herein, the term “enteric polymer coating” means the coating resists dissolution in the stomach, but dissolves or erodes in more distal regions of the gastrointestinal tract, such as the small intestine or colon. The enteric polymer coating comprises one or more polymers having a pH-dependent or pH-independent release profile. In some embodiments, the enteric polymer coating comprises one or more polymers having a pH-dependent release profile. An enteric polymer coating typically resists releases of the active agents until sometime after a gastric emptying lag period after administration.

The present disclosure relates to oral delayed burst formulations. In some embodiments, the oral delayed burst formulations comprise naltrexone. In some embodiments, the oral delayed release formulation releases low-dose naltrexone with a lag time after administration to an individual. Some embodiments relate to methods of treating chronic pain, fibromyalgia, or Long-Covid by orally administering the oral delayed burst formulation shortly before sleeping.

An embodiment of the present disclosure is directed to an oral delayed burst formulation comprising(a) a core comprising naltrexone, or a pharmaceutically acceptable salt thereof, and at least one pharmaceutical excipient, and (b) a delayed release layer, and wherein the oral delayed burst formulation comprises between about 1 to about 5 mg of naltrexone, or a corresponding amount of a pharmaceutically acceptable salt thereof.

The oral delayed burst formulation is orally administered from a variety of drug forms designed to provide delayed and burst release. Delayed oral dosage forms include, for example, tablets, capsules, caplets, and may also comprise a plurality of granules, beads, powders, or pellets that may or may not be encapsulated. Tablets and capsules represent convenient oral dosage forms, in which case solid pharmaceutical carriers are employed. In a non-limiting example, the oral delayed burst formulation is in the form of granules, and the granules are enclosed in a capsule or a tablet.

### Core

The oral delayed burst formulation includes a core including the therapeutic composition. In some embodiments, the therapeutic composition is naltrexone, or a corresponding amount of the pharmaceutically acceptable salt thereof.

In some embodiments, the core comprises about 1 to about 10, about 1 to about 8, about 1 to about 5, about 1 to about 4, about 1 to about 3, about 1 to about 2.8, about 1 to about 2.5, about 1 to about 2, about 2 to about 10, about 2 to about 8, about 2 to about 5, about 2 to about 4, about 2 to about 3, about 2 to about 2.8, or about 2 to about 2.5 wt % naltrexone, or a corresponding amount of the pharmaceutically acceptable salt thereof relative to the total weight of the core. In some embodiments, the core comprises about 1 to about 5 wt % naltrexone, or a corresponding amount of the pharmaceutically acceptable salt thereof relative to the total weight of the core.

In some embodiments, the core comprises about 2, about 2.1, about 2.2, about 2.3, about 2.4, about 2.5, about 2.6, about 2.7, about 2.8, about 2.9, about 3, about 4, or about 5 wt % naltrexone, or a corresponding amount of the pharmaceutically acceptable salt thereof relative to the total weight of the core. In some embodiments, the core comprises about 2.5 wt % naltrexone, or a corresponding amount of the pharmaceutically acceptable salt thereof relative to the total weight of the core.

The core optionally includes a burst controlling agent. In some embodiments, the burst controlling agent comprises a water insoluble polymer for controlling the rate of penetration of water into the core and raising the internal pressure (osmotic pressure) inside the core. Such a burst controlling agent is able to swell upon contact with liquid, e.g., bodily fluids. Examples of the water insoluble polymer include cross-linked polysaccharides, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid.

Examples of the cross-linked polysaccharide include but are not limited to insoluble metal salts or cross-linked derivatives of alginate, pectin, xanthan gum, guar gum, tragacanth gum, and locust bean gum, carrageenan, metal salts thereof, and covalently cross-linked derivatives thereof.

Examples of the modified cellulose include but are not limited to cross-linked derivatives of hydroxypropylcellulose, hydroxypropyl methylcellulose, hydroxyethyl cellulose, methylcellulose, carboxymethylcellulose, and metal salts of carboxymethylcellulose. In some embodiments, the water insoluble polymer is calcium pectinate, microcrystalline cellulose, or a combination thereof.

In some embodiments, the core comprises at least one water insoluble core excipient, wherein the water insoluble excipient is selected from the group consisting of microcrystalline cellulose, lactose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide, cross-linked polyacrylic acid, and combinations of the foregoing. In some embodiments, the water insoluble core excipient is talc.

In some embodiments, the core comprises about 0.1 to about 1, about 0.1 to about 0.8, about 0.1 to about 0.6, about 0.1 to about 0.5, about 0.1 to about 0.4, about 0.1 to about 0.3, about 0.1 to about 0.25, about 0.1 to about 0.2, about 0.15 to about 1, about 0.15 to about 0.8, about 0.15 to about 0.6, about 0.15 to about 0.5 about 0.15 to about 0.4, about 0.15 to about 0.3, about 0.15 to about 0.25, about 0.15 to about 0.2, about 0.2 to about 1, about 0.2 to about 0.8, about 0.2 to about 0.6, about 0.2 to about 0.5, about 0.2 to about 0.4, about 0.2 to about 0.3, about 0.2 to about 0.25, about 0.25 to about 1, about 0.25 to about 0.8, about 0.25 to about 0.6, about 0.25 to about 0.5, about 0.25 to about 0.4, or about 0.25 to about 0.3 wt % of the water insoluble core excipient relative to the total weight of the core. In some embodiments, the core comprises about 0.1 to about 0.5 wt % of the water insoluble core excipient relative to the total weight of the core.

In some embodiments, the core comprises about 0.1, about 0.15, about 0.2, about 0.25, about 0.3, about 0.35, about 0.4, or about 0.5 wt % of the water insoluble core excipient relative to the total weight of the core. In some embodiments, the core comprises about 0.2 wt % of the water insoluble core excipient relative to the total weight of the core.

In some embodiments, the core contains one or more of an absorption enhancer, a binder, a disintegrant, at least one other excipient, or a combination thereof.

Examples of a binder include but are not limited to Povidone (PVP: polyvinyl pyrrolidone), low molecular weight HPC (hydroxypropyl cellulose), low molecular weight HPMC (hydroxypropyl methylcellulose), low molecular weight carboxymethyl cellulose, ethylcellulose, gelatin, polyethylene oxide, acacia, dextrin, magnesium aluminum silicate, starch, and polymethacrylates. In some embodiments, the binder is Povidone (HPMC-E5 (5 mpas)).

In some embodiments, the core further comprises a hydrophilic core excipient selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose, carboxymethyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, polysaccharide, hydroxypropyl methylcellulose phthalate, polyvinyl acetate phthalate, cellulose acetate phthalate, hydroxypropyl methylcellulose acetate succinate, poly(methacrylic acid, methyl methacrylate) copolymer, poly(methacrylic acid, ethyl acrylate) copolymer, alginic acid, sodium alginate, and combinations of the foregoing. In some embodiments, the hydrophilic core excipient is hydroxypropyl methylcellulose.

In some embodiments, the core comprises about 0.1 to about 1, about 0.1 to about 0.8, about 0.1 to about 0.6, about 0.1 to about 0.5, about 0.1 to about 0.4, about 0.1 to about 0.3, about 0.1 to about 0.25, about 0.1 to about 0.2, about 0.15 to about 1, about 0.15 to about 0.8, about 0.15 to about 0.6, about 0.15 to about 0.5 about 0.15 to about 0.4, about 0.15 to about 0.3, about 0.15 to about 0.25, about 0.15 to about 0.2, about 0.2 to about 1, about 0.2 to about 0.8, about 0.2 to about 0.6, about 0.2 to about 0.5, about 0.2 to about 0.4, about 0.2 to about 0.3, about 0.2 to about 0.25, about 0.25 to about 1, about 0.25 to about 0.8, about 0.25 to about 0.6, about 0.25 to about 0.5, about 0.25 to about 0.4, or about 0.25 to about 0.3 wt % of the hydrophilic core excipient relative to the total weight of the core. In some embodiments, the core comprises about 0.1 to about 0.5 wt % of the hydrophilic core excipient relative to the total weight of the core.

In some embodiments, the core comprises about 0.1, about 1.5, about 0.2, about 0.25, about 0.3, about 3.5, about 0.4, about 0.45, or about 0.5 wt % of the hydrophilic core excipient relative to the total weight of the core. In some embodiments, the core comprises about 0.2 wt % of the hydrophilic core excipient relative to the total weight of the core.

Disintegrants may be added to the formulation to overcome the cohesive strength imparted during compression, thus facilitating breakup of the formulation in the body and increasing the surface area for dissolution. They can be either intragranular, extragranular, or both. On contact, disintegrants can draw water into the formulation, swelling and forcing it apart.

Examples of a disintegrant include but are not limited to, croscarmellose sodium (cross-linked sodium carboxymethyl cellulose), crospovidone (cross-linked PVP), sodium carboxymethyl starch (sodium starch glycolate), pregelatinized starch (starch 1500), microcrystalline starch, water insoluble starch, calcium carboxymethyl cellulose, magnesium aluminum silicate (Veegum) or a combination thereof. In some embodiments, the disintegrant is croscarmellose sodium.

Superdisintegrants represent a subclass of disintegrants that are associated with dramatic disintegration rates. Some of the common superdisintegrants are crospovidone, croscarmellose sodium, and sodium starch glycolate.

In some embodiments, the core further comprises at least one core disintegrant, wherein the core disintegrant is selected from the group consisting of polyvinylpyrrolidone, starch glycolate, starch, carboxymethylcellulose, hydroxypropylcellulose, magnesium aluminum silicate, and combinations of the foregoing. In some embodiments, the core disintegrant is selected from the group consisting of cross-linked polyvinylpyrrolidone, sodium starch glycolate, cross-linked sodium carboxymethylcellulose, pregelatinized starch, microcrystalline starch, water insoluble starch, calcium carboxymethylcellulose, low substituted carboxymethylcellulose, low substituted hydroxypropylcellulose, magnesium aluminum silicate, and combinations of the foregoing. In some embodiments, the core disintegrant is cross-linked sodium carboxymethylcellulose.

In some embodiments, the core comprises about 0.5 to about 5, about 0.5 to about 4, about 0.5 to about 3, about 0.5 to about 2, about 0.5 to about 1.5, about 0.5 to about 1.3, about 0.5 to about 1, about 0.8 to about 5, about 0.8 to about 4, about 0.8 to about 3, about 0.8 to about 2, about 0.8 to about 1.5, about 0.8 to about 1.3, about 0.8 to about 1, about 1 to about 5, about 1 to about 4, about 1 to about 3, about 1 to about 2, about 1 to about 1.5, or about 1 to about 1.3 wt % of the core disintegrant relative to the total weight of the core. In some embodiments, the core comprises about 0.5 to about 3 wt % of the core disintegrant relative to the total weight of the core.

In some embodiments, the core comprises about 1, about 1.1, about 1.2, about 1.3, about 1.4, or about 1.5 wt % of the core disintegrant relative to the total weight of the core. In some embodiments, the core comprises about 1 wt % of the core disintegrant relative to the total weight of the core.

In some embodiments, the water insoluble core excipient is a superdisintegrant.

In some embodiments, the core further comprises a synergist agent (sequestrate) and optionally a chelating agent.

In some embodiments, the sequestrate is selected from the group consisting of citric acid and ascorbic acid. In some embodiments, a sequestrate such as ascorbic acid has several hydroxyl and/or carboxylic acid groups, which can provide a supply of hydrogen for regeneration of an inactivated antioxidant free radical. A sequestrate therefore in some embodiments acts as a supplier of hydrogen for rejuvenation of the primary antioxidant.

In some embodiments, the chelating agent is selected from the group consisting of antioxidants, dipotassium edetate, disodium edetate, edetate calcium disodium, edetic acid, fumaric acid, malic acid, maltol, sodium edetate, trisodium edetate, and combinations of the foregoing. A chelating agent, such as citric acid, is intended to help in chelation of trace quantities of metals thereby assisting to prevent the loss of the active ingredient(s) by oxidation.

The core can further comprise an antioxidant. In some embodiments, the antioxidant is selected from the group consisting of 4,4 (2,3 dimethyl tetramethylene dipyrochatechol), tocopherol-rich extract (natural vitamin E), α-tocopherol (synthetic Vitamin E), β-tocopherol, γ-tocopherol, δ-tocopherol, butylhydroxinon, butyl hydroxyanisole (BHA), butyl hydroxytoluene (BHT), propyl gallate, octyl gallate, dodecyl gallate, tertiary butylhydroquinone (TBHQ), fumaric acid, malic acid, ascorbic acid (Vitamin C), sodium ascorbate, calcium ascorbate, potassium ascorbate, ascorbyl palmitate, ascorbyl stearate, citric acid, sodium lactate, potassium lactate, calcium lactate, magnesium lactate, anoxomer, erythorbic acid, sodium erythorbate, erythorbin acid, sodium erythorbin, ethoxyquin, glycine, gum guaiac, sodium citrates (monosodium citrate, disodium citrate, trisodium citrate), potassium citrates (monopotassium citrate, tripotassium citrate), lecithin, polyphosphate, tartaric acid, sodium tartrates (monosodium tartrate, disodium tartrate), potassium tartrates (monopotassium tartrate, dipotassium tartrate), sodium potassium tartrate, phosphoric acid, sodium phosphates (monosodium phosphate, disodium phosphate, trisodium phosphate), potassium phosphates (monopotassium phosphate, dipotassium phosphate, tripotassium phosphate), calcium disodium ethylene diamine tetra-acetate (calcium disodium EDTA), lactic acid, trihydroxy butyrophenone, thiodipropionic acid, and combinations thereof. In some embodiments, the antioxidant is BHA.

The core may further comprise a stabilizer. In some embodiments, the stabilizer is a basic substance which elevates the pH of an aqueous solution or dispersion of the formulation to at least about 6.8. Examples of such basic substances include antiacids such as magnesium aluminometasilicate, magnesium aluminosilicate, magnesium aluminate, dried aluminum hydroxide, synthetic hydrotalcite, synthetic aluminum silicate, magnesium carbonate, precipitated calcium carbonate, magnesium oxide, aluminum hydroxide, and sodium hydrogencarbonate, and combinations of the foregoing; and pH-regulator agents such as L-arginine, sodium phosphate, disodium hydrogen phosphate, sodium dihydrogenphosphate, potassium phosphate, dipotassium hydrogenphosphate, potassium dihydrogenphosphate, disodium citrate, sodium succinate, ammonium chloride, sodium benzoate, and combinations of the foregoing.

The basic substance can be selected from the group consisting of an inorganic hydrophilic or inorganic water-insoluble compounds.

Examples of inorganic hydrophilic basic substances include, but are not limited to, carbonate salt such as sodium or potassium carbonate, sodium bicarbonate, potassium hydrogen carbonate, phosphate salts selected from, e.g., anhydrous sodium, potassium or calcium dibasic phosphate, trisodium phosphate, alkali metal hydroxides (selected from sodium, potassium, or lithium hydroxide), and mixtures thereof. Sodium bicarbonate advantageously serves to neutralize acid groups in the composition in the presence of moisture that may adsorb onto particles of the composition during storage. The calcium carbonate exerts a buffering action in the stored composition, without apparent effect on drug release upon ingestion. It has further been discovered that the carbonate salts sufficiently stabilize the drug substance such that conventional water-based preparative techniques, e.g. trituration with water or wet granulation, can be utilized to prepare stabilized compositions. Examples of inorganic water-insoluble basic substance include, but are not limited to, suitable alkaline compounds capable of imparting the requisite basicity, including certain pharmaceutically acceptable inorganic compounds commonly employed in antiacid compositions e.g., magnesium oxide, magnesium hydroxide, magnesium carbonate, magnesium hydrogen carbonate, aluminum or calcium hydroxide or carbonate, composite aluminum-magnesium compounds (such as magnesium aluminum hydroxide, silicate compound such as magnesium aluminum silicate (Veegum F), magnesium aluminometasilicate (Nesulin FH2), magnesium aluminosilicate (Nisulin A)), pharmaceutically acceptable salts of phosphoric acid such as tribasic calcium phosphate, and combinations of the foregoing.

Other optional ingredients for the core include, but are not limited to, one or more of a filler, a flow regulating agent and a lubricant.

Examples of suitable fillers include but are not limited to microcrystalline cellulose (e.g., Avicel®), starch, lactitol, lactose, dibasic calcium phosphate or any other type of suitable inorganic calcium salt and sucrose, or a combination thereof. In some embodiments, the filler is lactose monohydrate. Avicel® microcrystalline cellulose (MCC) is a purified, partially depolymerized alphacellulose excipient made by acid hydrolysis of specialty wood pulp.

In some embodiments, the core further comprises a core filler selected from the group consisting of microcrystalline cellulose, starch, lactitol, lactose, inorganic calcium salt, sucrose, and combinations of the foregoing. In some embodiments, the core filler is sucrose.

In some embodiments, the core comprises about 80 to about 99, about 80 to about 97, about 80 to about 96, about 80 to about 95, about 80 to about 90, about 85 to about 99, about 85 to about 97, about 85 to about 96, about 85 to about 95, about 85 to about 90, about 90 to about 99, about 90 to about 97, about 90 to about 96, about 90 to about 95, about 93 to about 99, about 93 to about 97, about 93 to about 96, or about 93 to about 95 wt % of the core filler relative to the total weight of the core. In some embodiments, the core comprises about 90 to about 99 wt % of the core filler relative to the total weight of the core.

In some embodiments, the core comprises about 90, about 95, about 96, or about 97 wt % of the core filler relative to the total weight of the core. In some embodiments, the core comprises about 96 wt % of the core filler relative to the total weight of the core.

In some embodiments, the naltrexone or pharmaceutically acceptable salt thereof is in a layer in contact with the core filler.

Examples of suitable lubricants include but are not limited to stearate salts such as magnesium stearate, calcium stearate, and sodium stearate; stearic acid; talc; sodium stearyl fumarate, and compritol (glycerol behenate), corola oil, glyceryl palmito stearate, hydrogenated vegetable oil, magnesium oxide, mineral oil, poloxamer, polyethylene glycol, polyvinyl alcohol, sodium benzoate, talc, sodium stearyl fumarate, compritol (glycerol behenate) and sodium lauryl sulfate (SLS) or a combination thereof. In some embodiments, the lubricant is magnesium stearate.

Examples of suitable flow regulating agents include but are not limited to colloidal silicon dioxide and aluminum silicate. In some embodiments, the flow regulating agent is colloidal silicon dioxide.

The core can also optionally include a buffering agent such as, for example, an inorganic salt compound and an organic alkaline salt compound. In some embodiments, the buffering agent is selected from the group consisting of potassium bicarbonate, potassium citrate, potassium hydroxide, sodium bicarbonate, sodium citrate, sodium hydroxide, calcium carbonate, dibasic sodium phosphate, monosodium glutamate, tribasic calcium phosphate, monoethanolamine, diethanolamine, triethanolamine, citric acid monohydrate, lactic acid, propionic acid, tartaric acid, fumaric acid, malic acid, and monobasic sodium phosphate.

According to specific embodiments, the core further includes a stabilizer. In some embodiments, the stabilizer comprises at least one of butyl hydroxyanisole, ascorbic acid and citric acid. A hardness enhancing agent is microcrystalline cellulose.

Optionally, the core further comprises a preservative. In some embodiments, the preservative is selected from the group consisting of antioxidants, dipotassium edetate, disodium edetate, edetate calcium disodium, edetic acid, fumaric acid, malic acid, maltol, sodium edetate, and trisodium edetate.

In some embodiments, the core comprises combinations of the ingredients disclosed herein. For example, in some embodiments the core comprises cross-linked sodium carboxymethylcellulose, sucrose, and the naltrexone or the pharmaceutically acceptable salt thereof. In some embodiments, the core comprises about 1 wt % sodium carboxymethylcellulose, about 95 wt % sucrose, and about 2.5 wt % naltrexone or a corresponding amount of the pharmaceutically acceptable salt thereof relative to the total weight of the core. In some embodiments, the core further comprises hydroxypropyl methylcellulose and talc. In some embodiments, the core comprises about 0.2% hydroxypropyl methylcellulose and about 0.2 wt % talc relative to the total weight of the core.

### Subcoat Layer

In some embodiments, a subcoat layer (e.g., a sealing coat) is applied. A subcoat layer protects the core from absorbing moisture and hardens the surface to provide protection for the core in subsequent process steps. The subcoat layer may modify the rate of release of naltrexone and may prevent premature release of naltrexone from the core.

In some embodiments, the oral delayed burst formulation comprises (c) a subcoat layer, wherein the subcoat layer comprises at least one hydrophilic subcoat excipient. In some embodiments, the subcoat layer is between the core and the delayed release layer.

In some embodiments, the oral delayed burst formulation comprises about 90 to about 99, about 90 to about 98, about 90 to about 97, about 90 to about 96.5, about 90 to about 96, about 90 to about 95, about 92 to about 99, about 92 to about 98, about 92 to about 97, about 92 to about 96.5, about 92 to about 96, about 92 to about 95, about 94 to about 99, about 94 to about 98, about 94 to about 97, about 94 to about 96.5, about 94 to about 96, about 94 to about 95, about 95 to about 99, about 95 to about 98, about 95 to about 97, about 95 to about 96.5, about 95 to about 96, about 96 to about 97, about 96 to about 96.6, or about 96 to about 96.5 wt % of the core relative to the total weight of the core and the subcoat layer. In some embodiments, the oral delayed burst formulation comprises about 95 to about 99 wt % of the core relative to the total weight of the core and the subcoat layer.

In some embodiments, the oral delayed burst formulation comprises about 95, about 96, about 96.5, about 96.6, or about 97 wt % of the core relative to the total weight of the core and the subcoat layer. In some embodiments, the oral delayed burst formulation comprises about 97 wt % of the core relative to the total weight of the core and the subcoat layer.

In some embodiments, the hydrophilic subcoat excipient is a hydrophilic carrier. In some embodiments, the hydrophilic subcoat excipient is selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose HPMC, carboxy methyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, water soluble gum, polysaccharide, hydroxypropyl methylcellulose phthalate, polyvinyl acetate phthalate, cellulose acetate phthalate, hydroxypropyl methylcellulose acetate succinate, poly(methacrylic acid, methyl methacrylate)1:1, poly(methacrylic acid, ethyl acrylate)1:1, alginic acid, and sodium alginate, and any other pharmaceutically acceptable polymer that dissolves in phosphate buffer pH>5.0 or mixtures thereof. In some embodiments, the hydrophilic carrier is polyvinyl pyrrolidone.

In some embodiments, the hydrophilic subcoat excipient is selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose, carboxymethyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, polysaccharide, hydroxypropyl methylcellulose phthalate, polyvinyl acetate phthalate, cellulose acetate phthalate, hydroxypropyl methylcellulose acetate succinate, poly(methacrylic acid, methyl methacrylate) copolymer, poly(methacrylic acid, ethyl acrylate) copolymer, alginic acid, sodium alginate, and combinations of the foregoing. In some embodiments, the hydrophilic subcoat excipient is hydroxypropyl methylcellulose.

In some embodiments, the subcoat layer comprises about 1 to about 10, about 1 to about 8, about 1 to about 6, about 1 to about 5, about 1 to about 4, about 1 to about 3, about 1 to about 2.5, about 1 to about 2, about 1.5 to about 10, about 1.5 to about 8, about 1.5 to about 6, about 1.5 to about 5, about 1.5 to about 4, about 1.5 to about 3, about 1.5 to about 2.5, about 1.5 to about 2, about 2 to about 10, about 2 to about 8, about 2 to about 6, about 2 to about 5, about 2 to about 4, about 2 to about 3, about 2 to about 2.5, about 2.5 to about 1, about 2.5 to about 8, about 2.5 to about 6, about 2.5 to about 5, about 2.5 to about 4, or about 2.5 to about 3 wt % of the hydrophilic subcoat excipient relative to the total weight of the core and the subcoat layer. In some embodiments, the subcoat layer comprises about 1 to about 5 wt of the hydrophilic subcoat excipient relative to the vital weight of the core and the subcoat layer.

In some embodiments, the subcoat layer comprises about 1, about 2, about 2.5, about 3, about 3.5, about 4, about 4.5, or about 5 wt % of the hydrophilic subcoat excipient relative to the total weight of the core and the subcoat layer. In some embodiments, the subcoat layer comprises about 3 wt % of the hydrophilic subcoat excipient relative to the total weight of the core and the subcoat layer.

In some embodiments, the subcoat further comprises at least one water insoluble particulate matter, wherein the water insoluble particulate matter is selected from the group consisting of microcrystalline cellulose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide and cross-linked polyacrylic acid. In some embodiments, the water insoluble particulate matter is microcrystalline cellulose. In some embodiments, the hydrophilic carrier of the subcoat is a combination of povidone and microcrystalline cellulose. In some embodiments, the water insoluble particular matter is a water insoluble subcoat excipient.

In some embodiments, the subcoat layer further comprises at least one water insoluble subcoat excipient, wherein the water insoluble subcoat excipient is selected from the group consisting of microcrystalline cellulose, lactose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide, cross-linked polyacrylic acid, and combinations of the foregoing. In some embodiments, the subcoat layer comprises talc.

In some embodiments, the subcoat layer comprises about 0.5 to about 2, about 0.5 to about 1.5, about 0.5 to about 1, about 0.5 to about 0.9, about 0.5 to about 0.8, about 0.7 to about 2, about 0.7 to about 1.5, about 0.7 to about 1, about 0.7 to about 0.9, about 0.7 to about 0.8, or about 0.8 to about 0.9 wt % of the water insoluble subcoat excipient relative to the total weight of the core and the subcoat layer. In some embodiments, the subcoat layer comprises about 0.5 to about 1 wt % of the water insoluble subcoat excipient relative to the total weight of the core and the subcoat layer.

In some embodiments, the subcoat layer comprises about 0.7, about 0.8, about 0.9 wt %, or about 1 wt % of the water insoluble subcoat excipient relative to the total weight of the core and the subcoat layer. In some embodiments, the subcoat layer comprises about 1 wt % of the water insoluble subcoat excipient relative to the total weight of the core and the subcoat layer.

In some embodiments, the subcoat layer comprises combinations of the ingredients disclosed herein. For example, in some embodiments the subcoat layer comprises hydroxypropyl methylcellulose and talc. In some embodiments, the subcoat layer comprises about 2.5 wt % hydroxypropyl methylcellulose and about 0.8 wt % talc relative to the total weight of the core and the subcoat layer.

### Delayed Release Layer

The oral delayed release formulation includes a delayed release layer (e.g., an enteric coating) for facilitating release of active agents in proximal or distal regions of the gastrointestinal tract.

In some embodiments, the oral delayed burst formulation comprises about 20 to about 40, about 20 to about 35, about 20 about 33, about 20 to about 32, about 20 to about 30, about 25 to about 40, about 25 to about 35, about 25 to about 33, about 25 to about 32, about 28 to about 40, about 28 to about 35, about 28 about 33, about 28 to about 32, about 30 to about 40, about 30 to about 35, about 30 about 33, or about 30 to about 32 wt % of the delayed release layer relative to the total weight of the oral delayed burst formulation. In some embodiments, the oral delayed burst formulation comprises about 25 to about 35 wt % of the delayed release layer relative to the total weight, of the oral delayed burst formulation.

In some embodiments, the oral delayed burst formulation comprises about 30, about 31, about 32, about 33, about 34, or about 35 wt % of the delayed release layer relative to the total weight of the oral delayed burst formulation. In some embodiments, the oral delayed burst formulation comprises about 32 wt % of the delayed release layer relative to the total weight of the oral delayed burst formulation.

The enteric coating can comprise any suitable enteric coating material, such as hydroxypropyl methylcellulose phthalate, polyvinyl acetate phthalate, cellulose acetate phthalate, hydroxypropyl methylcellulose acetate succinate, poly(methacrylic acid, methyl methacrylate)1:1 (EUDRAGIT® L 100), poly(methacrylic acid, ethyl acrylate)1:1 (EUDRAGIT® L 30 D-55), (EUDRAGIT® L 100-55), shellac, alginic acid, and sodium alginate. In some embodiments, the delayed release layer is an enteric coating. In some embodiments, the delayed release polymer is an enteric coating material.

In some embodiments, the delayed release polymer is a pH dependent polymer, sometimes referred to as a pH dependent enteric coating polymer. In some embodiments, the pH dependent polymer resists dissolution in the acidic medium of the stomach, but dissolves or erodes in more distal regions of the gastrointestinal tract, such as the small intestine or colon.

In some embodiments, the delayed release layer further comprises one or more delayed release polymers selected from the group consisting of hydroxypropyl methylcellulose phthalate, polyvinyl acetate phthalate, cellulose acetate phthalate, hydroxypropyl methylcellulose acetate succinate, poly(methacrylic acid, methyl methacrylate)copolymer, poly(methacrylic acid, ethyl acrylate) copolymer, alginic acid, sodium alginate, and combinations of the foregoing.

In some embodiments, the delayed release polymer is poly(methacrylic acid, ethyl acrylate) copolymer. In some embodiments, the delayed release polymer is poly(methacrylic acid, ethyl acrylate)1:1 copolymer. In some embodiments, the delayed release polymer is an aqueous dispersion. In some embodiments, the aqueous dispersion comprises sodium lauryl sulfate and polysorbate. In some embodiments, the aqueous dispersion comprises about 0.7% sodium lauryl sulfate and 2.3% polysorbate. In some embodiments, the delayed release polymer is EUDRAGIT® L 30 D-55.

EUDRAGIT® L 30 D-55 is an aqueous dispersion of an anionic copolymer based on methacrylic acid and ethyl acrylate. The ratio of the free carboxyl groups to the ester groups is approx. 1:1. The dispersion contains 0.7% sodium lauryl sulfate and 2.3% polysorbate 80, as emulsifiers.

In some embodiments, the delayed release layer comprises about 10 to about 30, about 10 to about 25, about 10 to about 23, about 10 to about 22, about 10 to about 20, about 15 to about 30, about 15 to about 25, about 15 to about 23, about 15 to about 22, about 15 to about 20, about 18 to about 30, about 18 to about 25, about 18 to about 23, about 18 to about 22, about 18 to about 20, about 20 to about 30, about 20 to about 25, about 20 to about 23, or about 20 to about 22 wt % of the delayed release polymer relative to the total weight of the oral delayed burst formulation. In some embodiments, the delayed release layer comprises about 15 to about 25 wt % of the delayed release polymer relative to the total weight of the oral delayed burst formulation.

In some embodiments, the delayed release layer comprises about 15, about 20, or about 25 wt % of the delayed release polymer relative to the total weight of the oral delayed burst formulation. In some embodiments, the delayed release layer comprises about 20 wt % of the delayed release polymer relative to the total weight of the oral delayed burst formulation.

The delayed release layer may further a water soluble excipient.

In some embodiments, the delayed release layer further comprises at least one water insoluble delayed release layer excipient, wherein the water insoluble delayed release layer excipient is selected from the group consisting of microcrystalline cellulose, lactose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide, cross-linked polyacrylic acid, and combinations of the foregoing. In some embodiments, the water insoluble delayed release layer excipient is talc.

In some embodiments, the delayed release layer comprises about 1 to about 20, about 1 to about 15, about 1 to about 12, about 1 to about 10, about 5 to about 20, about 5 to about 15, about 5 to about 12, about 5 to about 10, about 8 to about 20, about 8 to about 15, about 8 to about 12, or about 8 to about 10 wt % of the water insoluble delayed release layer excipient relative to the total weight of the oral delayed burst formulation. In some embodiments, the delayed release layer comprises about 5 to about 15 wt % of the water insoluble delayed release layer excipient relative to the total weight of the oral delayed burst formulation.

In some embodiments, the delayed release layer comprises about 5, about 8, about 10, about 12, about 15, or about 20 wt % of the water insoluble delayed release layer excipient relative to the total weight of the oral delayed burst formulation. In some embodiments, the delayed release layer comprises about 10 wt % of the water insoluble delayed release layer excipient relative to the total weight of the oral delayed burst formulation.

The outer enteric coating may further comprise a plasticizer. In some embodiments, the plasticizer includes at least one of dibutyl sebacate, polyethylene glycol, polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof. In some embodiments, the delayed release coating is an outer enteric coating and the plasticizer is a delayed release layer plasticizer.

In some embodiments, the delayed release layer further comprises one or more delayed release layer plasticizers selected from the group consisting of triethyl citrate, acetyl triethyl citrate, acetyltributyl citrate, polyethylene glycol acetylated monoglycerides, glycerin, triacetin, propylene glycol, phthalate esters, titanium dioxide, ferric oxides, castor oil, sorbitol, dibutyl sebacate, and combinations of the foregoing. In some embodiments, the delayed release layer comprises triethyl citrate.

In some embodiments, the delayed release layer comprises about 0.2 to about 10, about 0.2 to about 5, about 0.2 to about 4, about 0.2 to about 3, about 0.2 to about 2.5, about 0.2 to about 2, about 1 to about 10, about 1 to about 5, about 1 to about 4, about 1 to about 3, about 1 to about 2.5, about 1 to about 2, about 1.5 to about 10, about 1.5 to about 5, about 1.5 to about 4, about 1.5 to about 3, about 1.5 to about 2.5, or about 1.5 to about 2 wt % of the delayed release layer plasticizer relative to the total weight of the oral delayed burst formulation. In some embodiments, the delayed release layer comprises about 1 to about 5 wt % of the delayed release layer plasticizer relative to the total weight of the oral delayed burst formulation.

In some embodiments, the delayed release layer comprises about 1, about 1.5, about 2, or about 2.5 wt % of delayed release layer plasticizer relative to the total weight of the oral delayed burst formulation. In some embodiments, the delayed release layer comprises about 2 wt % of the delayed release layer plasticizer relative to the total weight of the oral delayed burst formulation.

The delayed release layer may further comprise anti-tackiness agents, such as talc or glyceryl monostearate.

The delayed release layer may further comprise one or more plasticizers including, but not limited to, triethyl citrate, acetyl triethyl citrate, acetyltributyl citrate, polyethylene glycol acetylated monoglycerides, glycerin, triacetin, propylene glycol, phthalate esters (e.g., diethyl phthalate, dibutyl phthalate), titanium dioxide, ferric oxides, castor oil, sorbitol, and dibutyl sebacate.

In some embodiments, the enteric coating comprises methacrylic acid copolymer, triethyl citrate and talc. Such methods include a wet granulation process, a dry mix process, a direct compression process, etc. In one embodiment, several of the core ingredients are mixed by a wet granulation process to form a granulate, which is then dried and dry-mixed with several other ingredients to form the core, which is then coated.

In some embodiments, the delayed release layer comprises combinations of the ingredients disclosed herein. For example, in some embodiments the delayed release layer comprises poly(methacrylic acid, ethyl acrylate) copolymer and triethyl citrate. In some embodiments, the delayed release layer comprises about 20 wt % poly(methacrylic acid, ethyl acrylate) copolymer and about 2 wt % triethyl citrate relative to the total weight of oral delayed burst formulation. In some embodiments, the delayed release layer further comprises talc. In some embodiments, the delayed release layer comprises about 10 wt % talc relative to the total weight of oral delayed burst formulation.

In another embodiment, capsules or bilayered tablets may be formulated to contain a drug-containing core, covered by a swelling layer, and an outer insoluble but semi-permeable polymer coating or membrane. The lag time prior to rupture can be controlled by the permeation and mechanical properties of the polymer coating and the swelling behavior of the swelling layer. In some embodiments, the swelling layer comprises one or more swelling agents, such as swellable hydrophilic polymers that swell and retain water in their structures.

Exemplary water swellable materials include polyethylene oxide (having e.g., an average molecular weight between 1,000,000 to 7,000,000, such as POLYOX®); methylcellulose; hydroxypropyl cellulose; hydroxypropyl methylcellulose; polyalkylene oxides having a weight average molecular weight of 100,000 to 6,000,000, including but not limited to poly(methylene oxide) and poly(butylene oxide); poly(hydroxy alkyl methacrylate) having a molecular weight of from 25,000 to 5,000,000; poly(vinyl)alcohol having a low acetal residue, which is cross-linked with glyoxal, formaldehyde or glutaraldehyde and having a degree of polymerization of from 200 to 30,000; mixtures of methyl cellulose, cross-linked agar and carboxymethyl cellulose; hydrogel forming copolymers produced by forming a dispersion of a finely divided copolymer of maleic anhydride with styrene, ethylene, propylene, butylene or isobutylene cross-linked with from 0.001 to 0.5 moles of saturated cross-linking agent per mole of maleic anhydride in the copolymer; CARBOPOL® acidic carboxy polymers having a molecular weight of 450,000 to 4,000,000; CYANAMER® polyacrylamides; cross-linked water swellable indene-maleic anhydride polymers; GOODRITE® polyacrylic acid having a molecular weight of 80,000 to 200,000; starch graft copolymers; AQUAKEEPS® acrylate polymer polysaccharides composed of condensed glucose units such as diester cross-linked polyglucan; carbomers having a viscosity of 3,000 to 60,000 mPa as a 0.5%-1% w/v aqueous solution; cellulose ethers such as hydroxypropylcellulose having a viscosity of about 1000-7000 mPa·s as a 1% w/w aqueous solution (25° C.); hydroxypropyl methylcellulose having a viscosity of about 1000 or higher, in some embodiments 2,500 or higher to a maximum of 25,000 mPa as a 2% w/v aqueous solution; polyvinylpyrrolidone having a viscosity of about 300-700 mPa s as a 10% w/v aqueous solution at 20° C.; and combinations of the foregoing.

In some embodiments, a coating surrounding the drug (LDN) containing core comprises water-insoluble hydrophilic particulate matter embedded in a water-insoluble hydrophobic carrier. In some embodiments, the outer coating is not pH sensitive. In some embodiments, the water-insoluble hydrophobic carrier is a water insoluble polymer.

Examples of suitable hydrophobic carriers include but are not limited to dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, said polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes.

In some embodiments, the water-insoluble, hydrophilic particulate matter in the outer coating is a water insoluble but permeable polymer. Examples of such polymers include a water insoluble cross-linked polysaccharide, a water insoluble cross-linked protein, a water insoluble cross-linked peptide, water insoluble cross-linked gelatin, water insoluble cross-linked hydrolyzed gelatin, water insoluble cross-linked collagen, water insoluble cross linked polyacrylic acid, water insoluble cross-linked cellulose derivatives, water insoluble cross-linked polyvinyl pyrrolidone, micro crystalline cellulose, insoluble starch, micro crystalline starch, and combinations thereof. In some embodiments, the water insoluble particulate matter is micro crystalline cellulose. In some embodiments, the water-insoluble hydrophilic particulate matter comprises a mixture of Avicel® (microcrystalline cellulose) and ethocel.

The outer coating can optionally include at least one plasticizer. Examples of suitable plasticizers include, but are not limited to, cetyl alcohol, dibutyl phthalate, diethyl phthalate, dibutyl sebacate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol, polyethylene glycol, propylene glycol, sorbitol, and combinations thereof. In some embodiments, the amount of plasticizer is in a range of from about 0 to about 50% weight per weight of the water insoluble polymer in the film coat. In addition or alternatively, a stiffening agent such as cetyl alcohol can also be used.

The outer coating or the core or both can also optionally contain at least one of a wetting agent, suspending agent, surfactant, dispersing agent, or combinations thereof.

Examples of suitable wetting agents include, but are not limited to, poloxamer, polyoxyethylene ethers, polyoxyethylene sorbitan fatty acid esters (polysorbates), polyoxymethylene stearate, sodium lauryl sulfate, sorbitan fatty acid esters, benzalkonium chloride, polyethoxylated castor oil, docusate sodium, and combinations thereof.

Examples of suitable suspending agents include, but are not limited to, alginic acid, bentonite, carbomer, carboxymethylcellulose, carboxymethylcellulose calcium, hydroxyethyl cellulose, hydroxypropyl cellulose, microcrystalline cellulose, colloidal silicon dioxide, dextrin, gelatin, guar gum, xanthan gum, kaolin, magnesium aluminum silicate, maltitol, medium chain triglycerides, methylcellulose, polyoxyethylene sorbitan fatty acid esters (polysorbates), polyvinyl pyrrolidone (PVP), propylene glycol alginate, sodium alginate, sorbitan fatty acid esters, tragacanth, and combinations thereof.

Examples of suitable surfactants include anionic surfactants such as docusate sodium and sodium lauryl sulfate; cationic surfactants such as cetrimide; and nonionic surfactants such as polyoxyethylene sorbitan fatty acid esters (polysorbates) and sorbitan fatty acid esters.

Examples of suitable dispersing agents include but are not limited to, poloxamer, polyoxyethylene sorbitan fatty acid esters (polysorbates), sorbitan fatty acid esters, and combinations thereof. The content of the wetting agent, surfactant, dispersing agent and suspending agent can range in an amount of from about 0 to about 30% of the weight of the film coat of the formulation.

In some embodiments, the coating includes crospovidone (cross-linked PVP) or croscarmellose, calcium pectinate, microcrystalline cellulose, ethylcellulose, polyvinyl pyrrolidone (PVP), colloidal silicon dioxide, butyl hydroxyanisole, citric acid, ascorbic acid, and magnesium stearate. In some embodiments, the coating includes ethyl cellulose, cetyl alcohol, microcrystalline cellulose or calcium pectinate (CaP).

In some embodiments, the burst release formulation employs a water-permeable but insoluble film coating to enclose the active ingredient and an osmotic agent utilizing an enclosing. As water from the gut slowly diffuses through the film into the core, the core swells until the film bursts, thereby releasing the active ingredients. The film coating may be adjusted to permit various rates of water permeation or release time.

### Oral Delayed Burst Formulation

The oral delayed burst formulation includes combinations of the layers disclosed herein and their respective ingredients. For example, the oral delayed burst formulation may include the core and the delayed release layer and any of their respective ingredients. Also for example, the oral delayed burst formulation may include the core, the subcoating layer, and the delayed release layer and any of their respective ingredients.

For example, in some embodiments, the core comprises cross-linked sodium carboxymethylcellulose and the delayed release layer comprises poly(methacrylic acid, ethyl acrylate) copolymer.

In some embodiments, the core comprises cross-linked sodium carboxymethylcellulose and sucrose, the subcoat comprises hydroxypropyl methylcellulose, and the delayed release layer comprises poly(methacrylic acid, ethyl acrylate) copolymer.

In some embodiments, the oral delayed burst formulation comprises sucrose, hydroxypropyl methylcellulose, talc, cross-linked sodium carboxymethylcellulose, poly(methacrylic acid, ethyl acrylate) copolymer, and triethyl citrate.

In some embodiments, the oral delayed burst formulation comprises about 62.9 wt % sucrose, about 1.7 wt % naltrexone or a corresponding amount of the pharmaceutically acceptable salt thereof, about 1.9 wt % hydroxypropyl methylcellulose, about 10.7 wt % talc, about 0.8 wt % cross-linked sodium carboxymethylcellulose, about 20 wt % poly(methacrylic acid, ethyl acrylate) copolymer, and about 2.0 wt % triethyl citrate.

In one embodiment, an oral delayed burst formulation includes (a) a core comprising about 1 to about 5 mg of naltrexone, or a pharmaceutically acceptable salt thereof, and at least one pharmaceutical excipient, (b) a subcoat layer, and (c) a delayed release layer, wherein the oral delayed burst formulation comprises sucrose, hydroxypropyl methylcellulose, talc, cross-linked sodium carboxymethylcellulose, poly(methacrylic acid, ethyl acrylate) copolymer, and triethyl citrate.

In some embodiments, the oral delayed burst formulation comprises about 50 to about 70 wt % sucrose, about 0.5 to about 3 wt % hydroxypropyl methylcellulose, about 7 to about 15 wt % talc, about 0.4 to about 1.5 wt % cross-linked sodium carboxymethylcellulose, about 15 to about 25 wt % poly(methacrylic acid, ethyl acrylate) copolymer, and about 1 to about 4 wt % triethyl citrate relative to the total weight of the oral delayed burst formulation.

In some embodiments, the core comprises sucrose, hydroxypropyl methylcellulose, talc, and cross-linked sodium carboxymethylcellulose; the subcoat layer comprises hydroxypropyl methylcellulose, and talc; the delayed release layer comprises poly(methacrylic acid, ethyl acrylate) copolymer, talc, and triethyl citrate.

In some embodiments, the core comprises about 80 to about 97 wt % sucrose, about 0.1 to about 0.4 wt % hydroxypropyl methylcellulose, about 0.1 to about 0.4 wt % talc, and about 0.5 to about 3 wt % cross-linked sodium carboxymethylcellulose relative to the total weight of the core; the subcoat layer comprises about 1 to about 4 wt % hydroxypropyl methylcellulose, and about 0.3 to about 2 wt % talc relative to the total weight of the core and subcoat layer; and the delayed release layer comprises about 10 to about 30 wt % poly(methacrylic acid, ethyl acrylate) copolymer, about 5 to about 15 wt % talc, and about 0.5 to about 4 wt % triethyl citrate relative to the total weight of the oral delayed burst formulation.

### Naltrexone

Naltrexone can be produced from noroxymorphone by various direct and indirect alkylation methods. One method is by direct alkylation of noroxymorphone with cyclopropylmethylbromide. This process has been disclosed in WO 91/05768, the disclosure of which is incorporated by reference herein. WO 2008/034973, the disclosure of which is incorporated by reference herein, describes a process for obtaining naltrexone in 88.6% yield by reacting noroxymorphone hydrochloride with cyclopropylmethylbromide in dimethylacetamide in the presence of sodium hydrogen carbonate. WO 2008/138605, the disclosure of which is incorporated by reference herein, describes N-alkylation of noroxymorphone with cyclopropylmethylbromide in N-methyl-pyrrolidone in the presence of sodium hydrogen carbonate. WO 2010/039209, the disclosure of which is incorporated by reference herein, describes N-alkylation of noroxymorphone with cyclopropylmethylbromide in the presence of a protic solvent. Specific examples in WO 2010/039209 describe the addition of water, isopropanol or ethanol as the protic solvent.

Naltrexone may be synthesized by producing naltrexone[17-(cyclopropylmethyl)-4,5α-epoxy-3,14-dihydroxy-morphinan-6-one] from noroxymorphone[4,5-α-epoxy-3,14-dihydroxy-morphinan-6-one] by alkylation with a cyclopropylmethyl halide.

The concept of “delay and burst” is to enable the dosage form not to release in the stomach avoiding the potential side effects, but to have immediate burst of the drug release achieving the maximum benefit of its effectiveness. This is helpful for bedtime administration, avoiding immediate side effects while achieving maximum effectiveness.

Naltrexone is incorporated into cores with or without disintegrant, either by extrusion/spheronization and/or drug layering process, followed by a film coating with a pH-dependent release controlling polymer. The pH-dependent release controlling polymer will protect the drug from being released in the acidic environments of the stomach; once the oral delayed burst formulations move to higher pH (around pH 5.0), the film will start to dissolve/erode allowing the penetration of water to enable the core to explode with or without the aid of disintegrant resulting in a burst release of naltrexone.

Throughout the present disclosure, amounts of naltrexone disclosed refer to the amount of naltrexone free form present in the formulation. The term “corresponding amount” as used herein refers to the amount of a pharmaceutically acceptable salt of naltrexone required to obtain the amount of naltrexone free form recited in the formulation. It would be clear to one of skill in the art how to calculate the “corresponding amount” of the salt of a compound, such as the corresponding amount of the pharmaceutically acceptable salt of naltrexone, taking into account the difference in molecular weight between the free form of a compound and a salt form. For example, 4.1 mg of naltrexone free base would correspond to 4.5 mg of the HCl salt.

In some embodiments, the oral delayed burst formulation comprises about 1 to about 5, about 1 to about 4.9, about 1 to about 4.5, about 1 to about 4, about 1 to about 3.5, about 1 to about 3, about 2 to about 5, about 2 to about 4.5, about 2 to about 4, about 2 to about 3.5, about 2 to about 3, about 3 to about 5, about 3 to about 4.9, about 3 to about 4.5, about 3 to about 4, or about 3 to about 3.5 mg of the naltrexone or a corresponding amount of the pharmaceutically acceptable salt thereof. In some embodiments, the oral delayed burst formulation comprises about 2 to about 4.5 mg of the naltrexone or a corresponding amount of the pharmaceutically acceptable salt thereof.

In some embodiments, the oral delayed burst formulation comprises about 2, about 2.5, about 3, about 3.5, about 4, or about 4.5 mg of the naltrexone or a corresponding amount of the pharmaceutically acceptable salt thereof. In some embodiments, the oral delayed burst formulation comprises about 2 mg of the naltrexone or a corresponding amount of the pharmaceutically acceptable salt thereof. In some embodiments, the oral delayed burst formulation comprises about 4 mg of the naltrexone or a corresponding amount of the pharmaceutically acceptable salt thereof.

In some embodiments, the pharmaceutically acceptable salt is naltrexone HCl. In some embodiments, the oral delayed burst formulation comprises naltrexone HCl.

### Naloxone

Naloxone was approved by FDA in 1971 and first marketed as Narcan® injection for the complete or partial reversal of opioid intoxication. It has subsequently become a multi-source prescription generic drug and is manufactured by International Medication Systems, Limited (IMS) and Hospira, Inc. The injection is available in two strengths, 0.4 mg/mL and 1.0 mg/mL. Naloxone injection is approved worldwide and is on the WHO Model list of Essential Medicines as a specific antidote.

Naloxone hydrochloride is a synthetic congener of oxymorphone. In structure it differs from oxymorphone in that the methyl group on the nitrogen atom is replaced by an allyl group. It is known chemically as 17-allyl-4,5 α-epoxy, 3-14-dihydroxymorphinan-6-one hydrochloride. It has a molecular weight of 363.84 for the anhydrate HCl salt and a molecular weight of 399.9 for the dihydrate HCl salt. Naloxone hydrochloride dihydrate has the structural formula:

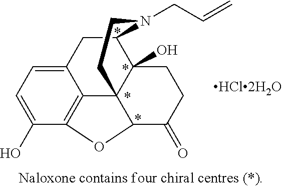

Naloxone hydrochloride occurs as a white to slightly off-white powder, and is soluble in water, in dilute acids, and in strong alkali. It is slightly soluble in alcohol, and practically insoluble in ether and in chloroform. Naloxone prevents or reverses the effects of opioids including respiratory depression, sedation and hypotension. Also, it can reverse the psychotomimetic and dysphoric effects of agonist-antagonists such as pentazocine. When administered in usual doses in the absence of opioids or agonistic effects of other opioid antagonists, it exhibits essentially no pharmacologic activity. Naloxone has not been shown to produce tolerance or cause physical or psychological dependence. In the presence of physical dependence on opioids, naloxone will produce withdrawal symptoms. However, in the presence of opioid dependence, withdrawal symptoms will appear within minutes of naloxone administration and will subside in about 2 hours. The severity and duration of the withdrawal syndromes are related to the dose and route of administration of naloxone and to the degree and type of dependence.

Throughout the present disclosure, amounts of naloxone disclosed refer to the amount of naloxone free form present in the formulation. The term “corresponding amount” as used herein refers to the amount of a pharmaceutically acceptable salt of naloxone required to obtain the amount of naloxone free form recited in the formulation. It would be clear to one of skill in the art how to calculate the “corresponding amount” of the salt of a compound, such as the corresponding amount of the pharmaceutically acceptable salt of naloxone, taking into account the difference in molecular weight between the free form of a compound and a salt form. For example, 9.00 mg of naloxone free base would correspond to 10.0 mg of naloxone HCl anhydrate salt.

An embodiment of the present disclosure is directed to an oral delayed burst formulation comprising (a) a core comprising naloxone, or a pharmaceutically acceptable salt thereof, and at least one pharmaceutical excipient, and (b) a delayed release layer, and wherein the oral delayed burst formulation comprises between about 10 mg to about 40 mg of naloxone, or a corresponding amount of a pharmaceutically acceptable salt thereof.

### Dosage

The present disclosure provides a delayed burst naltrexone formulation having from about a 1-4.5 mg daily dose for an adult human. In some embodiments, this formulation is designed to be taken within one hour of going to sleep, such that the delayed burst release of naltrexone is released in the GI tract after the patient is asleep and the Cmax pharmacokinetic systemic concentration is achieved while the patient is still asleep and before waking time. In some embodiments, the burst release of the naltrexone is achieved within 1 to 3 hours after oral dosing.

The reason for the pharmacokinetic parameters of the present disclosure is to make sure the level of naltrexone in the morning is below a threshold that starts to elicit paradoxical analgesia. At low enough doses, naltrexone actually conveys an analgesia effect.

Some embodiments are directed to a dose of the oral delayed burst formulation disclosed herein. In a non-limiting example, the dose may comprise a plurality of naltrexone pellets consisting of the oral delayed burst formulation. In some embodiments, the dose comprises about 1 to about 5, about 1 to about 4.9, about 1 to about 4.5, about 1 to about 4, about 1 to about 3.5, about 1 to about 3, about 2 to about 5, about 2 to about 4.5, about 2 to about 4, about 2 to about 3.5, about 2 to about 3, about 3 to about 5, about 3 to about 4.9, about 3 to about 4.5, about 3 to about 4, or about 3 to about 3.5 mg of the naltrexone or a corresponding amount of the pharmaceutically acceptable salt thereof. In some embodiments, the dose comprises about 2 to about 4.5 mg of the naltrexone or a corresponding amount of the pharmaceutically acceptable salt thereof.

In some embodiments, the dose comprises about 2, about 2.5, about 3, about 3.5, about 4, or about 4.5 mg of the naltrexone or a corresponding amount of the pharmaceutically acceptable salt thereof. In some embodiments, the dose comprises about 2 mg of the naltrexone or a corresponding amount of the pharmaceutically acceptable salt thereof. In some embodiments, the dose comprises about 4 mg of the naltrexone or a corresponding amount of the pharmaceutically acceptable salt thereof.

In some embodiments, the pharmaceutically acceptable salt is naltrexone HCl. In some embodiments, the dose comprises naltrexone HCl.

Alternatively, the present disclosure provides a delayed burst naloxone formulation having from about 10-40 mg daily dose for an adult human. This formulation has a higher dose than naltrexone because naloxone has a lower systemic bioavailability than naltrexone when administered orally. Naloxone and naltrexone are structurally similar and are both potent opioid antagonists. Naloxone is usually injected whereas naltrexone is given orally—naloxone has poor oral bioavailability (˜3%) and has a much shorter half-life.

As oral naltrexone has an oral bioavailability of 5-40%, one can achieve the same intended effect of delayed burst release of 1 mg-4.5 mg of naltrexone with an oral naloxone equivalent. But naloxone has a much shorter half-life (3 hours) to reduce system drug concentration effectively by the time the patient awakens.

### Dosage Forms

The pharmaceutical composition is orally administered from a variety of drug formulations designed to provide delayed and burst release. Delayed oral dosage forms include, for example, tablets, capsules, caplets, and may also comprise a plurality of granules, beads, powders, or pellets that may or may not be encapsulated. Tablets and capsules represent convenient oral dosage forms, in which case solid pharmaceutical carriers are employed.

The oral delayed burst formulation or the dose may be loaded into a capsule or a tablet. In a non-limiting example, a capsule may be loaded a plurality of pellets consisting of the oral delayed burst formulation. By including more or less of the oral delayed burst formulation within the capsule (e.g. by including more or fewer pellets within the capsule) the amount of active ingredient delivered by the capsule may be adjusted without altering the composition of the oral delayed burst formulation.

An embodiment of the present disclosure is directed to a capsule or a tablet including the oral delayed burst formulation or the dose described herein. In some embodiments, the capsule is a gelatin capsule.

In some embodiments, the capsule or tablet comprises about 1 to about 5, about 1 to about 4.9, about 1 to about 4.5, about 1 to about 4, about 1 to about 3.5, about 1 to about 3, about 2 to about 5, about 2 to about 4.5, about 2 to about 4, about 2 to about 3.5, about 2 to about 3, about 3 to about 5, about 3 to about 4.9, about 3 to about 4.5, about 3 to about 4, or about 3 to about 3.5 mg of the naltrexone or a corresponding amount of the pharmaceutically acceptable salt thereof. In some embodiments, the capsule or tablet comprises about 2 to about 4.5 mg of the naltrexone or a corresponding amount of the pharmaceutically acceptable salt thereof.

In some embodiments, the capsule or tablet comprises about 2, about 2.5, about 3, about 3.5, about 4, or about 4.5 mg of the naltrexone or a corresponding amount of the pharmaceutically acceptable salt thereof. In some embodiments, the capsule or tablet comprises about 2 mg of the naltrexone or a corresponding amount of the pharmaceutically acceptable salt thereof. In some embodiments, the capsule or tablet comprises about 4 mg of the naltrexone or a corresponding amount of the pharmaceutically acceptable salt thereof.

In some embodiments, the pharmaceutically acceptable salt is naltrexone HCl. In some embodiments, the capsule or the tablet comprises naltrexone HCl.

### Release

The in vivo dissolution profile may be approximated by a dissolution test comprising an acid stage (representing the stomach) and a buffer stage (representing the small intestine or colon.) The oral delayed burst formulation, the dose, or the capsule or tablet described herein has low release of the active ingredient in the acid stage and high release of the active ingredient in the buffer stage. The protocol for a suitable dissolution test is provided in Example 21. Other standard dissolution tests are well-known in the art, such as those described in U.S. Pharmacopeia, Chapter 711-Dissolution (January 2006).

In some embodiments, the oral delayed burst formulation, dose, capsule, or tablet releases at most about 1%, about 5%, about 10%, about 15%, or about 20% of the naltrexone or the pharmaceutically acceptable salt thereof in an acid stage as measured by a dissolution test.

In some embodiments, the oral delayed burst formulation, dose, capsule, or tablet releases at least about 50%, at least about 55%, at least about 60%, at least about 65%, at least about 70%, at least about 75%, at least about 80%, at least about 85%, at least about 90%, at least about 99%, at least about 99.9%, or about 100% of the naltrexone or the pharmaceutically acceptable salt thereof in a buffer stage as measured by a dissolution test.

In some embodiments, the oral delayed burst formulation, dose, capsule, or tablet releases at most about 1%, about 5%, about 10%, about 15%, or about 20% of the naltrexone or the pharmaceutically acceptable salt thereof in an acid stage as measured by a dissolution test and releases at least about 50%, at least about 55%, at least about 60%, at least about 65%, at least about 70%, at least about 75%, at least about 80%, at least about 85%, at least about 90%, at least about 99%, at least about 99.9%, or about 100% of the naltrexone or the pharmaceutically acceptable salt thereof in a buffer stage as measured by the dissolution test.

In some embodiments, the dissolution test is carried out in 750 mL of 0.1 N HCl at 37±0.5° C. for the first two hours and in 1000 mL of pH 5.5 buffer solution at 37±0.5° C. for the subsequent 80 minutes, and is performed in a USP Apparatus II (Paddle) with a rotational speed of 50 rpm.

In some embodiments, the dissolution test is carried out in 750 mL of 0.1 N HCl at 37±0.5° C. for the first two hours and in 1000 mL of pH 6.8 buffer solution at 37±0.5° C. for the subsequent 80 minutes, and is performed in a USP Apparatus II (Paddle) with a rotational speed of 50 rpm.

In some embodiments, the oral delayed burst formulation, the dose, or the capsule or tablet releases at most about 1%, about 5%, about 10%, about 15%, or about 20% of the naltrexone or the pharmaceutically acceptable salt thereof after exposure to 0.1N HCl for 120 minutes.

In some embodiments, the capsule or tablet of any one of claims 107 to 120, wherein the oral delayed burst formulation, the dose, or the capsule or tablet releases at most about 10% of the naltrexone or the pharmaceutically acceptable salt thereof after exposure to 0.1N HCl for 120 minutes.

In some embodiments, the oral delayed burst formulation, the dose, or the capsule or tablet releases at least about 50%, about 55%, about 60%, about 65%, or about 70% of the naltrexone or the pharmaceutically acceptable salt thereof after exposure to a pH 5.5 solution for 15 minutes.

In some embodiments, the capsule or tablet of any one of claims 107 to 122, wherein the oral delayed burst formulation, the dose, or the capsule or tablet releases at least about 75%, about 80%, about 85%, or about 90% of the naltrexone or the pharmaceutically acceptable salt thereof after exposure to a pH 5.5 solution for 30 minutes.

In some embodiments, the oral delayed burst formulation, the dose, or the capsule or tablet releases at least about 80%, about 85%, about 90%, about 95%, about 99%, about 99.9%, or about 100% of the naltrexone or the pharmaceutically acceptable salt thereof after exposure to a pH 5.5 solution for 45 minutes.

In some embodiments, the oral delayed burst formulation, the dose, or the capsule or tablet releases at least about 80%, about 85%, about 90%, about 95%, about 99%, about 99.9%, or about 100% of the naltrexone or the pharmaceutically acceptable salt thereof after exposure to a pH 5.5 solution for 80 minutes.

In some embodiments, the oral delayed burst formulation, the dose, or the capsule or tablet releases at most about 1%, about 5%, about 10%, about 15%, or about 20% of the naltrexone or the pharmaceutically acceptable salt thereof after exposure to 0.1N HCl for 120 minutes, and releases at least about 80%, about 85%, about 90%, about 95%, about 99%, about 99.9%, or about 100% of the naltrexone or the pharmaceutically acceptable salt thereof after exposure to a pH 5.5 solution for 15, 30, 45, or 80 minutes.

In some embodiments, the pH 5.5 solution is a citrate buffer.

In some embodiments, the oral delayed burst formulation, the dose, or the capsule or tablet releases at least about 80%, about 85%, about 90%, about 93%, about 95%, about 99%, about 99.9%, or about 100% the naltrexone or the pharmaceutically acceptable salt thereof after exposure to a pH 6.8 solution for 10 minutes.

In some embodiments, the oral delayed burst formulation, the dose, or the capsule or tablet releases at least about 80%, about 85%, about 90%, about 93%, about 95%, about 99%, about 99.9%, or about 100% of the naltrexone or the pharmaceutically acceptable salt thereof after exposure to a pH 6.8 solution for 30 minutes.

In some embodiments, the oral delayed burst formulation, the dose, or the capsule or tablet releases at least about 80%, about 85%, about 90%, about 93%, about 95%, about 99%, about 99.9%, or about 100% of the naltrexone or the pharmaceutically acceptable salt thereof after exposure to a pH 6.8 solution for 30 minutes.

In some embodiments, the oral delayed burst formulation, the dose, or the capsule or tablet releases at least about 80%, about 85%, about 90%, about 95%, about 99%, about 99.9%, or about 100% of the naltrexone or the pharmaceutically acceptable salt thereof after exposure to a pH 6.8 solution for 45 minutes.

In some embodiments, the oral delayed burst formulation, the dose, or the capsule or tablet releases at most about 1%, about 5%, about 10%, about 15%, or about 20% of the naltrexone or the pharmaceutically acceptable salt thereof after exposure to 0.1N HCl for 120 minutes, and releases at least about 80%, about 85%, about 90%, about 95%, about 99%, about 99.9%, or about 100% of the naltrexone or the pharmaceutically acceptable salt thereof after exposure to a pH 6.8 solution for 10, 20, 30, or 45 minutes.

In some embodiments, the pH 6.8 solution is a phosphate buffer.

The oral delayed burst formulation, the dose, or the capsule or tablet described herein facilitate release of active agents in proximal or distal regions of the gastrointestinal tract. In some embodiments, they resist dissolution in the acidic medium of the stomach, but dissolve or erode in more distal, higher pH regions of the gastrointestinal tract, such as the small intestine or colon.

In some embodiments, the oral delayed burst formulation, dose, capsule, or tablet releases at least about 75%, about 80%, about 85%, about 90%, about 99%, about 99.9%, or about 100% of the naltrexone or the pharmaceutically acceptable salt thereof about 1.5 to about 5, about 1.5 to about 4, about 1.5 to about 3.5, about 1.5 to about 3, about 1.5 to about 2.5, about 2 to about 5, about 2 to about 4, about 2 to about 3.5, about 2 to about 3, about 2 to about 2.5, about 2.5 to about 5, about 2.5 to about 4, about 2.5 to about 3.5, or about 2.5 to about 3 hours after administration to an individual in need thereof.

In some embodiments, the oral delayed burst formulation, dose, capsule, or tablet releases at least about 80% of the naltrexone or the pharmaceutically acceptable salt thereof about 1.5 to about 3 hours after administration to an individual in need thereof.

In some embodiments, the oral delayed burst formulation, dose, capsule, or tablet releases at least about 75%, about 80%, about 85%, about 90%, about 99%, about 99.9%, or about 100% of the naltrexone or the pharmaceutically acceptable salt thereof about 1.5, about 2, about 2.5, about 3, about 3.5, or about 4 hours after administration to an individual in need thereof.

In some embodiments, the oral delayed burst formulation, dose, capsule, or tablet releases at least about 90% of the naltrexone or the pharmaceutically acceptable salt thereof about 3 hours after administration to an individual in need thereof.

In some embodiments, the oral delayed burst formulation, dose, capsule, or tablet releases at most about 10%, about 5%, about 4%, about 3%, about 2%, about 1%, or about 0% of the naltrexone or the pharmaceutically acceptable salt thereof about 0.5 to about 2, about 0.5 to about 1.5, about 0.5 to about 1, about 1 to about 2, or about 1 to about 1.5 hours after administration to an individual in need thereof.

In some embodiments, the oral delayed burst formulation, dose, capsule, or tablet releases at most about 10% of the naltrexone or the pharmaceutically acceptable salt thereof about 0.5 to about 2 hours after administration to an individual in need thereof.

In some embodiments, the oral delayed burst formulation, dose, capsule, or tablet releases at most about 10%, about 5%, about 4%, about 3%, about 2%, about 1%, or about 0% of the naltrexone or the pharmaceutically acceptable salt thereof about 0.5, about 1, about 1.5, or about 2 hours after administration to an individual in need thereof.

In some embodiments, the oral delayed burst formulation, dose, capsule, or tablet releases at most about 10% of the naltrexone or the pharmaceutically acceptable salt thereof about 2 hours after administration to an individual in need thereof.

In some embodiments, the oral delayed burst formulation, dose, capsule, or tablet releases at most about 10%, about 5%, about 4%, about 3%, about 2%, about 1%, or about 0% of the naltrexone or the pharmaceutically acceptable salt thereof about 0.5 to about 2, about 0.5 to about 1.5, about 0.5 to about 1, about 1 to about 2, or about 1 to about 1.5 hours after administration to an individual in need thereof, and releases at least about 75%, about 80%, about 85%, about 90%, about 99%, about 99.9%, or about 100% of the naltrexone or the pharmaceutically acceptable salt thereof about 1.5 to about 5, about 1.5 to about 4, about 1.5 to about 3.5, about 1.5 to about 3, about 1.5 to about 2.5, about 2 to about 5, about 2 to about 4, about 2 to about 3.5, about 2 to about 3, about 2 to about 2.5, about 2.5 to about 5, about 2.5 to about 4, about 2.5 to about 3.5, or about 2.5 to about 3 hours after the administration to the individual.

In some embodiments, the oral delayed burst formulation, dose, capsule, or tablet releases at most about 10% of the naltrexone or the pharmaceutically acceptable salt thereof about 1 to about 2 hours after administration to an individual in need thereof, and releases at least about 80% the naltrexone or the pharmaceutically acceptable salt thereof about 2 to about 3 hours after the administration to the individual.

In some embodiments, the oral delayed burst formulation, dose, capsule, or tablet releases at most about 10%, about 5%, about 4%, about 3%, about 2%, about 1%, or about 0% of the naltrexone or the pharmaceutically acceptable salt thereof about 0.5, about 1, about 1.5, or about 2 hours after administration to an individual in need thereof, and releases at least about 75%, about 80%, about 85%, about 90%, about 99%, about 99.9%, or about 100% of the naltrexone or the pharmaceutically acceptable salt thereof about 1.5, about 2, about 2.5, about 3, about 3.5, or about 4 hours after the administration to the individual.

In some embodiments, the oral delayed burst formulation, dose, capsule, or tablet releases at most about 10% of the naltrexone or the pharmaceutically acceptable salt thereof about 2 hours after administration to an individual in need thereof, and releases at least about 90% the naltrexone or the pharmaceutically acceptable salt thereof about 3 hours after the administration to the individual.

### Methods of Treatment

The disclosed oral delayed burst formulations, doses, capsules and tablets have activity as pharmaceuticals, as discussed herein.

The present disclosure provides a method of treating chronic pain in a subject in need thereof, the method comprising orally administering the oral delayed burst formulation, dose, capsule or tablet as described herein to the subject shortly before sleep.

In some embodiments, the subject has fibromyalgia, central sensitization syndrome, chronic regional pain syndrome, opioid dependence, endogenous opioid dysregulation, axial lower back pain, multiple sclerosis, Crohn's disease, diabetic neuropathy, long-Covid, or combinations of the foregoing.

The present disclosure also provides a method of treating fibromyalgia in a subject in need thereof, the method comprising orally administering the oral delayed burst formulation, dose, capsule or tablet as described herein to the subject shortly before sleep.

The present disclosure also provides a method of treating long-Covid in a subject in need thereof, the method comprising orally administering the oral delayed burst formulation, dose, capsule or tablet as described herein to the subject shortly before sleep.

In some embodiments, the subject in need thereof previously had Covid-19.

In some embodiments, the oral delay burst formulation is orally administered less than about 2, about 1.5, about 1, or about 0.5 hours before sleep.

In some embodiments, the administration of the oral delayed burst formulation results in a reduced frequency or severity of one or more side effects in the subject in need thereof as compared to the one or more side effects from administration of an immediate release form of naltrexone or pharmaceutically acceptable salt thereof.

In some embodiments, the administration of a single dose of the oral delayed burst formulation results in a reduced frequency or severity of one or more side effects in the subject in need thereof as compared to the one or more side effects from administration of the same dose of an immediate release form of naltrexone or a corresponding amount of a pharmaceutically acceptable salt thereof.

The one or more side effects is selected from the list consisting of headache, dizziness, insomnia, anxiety, nausea, vomiting, diarrhea, alanine aminotransferase increase, aspartate aminotransferase increase, joint stiffness, rashes, abnormal creatinine phosphokinase levels, pharyngitis, somnolence, sedation, depression, dry mouth, muscle cramps, nasopharyngitis, lethargy, cerebral arterial aneurysm, convulsions, disturbance in attention, dysgeusia, mental impairment, migraine, ischemic stroke, paresthesia, suicide attempt, ideation, abdominal discomfort, colitis, constipation, gastroesophageal reflux disease, gastrointestinal hemorrhage, hemorrhoids, acute pancreatitis, paralytic ileus, lymphadenopathy including cervical adenitis, increased white blood cell count, cholecystitis, cholelithiasis, chills, joint stiffness, muscle spasms, myalgia, pain in limb, angina pectoris, unstable angina, atrial fibrillation, congestive cardiac failure, coronary artery atherosclerosis, myocardial infarction, palpitations, deep vein thrombosis, chronic obstructive pulmonary disease, dyspnea, pharyngolaryngeal pain, sinus congestion, dehydration, face edema, night sweats, pruritus, sweating, decreased platelet count, conjunctivitis, blurred vision, and combinations of the foregoing,

In some embodiments, the one or more side effects is selected from the list consisting of headache, dizziness, insomnia, anxiety, nausea, vomiting, diarrhea, alanine aminotransferase increase, aspartate aminotransferase increase, joint stiffness, rashes, abnormal creatinine phosphokinase levels, pharyngitis, somnolence, sedation, depression, dry mouth, muscle cramps, nasopharyngitis, and combinations of the foregoing.

In some embodiments, the one or more side effects is selected from the list consisting of headache, dizziness, insomnia, anxiety, nausea, vomiting, diarrhea, alanine aminotransferase increase, aspartate aminotransferase increase, joint stiffness, rashes, abnormal creatinine phosphokinase levels, pharyngitis, and combinations of the foregoing.

### Description of a Formulation Embodiment

In some embodiments, naltrexone HCl delayed-release capsules or tablets are drug-layered naltrexone HCl granules (cores), coated with a delayed-release membrane and encapsulated into a hard gelatin capsule. The formulation design is intended to release all of the dose at a pH of around 5.0 and above as one burst. Naltrexone hydrochloride is solubilized in a suitable solvent along with a binder and other excipient and layered to sugar spheres using a fluid bed processor equipped with a Wurster or a rotor insert or produced as cores using an extruder and spheronizer. The granules are then dried to remove residual solvents to acceptable limits. An additional coating may be applied to the cores followed by delayed release polymer coating using a fluid bed processor equipped with a Wurster or a rotor insert.

In one embodiment, the present disclosure provides a delayed-release coating comprising a water insoluble capsule body closed at one end with an insoluble, but permeable and swellable hydrogel plug, wherein the plug comprises a material selected from the group consisting of polymethacrylates, erodible compressed polymers, congealed melted polymer and enzymatically controlled erodible polymers.

In another embodiment, the present disclosure provides a delayed burst release oral formulation for delayed burst release of a low-dose naltrexone (DBR-LDN) or a pharmaceutically acceptable salt or ester thereof in the gastrointestinal tract of a subject, comprising:
(a) a core comprising low-dose naltrexone (LDN), and at least one burst controlling agent, wherein the burst controlling agent is a water insoluble polymer;
(b) a subcoat surrounding the core comprising at least one water soluble hydrophilic carrier; and
(c) an outer enteric delayed release coating over the core, the outer coating comprising a water insoluble hydrophobic carrier and a water insoluble hydrophilic particulate matter, the water insoluble hydrophilic particulate matter allowing entry of liquid into the core.

In some embodiments, the outer enteric delayed release coating comprises a combination of at least one swellable polymer and at least one water insoluble polymer. In some embodiments, the outer coating is a two-layered coating comprising a rupturing outer layer and swellable inner layer. In some embodiments, the outer coaling further comprises a surfactant. In some embodiments, the surfactant in the outer coating is sodium lauryl sulfate (SLS).

In some embodiments, the hydrophilic carrier of the subcoat is selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose HPMC, carboxy methyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, water soluble gum, polysaccharide, hydroxypropyl methylcellulose phthalate, polyvinyl acetate phthalate, cellulose acetate phthalate, hydroxypropyl methylcellulose acetate succinate, poly(methacrylic acid, methyl methacrylate) 1:1, poly(methacrylic acid, ethyl acrylate)1:1, alginic acid, and sodium alginate, and any other pharmaceutically acceptable polymer that dissolves in phosphate buffer pH>5.0 or mixtures thereof. In some embodiments, the hydrophilic carrier is polyvinyl pyrrolidone. In some embodiments, the subcoat further comprises at least one water insoluble particulate matter, wherein the water insoluble particulate matter is selected from the group consisting of microcrystalline cellulose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide and cross-linked polyacrylic acid. In some embodiments, the water insoluble particulate matter is microcrystalline cellulose. In some embodiments, the hydrophilic carrier of the subcoat is a combination of povidone and microcrystalline cellulose.

In some embodiments, at least about 80% of the naltrexone is released within about 1 hour after the delayed burst release occurs. In some embodiments, the water insoluble hydrophilic particulate matter forms channels in the outer coating upon contact with a liquid, whereby the channels absorb body liquid and cause at least one burst controlling agent to burst the coating, thereby providing delayed burst release of the naltrexone. In some embodiments, the cross-linked polysaccharide is selected from the group consisting of insoluble metal salts or cross-linked derivatives of alginate, pectin, xanthan gum, guar gum, tragacanth gum, locust bean gum, and carrageenan. In some embodiments, the modified cellulose is selected from the group consisting of cross-linked derivatives of hydroxypropylcellulose, hydroxypropyl methylcellulose, hydroxyethyl cellulose, methylcellulose, carboxymethylcellulose, and a metal salt of carboxymethylcellulose. In some embodiments, the water insoluble polymer is talc, microcrystalline cellulose or a combination thereof.

In some embodiments, core further comprises at least one disintegrant, wherein the disintegrant is selected from the group consisting of cross-linked polyvinylpyrrolidone, sodium starch glycolate, cross-linked sodium carboxymethylcellulose, pregelatinized starch, microcrystalline starch, water insoluble starch, calcium carboxymethylcellulose, low substituted carboxymethylcellulose, low substituted hydroxypropyl cellulose, magnesium aluminum silicate, and combinations thereof.

### EXAMPLES

The disclosure is further illustrated by the examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications and equivalents thereof which may suggest themselves to those skilled in the art without departing from the spirit of the present disclosure and/or scope of the appended claims.

### Example 1—Preparation for Naltrexone Hydrochloride Delayed Burst-Release Formulation

This example describes the preparation of naltrexone hydrochloride delayed burst-release formulation.

### Drug Layering—Core Preparation

A core was prepared with a composition according to Table 1.

TABLE 1
Core composition-Example 1

| Component | Percent w/w (%) | Quantity per batch (g) |
|---|---|---|
| Sugar Spheres | 97.0 | 1000.0 |
| Naltrexone HCl | 2.5 | 25.8 |
| Hypromellose | 0.25 | 2.6 |
| Talc | 0.25 | 2.6 |
| Total | 100.0 | 1031.0 |

The core was prepared by the manufacturing process:
Naltrexone HCl and hypromellose were dissolved in purified water.
Talc was added and mixed until uniformly dispersed.
Sugar spheres were charged into the fluid bed processor.
The drug dispersion was layered onto the sugar spheres to attain the target weight gain.
The cores were dried to remove retained water.

### Coating

Coating was performed on the coated naltrexone HCl cores using the membrane coating composition according to Table 2. 700 g of naltrexone cores from Example 1 were used for the coating process. Coating was performed in a fluid bed processor equipped with a Wurster column using the EUDRAGIT® L 100 dispersion composition given in Table 2. Coating was performed to a target weight gain of 20% w/w and samples were removed at different levels to evaluate dissolution to determine target weight gain.

TABLE 2
Coating composition-Example 1

| Component | Percent w/w (%) |
|---|---|
| EUDRAGIT ® L 100 | 62.5 |
| TEC | 6.3 |
| Talc | 31.2 |
| Total | 100.0 |

### Example 2—Preparation for Naltrexone Hydrochloride Delayed Burst-Release Formulation

This example describes the preparation of naltrexone hydrochloride delayed burst-release formulation.

### Drug Layering—Core Preparation

A core was prepared with the composition according to Table 3.

TABLE 3
Core composition-Example 2

| Component | Percent w/w (%) | Quantity per batch (g) |
|---|---|---|
| Microcrystalline cellulose | 97.0 | 1500.0 |
| spheres | | |
| Naltrexone HCl | 2.0 | 30.9 |
| Hypromellose | 0.25 | 3.9 |
| Croscarmellose sodium | 0.25 | 3.9 |
| Talc | 0.50 | 7.7 |
| Total | 100.0 | 1546.4 |

Manufacturing Process:
Naltrexone HCl, hypromellose and croscarmellose sodium were dissolved in ethanol-water co-solvent system.
Talc was added and mixed until uniformly dispersed.
MCC spheres were charged into the fluid bed processor.
The drug dispersion was layered onto the MCC spheres to attain the target weight gain.
The cores were dried to remove excess ethanol.

### Delayed Release Coating

Functional coating was performed on the coated naltrexone HCl cores using the membrane coating composition according to Table 4. 700 g of naltrexone cores from Example 2 were used for the coating process. Coating was performed in a fluid bed processor equipped with a Wurster column using the EUDRAGIT® L 100 dispersion composition given in Table 4 in a suitable solvent system. Coating was performed to a target weight gain of 25% w/w and samples were removed at different levels to evaluate dissolution to determine target weight gain.

TABLE 4
Coating composition-Example 2

| Component | Percent w/w (%) |
|---|---|
| EUDRAGIT ® L 100-55 | 64.5 |
| TEC | 3.2 |
| Talc | 32.3 |
| Total | 100.0 |

### Example 3—Preparation for Naltrexone Hydrochloride Delayed Burst-Release Formulation

This example describes the preparation of naltrexone hydrochloride delayed, burst-release formulation.

### Core Production

A core was prepared with the composition according to Table 5.

TABLE 5
Core composition-Example 3

| Component | Percent w/w (%) | Quantity per batch (g) |
|---|---|---|
| Naltrexone HCl | 5.0 | 40.0 |
| Crospovidone | 5.0 | 40.0 |
| Microcrystalline cellulose | 85.0 | 680.0 |
| (MCC) | | |
| Hydroxypropyl cellulose | 5.0 | 40.0 |
| Total | 100.0 | 800.0 |

Manufacturing Process:
Naltrexone HCl, crospovidone and MCC were screened through a mesh #40 sieve to de-agglomerate.
The material was charged into a high shear granulator and mixed.
Hydroxypropyl cellulose was dissolved in purified water to prepare the granulation aid.
The mixture was granulated using the granulation aid until granule formation was observed.
The granules were then passed through an extruder followed by spheronization to form naltrexone HCl cores.
Naltrexone HCl cores were further dried using a tray dryer or a fluid bed processor.

### Subcoat Coating

Seal coating was performed on the cores using the membrane coating composition of Table 6. 750 g of naltrexone HCl cores from Example 3 were coated with the seal coating dispersion from Table 6 using a fluid bed processor equipped with a Wurster column.

TABLE 6
Coating composition-Example 3

| Component | Percent w/w (%) |
|---|---|
| Hypromellose | 1.2 |
| Talc | 3.8 |
| Ethanol 190 proof | 95.0 |
| Total | 100.0 |

### Delayed Release Layer

Functional coating was performed on the seal coated naltrexone HCl cores using the membrane coating composition of Table 7. 700 g of seal coated naltrexone HCl cores from Example 3 were used for the coating process. Coating was performed in a fluid bed processor equipped with a Wurster column using the EUDRAGIT® L 100-55 dispersion composition from Table 7 in a suitable solvent system. Coating was performed to a target weight gain of 25% w/w and samples are removed at different levels to evaluate dissolution to determine target weight gain.

TABLE 7
Coating composition-Example 3

| Component | Percent w/w (%) |
|---|---|
| EUDRAGIT ® L 30 D55 | 64.5 |
| TEC | 3.2 |
| Talc | 32.3 |
| Total | 100.0 |

### Example 4—Preparation for Naltrexone Hydrochloride Delayed Burst-Release Formulation

This example describes the preparation of naltrexone hydrochloride delayed burst-release formulation using naltrexone HCl cores from Example 1.

900 g of Naltrexone HCl cores from Example 1 were used and an additional disintegrant layer was coated onto the cores using a fluid bed processor. The composition was as shown in Table 8:

TABLE 8
Disintegrant layer composition-Example 4

| Component | Percent w/w (%) | Quantity per batch (g) |
|---|---|---|
| Naltrexone HCl cores | 95.0 | 900.0 |
| (from Example 1) | | |
| Crospovidone | 4.0 | 37.9 |
| Kollidon ® 30 | 0.4 | 2.6 |
| Talc | 0.6 | 2.6 |
| Total | 100.0 | 943.1 |

Kollidon® 30 (BASF) is a medium molecular povidone (PVP).

Manufacturing Process:
Kollidon® 30 and crospovidone were dissolved in 200 proof ethanol.
Talc was added and mixed until uniformly dispersed.
Naltrexone HCl cores were charged into the fluid bed processor.
The coating dispersion was layered onto the cores.
The coated cores were dried to remove the excess ethanol.

### Delayed Release Coating

Functional coating was performed on the coated naltrexone HCl cores. 700 g of coated naltrexone cores from Example 1 were used for the coating process. Coating was performed in a fluid bed processor equipped with a Wurster column using the EUDRAGIT® L 100 dispersion composition given in Table 2 of Example 1. Coating was performed to a target weight gain of 20% w/w and samples were removed at different levels to evaluate dissolution to determine target weight gain.

### Example 5—Example Layers

Examples 5a-5c describe cores. Example 5d describes a subcoat. Examples 5e-g describe outer enteric delayed release coatings.

The cores contained the active pharmaceutical ingredient (API) naltrexone HCl or naloxone (a water soluble salt).

Example 5a: An example core with excipients had a core composition according to Table 9.

TABLE 9
Core composition-Example 5a

| Component | Percent w/w (%) | Quantity per batch (g) |
|---|---|---|
| Sugar Spheres | 97.0 | 1000.0 |
| Naltrexone HCl | 2.5 | 25.8 |
| hydroxypropyl | 0.25 | 2.6 |
| methylcellulose | | |
| Talc | 0.25 | 2.6 |
| Total | 100.0 | 1031.0 |

The Table 9 core composition was formed by (a) dissolving naltrexone HCl and hydroxypropyl methylcellulose (HPMC) in purified water; (b) adding talc and mixing until uniformly dispersed to form the drug dispersion; (c) charging sugar spheres into a fluid bed processor; and (d) layering the drug dispersion onto the charged sugar spheres to attain a target weight gain of cores. The cores were dried to remove retained water. In some embodiments, the core further comprises at least one disintegrant, wherein the disintegrant is selected from the group consisting of cross-linked polyvinylpyrrolidone, sodium starch glycolate, cross-linked sodium carboxymethylcellulose, pregelatinized starch, microcrystalline starch, water insoluble starch, calcium carboxymethylcellulose, low substituted carboxymethylcellulose, low substituted hydroxypropyl cellulose, magnesium aluminum silicate, and combinations thereof.

Example 5b: Another example core had a core composition according to Table 10:

TABLE 10
Core composition-Example 5b

| Component | Percent w/w (%) | Quantity per batch (g) |
|---|---|---|
| Naltrexone HCl | 5.0 | 40.0 |
| Cross linked polyvinyl N- | 5.0 | 40.0 |
| pyrrolidone (PVP) | | |
| Microcrystalline cellulose | 85.0 | 680.0 |
| (MCC) | | |
| Hydroxypropyl cellulose | 5.0 | 40.0 |
| Total | 100.0 | 800.0 |

Naltrexone HCl, PVP and MCC were screened through a mesh #40 sieve to de-agglomerate. The material was charged into a high shear granulator and mixed. Hydroxypropyl cellulose was dissolved in purified water to prepare the granulation aid. The mixture was granulated using the granulation aid until granule formation was observed. The granules were then passed through an extruder followed by spheronization to form naltrexone HCl cores. The naltrexone HCl cores were further dried using a tray dryer or a fluid bed processor.

Example 5c: Another example core had a core composition according to Table 11:

TABLE 11
Core composition-Example 5c

| | Percent | Quantity |
|---|---|---|
| Component | w/w (%) | per batch (g) |
| Microcrystalline cellulose spheres | 97.0 | 1500.0 |
| Naltrexone HCl | 2.0 | 30.9 |
| Hydroxypropyl methylcellulose | 0.25 | 3.9 |
| Cross-linked sodium | 0.25 | 3.9 |
| carboxymethylcellulose | | |
| Talc | 0.50 | 7.7 |
| Total | 100.0 | 1546.4 |

Naltrexone HCl, hydroxypropyl methylcellulose and cross-linked sodium carboxymethylcellulose were dissolved in an ethanol-water cosolvent system. Talc was added and mixed until uniformly dispersed. MCC spheres were charged into a fluid bed processor to form a drug dispersion. The drug dispersion was layered onto the MCC spheres to attain the target weight gain to form cores. The cores were dried to remove excess ethanol. Cross-linked sodium carboxymethylcellulose was used as a disintegrant.

Example 5d describes a subcoat with a subcoat composition according to Table 12:

TABLE 12
Coating composition-Example 5d

| Component | Percent w/w (%) |
|---|---|
| Hydroxypropyl methylcellulose | 1.2 |
| Talc | 3.8 |
| Ethanol 190 proof | 95.0 |
| Total | 100.0 |

750 g of Naltrexone HCl cores were coated with the seal coating dispersion from Table 12 using a fluid bed processor equipped with a Wurster column to form subcoated cores.

Example 5e describes an outer enteric delayed release coating.

Outer enteric delayed release coating was performed on the cores using the membrane coating composition of Table 13:

TABLE 13
Outer enteric delayed release coating
composition-Example 5e

| Component | Percent w/w (%) |
|---|---|
| EUDRAGIT ® L 100 | 62.5 |
| TEC | 6.3 |
| Talc | 31.2 |
| Total | 100.0 |

EUDRAGIT® L 100 (Evonik) is a powder for dissolution above pH 6.0. 700 g of naltrexone cores were used for the coating process. Coating was performed in a fluid bed processor equipped with a Wurster column using the EUDRAGIT® L 100 dispersion composition given in Table 13. Coating was performed to a target weight gain of 20% w/w and samples were removed at different levels to evaluate dissolution to determine target weight gain.

Example 5f describes an outer enteric delayed release coating.

Outer enteric delayed release coating was performed on the cores using the membrane coating composition of Table 14:

TABLE 14
Outer enteric delayed release coating
composition-Example 5f

| Component | Percent w/w (%) |
|---|---|
| EUDRAGIT ® L 100 | 62.5 |
| TEC | 6.3 |
| Talc | 31.2 |
| Total | 100.0 |

700 g of naltrexone cores were used for the coating process. Coating was performed in a fluid bed processor equipped with a Wurster column using the EUDRAGIT® L 100 dispersion composition given in Table 14. Coating was performed to a target weight gain of 20% w/w and samples were removed at different levels to evaluate dissolution to determine target weight gain.

Example 5g describes an outer enteric delayed release coating.

Outer enteric delayed release coating was performed on the cores that had a subcoating using the membrane coating composition of Table 15:

TABLE 15
Outer enteric delayed release coating composition
for subcoated cores-Example 5g

| Component | Percent w/w (%) |
|---|---|
| EUDRAGIT ® L 30 D55 | 64.5 |
| TEC | 3.2 |
| Talc | 32.3 |
| Total | 100.0 |

700 g of subcoated naltrexone HCl cores were used for the outer enteric delayed release coating process. Coating was performed in a fluid bed processor equipped with a Wurster column using the EUDRAGIT® L 100-55 dispersion composition from Table 15 in deionized water. Coating was performed to a target weight gain of 25% w/w and samples were removed at different levels to evaluate dissolution to determine target weight gain.

### Example 6—Preparation for Naltrexone Hydrochloride Delayed Burst-Release Capsules

This example describes another example preparation of naltrexone hydrochloride delayed burst-release capsules.

### Disintegrant Layer

900 g of naltrexone HCl cores were used and an additional disintegrant layer was coated onto the cores using a fluid bed processor. The compositions was as shown in Table 16.

TABLE 16
Core composition-Example 6

| | Percent | Quantity |
|---|---|---|
| Component | w/w (%) | per batch (g) |
| Naltrexone HCl cores | 95.0 | 900.0 |
| Cross linked polyvinyl | 4.0 | 37.9 |
| N-pyrrolidone, or PVP | | |
| Kollidon ® 30 | 0.4 | 2.6 |
| Talc | 0.6 | 2.6 |
| Total | 100.0 | 943.1 |

Kollidon® 30 (BASF) is a medium molecular povidone (PVP).

### Manufacturing Process

Kollidon® 30 and cross linked polyvinyl N-pyrrolidone were dissolved in 200 proof ethanol. Talc was added and mixed until uniformly dispersed to form a coating dispersion. Naltrexone HCl cores were charged into a fluid bed processor. The coating dispersion was layered onto the charged cores to form coated cores. The coated cores were dried to remove the excess ethanol.

Outer enteric delayed release coating was applied to the coated naltrexone HCl cores. 700 g of coated naltrexone cores was used for the coating process. Coating was performed in a fluid bed processor equipped with a Wurster column using an EUDRAGIT® L 100 dispersion. Coating was performed to a target weight gain of 20% w/w and samples were removed at different levels to evaluate dissolution to determine target weight gain.

In some examples, naltrexone HCl (API) was incorporated into cores, with or without disintegrant, followed by outer enteric delayed release coating with a pH-dependent release controlling polymer. The delayed burst effect was achieved by the pH-dependent release-controlling polymer that protects the API from being released into the acidic environment of the stomach. Once the pellets were moved to a higher pH (around pH 5.0), the film coating started to erode/dissolve which allowed water to penetrate into the core (with active API) to explode with or without the aid of a disintegrant in a burst release of API (naltrexone).

### Example 7—4 mg LDN (Low Dose Naltrexone) Formulation

This example describes preparation of a 4 mg LDN (low dose naltrexone) formulation which had a core according to Table 17 and an outer enteric delayed release coating according to Table 18. This formulation may be provided in a tablet form.

TABLE 17
Core composition-Example 7

| Excipient | Mg/tablet | % |
|---|---|---|
| Naltrexone HCl | 1.0 to 4.0 mg | 2.00 |
| Lactose monohydrate 100M | 20.00 | 6.67 |
| Crosslinked sodium | 1.30 | 0.43 |
| carboxymethylcellulose | | |
| Microcrystalline cellulose pH 101 | 19.55 | 6.52 |
| Ascorbic acid | 3.00 | 1.00 |
| Providone K-30 | 3.10 | 1.03 |
| (polyvinylpyrrolidone) | | |
| Citric acid anhydrous | 1.50 | 0.50 |
| Butyl hydroxyanidole | 0.05 | 0.02 |
| Dry blend Mixture | | |
| Microcrystalline cellulose PH 102 | 225 | 76.00 |
| Crosslinked sodium | 6.00 | 2.00 |
| carboxymethylcellulose | | |
| Colloidal silicon dioxide | 6.00 | 2.00 |
| Magnesium stearate | 1.90 | 0.63 |
| Total | 300.00 | 100 |

TABLE 18
Outer enteric delayed release coating-Example 7

| Excipient | Mg/tablet |
|---|---|
| Microcrystalline cellulose | 21.9 |
| (Avicel ® PH 102) | |
| Ethylcellulose (Ethocel 20) | 14.6 |
| Cetyl alcohol | 1.5 |
| Total | 38.0 |

### Example 8—Low Dose Naltrexone Granules

This example describes preparation of low dose naltrexone granules which had a composition according to Table 19.

TABLE 19
Composition of low dose naltrexone granules-Example 8

| Excipient | Mg/tab | % |
|---|---|---|
| Wet granulation mixture | | |
| Naltrexone HCl | 1·4.00 | 2 |
| Lactose monohydrate 100M | 60.00 | 18.00 |
| Crosslinked sodium carboxymethylcellulose | 3.20 | 1.00 |
| Microcrystalline cellulose (Avicel ® PH 101) | 48.90 | 15.28 |
| Ascorbic acid | 7.50 | 2.34 |
| Polyvinyl pyrrolidone (Povidone K-30) | 7.70 | 2.41 |
| Citric acid anhydrous | 3.75 | 1.17 |
| Butyl hydroxyanisole | 0.12 | 0.04 |
| Dry Bled Mixture | | |
| Microcrystalline cellulose (Avicel ® PH 102) | 144.00 | 45 |
| Crosslinked sodium carboxymethylcellulose | 6.40 | 2.00 |
| Colloidal silicon dioxide | 6.40 | 2.00 |
| Magnesium stearate | 2.00 | 0.64 |
| Total | 300.00 | 100 |
| Excipient | | |
| Microcrystalline cellulose (Avicel ® PH 102) | 23.1 | |
| Ethylcellulose (Ethocel 20) | 15.4 | |
| Cetyl alcohol | 1.5 | |
| Total | 40.0 | |

### Example 9—Layered LDN Cores

This example describes preparation of layered cores containing LDN.

A dispersion of naltrexone was prepared as follows: To 5.725 kg of deionized water was added 0.113 kg of hydroxypropyl methylcellulose and 200 g of naltrexone, followed by moderate mixing, using a stirring paddle for about 30 min to form a drug dispersion. The drug dispersion was sprayed onto sugar seeds (20/35 mesh) is a 9″ Wurster Column of GPCG-fluid bed processor. After the entire dispersion was applied, the cores were dried in the column for 5 min. The drug-loaded cores were discharged from the column and passed through a 20 mesh screen. A protective coat (such as OPADRY beige) was applied onto the IR beads to provide color and physical protection.

### Example 10—Enteric Coated LDN Cores

This example describes preparations of a formulation of enteric coated LDN cores.

An EUDRAGIT® L 30 D-55 coating dispersion was prepared by adding 0.127 kg of trimethyl citrate into 3.538 kg of EUDRAGIT® L 30 D-55 (solid content: 1.6061 kg) and stirring for at least 30 min. Talc (0.315 kg) was dispersed into 2.939 kg of deionized water. The plasticized EUDRAGIT® L 30 D-55 was combined with the talc dispersion and screened through a 60 mesh screen. The resulting combined dispersion was sprayed onto drug-loaded cores (3.5 kg) prepared according to Example 3 in a 9″ Wurster Column of a CPCG-15 fluid bed processor. A protective coat was applied (OPADRY beige).

### Example 11—Delayed Burst Release Tablet

This example describes preparation of a delayed burst release tablet of LDN.

LDN (200 g) was blended with 3.15 kg of monocrystalline cellulose in a V-shaped blender for 15 min and the powder blend was then lubricated with magnesium stearate (0.0375) for an additional 5 min. Doxcycline monohydrate (0.2 kg) was granulated with EUDRAGIT® L 100 powder (1.280 kg) and monocrystalline cellulose powder (0.5 kg) using isopropyl alcohol as a granulating fluid. The wet granulation was dried in a fluid bed dryer and then the dried granules were blended with magnesium stearate (0.020 kg) in a V-shaped blender for 5 min. Doxycycline powder blend and the granulation were put on a belayed tablet press to compress into a belayed tablet with target weights of 180 mg and 85 mg for the powder blend and granulation layers, respectively.

### Example 12—Formulation Screening of Naltrexone HCl (LDN) DR Capsules, 2 mg

A multi-particulate system containing cores coated with an enteric delayed-release polymer was chosen as a formulation approach for LDN DR Capsules, 2 mg. The multi-particulate approach provided 1) better consistency and less variation in drug release by minimizing food effect in-vivo; 2) ease of administration (swallow and sprinkle); and 3) the dose could be easily titrated for different dose strengths.

### Excipient Selection and Compatibility Studies

Based on the formulation approach and our experience, various excipients were chosen on account of their specific functions. To assess the compatibility of each excipient with the drug, a drug-excipient compatibility study was designed and performed.

### Formulation Development

Initial formulation experiments were designed with a core layer, a disintegrant layer, and a delayed release coating. Example formulae and their processing were as described in Table 20:

TABLE 20
Naltrexone HCl DR capsules, 2 mg formulation-Example 12

| | | | % w/w | |
|---|---|---|---|---|
| Stage | Material | Function | Formulation 1 | Formulation 2 |
| Drug | Sugar Spheres 30/35 mesh | Inert Core | 96.95 | |
| layering | Naltrexone HC1 | API | 2.54 | |
| | Hydroxypropyl | Binder | 0.25 | |
| | methylcellulose | | | |
| | (HPMC E5) | | | |
| | Talc | Anti-tacking | 0.25 | |
| | | agent | | |
| | Purified water | Solvent | N/A | |
| | Total | N/A | 100.0 | |
| Disintegrant | Naltrexone HCl Cores | Substrate | N/A | 95.24 |
| Layering | Croscarmellose Sodium | Super- | N/A | 3.97 |
| | | disintegrant | | |
| | Hydroxypropyl | Binder | N/A | 0.40 |
| | methylcellulose | | | |
| | (HPMC E5) | | | |
| | Talc | Anti-tacking | N/A | 0.40 |
| | | agent | | |
| | Ethanol 190 Proof | Solvent | N/A | N/A |
| | Total | N/A | N/A | 100.0 |
| | Naltrexone HCl Cores | Substrate | 83.33 | N/A |
| Delayed | Naltrexone HCl Cores with | Substrate | N/A | 76.92 |
| Release | disintegrant coat | | | |
| Coating | EUDRAGIT ® L 100-55 | Delayed | 11.11 | 3.59 |
| | | Release | | |
| | | polymer | | |
| | EUDRAGIT ® L 30 D-55 | Delayed | N/A | 11.79 |
| | | Release | | |
| | | polymer | | |
| | Talc | Anti-tacking | 5.56 | 7.69 |
| | | agent | | |
| | Ethanol 190 Proof | Solvent | N/A | N/A |
| | Acetone | Solvent | N/A | N/A |
| | IPA | Solvent | N/A | N/A |
| | Purified Water | Diluent | N/A | N/A |
| | Total | N/A | 100.0 | 100.0 |

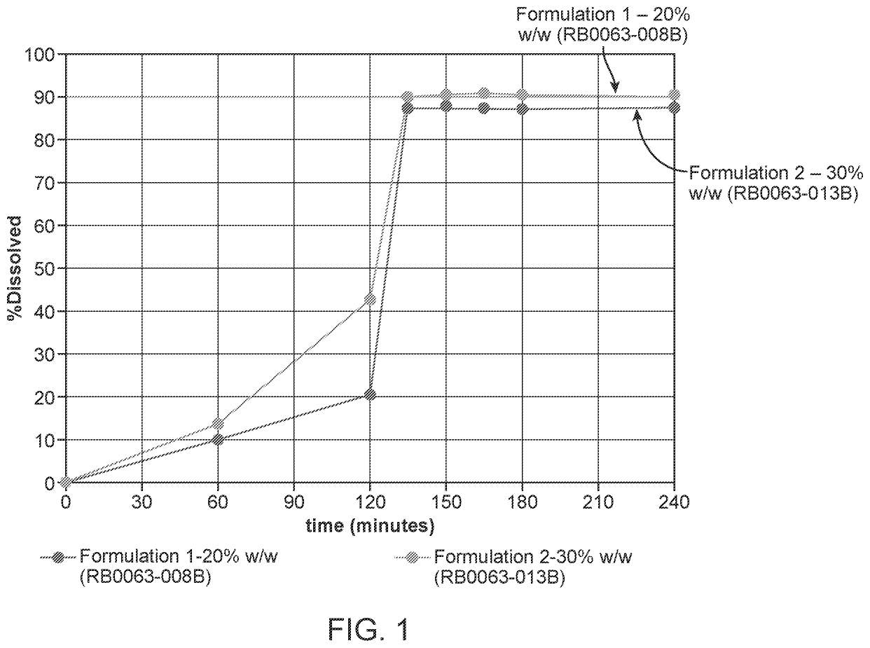

All stages of the naltrexone HCl DR pellets were manufactured using a Freund Vector VFC LAB-1 fluid bed processor equipped with Wurster coating assembly.
Cores were produced by layering a drug dispersion from Table 20 on to mesh #30/35 sugar spheres for both Formulations 1 and 2.
An additional disintegrant layer was coated on to the cores to produce the LDN cores with a disintegrant layer to facilitate a burst release of the drug.
The delayed release coating for Formulation 1 was prepared using EUDRAGIT® L 100-55 dispersion in ethanol and coated up to 20% w/w. High static and agglomeration were observed during the coating process.
The delayed release coating for Formulation 2 was prepared using an EUDRAGIT® L 100-55 dispersion in acetone, IPA, and a water co-solvent system and coated up to 7% w/w. The process was stopped due to excessive static and agglomeration. Coating was further continued using EUDRAGIT® L 30 D-55 aqueous dispersion to avoid static build-up. Coating was successfully completed up to 30% w/w.
Samples from Formulation 1 (Lot #RB0063-008B) of 20% coat and 2 (Lot #RB0063-013B) of 30% coat were tested for dissolution, and results are given in FIG. 1.

Dissolution data in the acid stage showed premature release of drug, suggesting the coating weight gain was not sufficient for both Formulations 1 and 2. It was planned to employ an increase in coating level in future developments. In addition, the premature release of drug in 30% coating was more than for the 20% coating, which suggested the use of a disintegrant as well as addition of a seal coat may be necessary.

The oral delayed burst formulations for the next trials were modified to incorporate superdisintegrant within the drug layer as opposed to coating as a separate layer. An additional seal coat was applied on to the cores to provide a smooth surface and facilitate the application of delayed release coating on to the cores, and to ensure drug stability upon storage. EUDRAGIT® L 30 D-55 was chosen as the delayed release polymer due to its pH dependent characteristics at a pH of 5.5. Should slight delayed onset be required, other DR polymers with higher pH dependent properties can be applied.

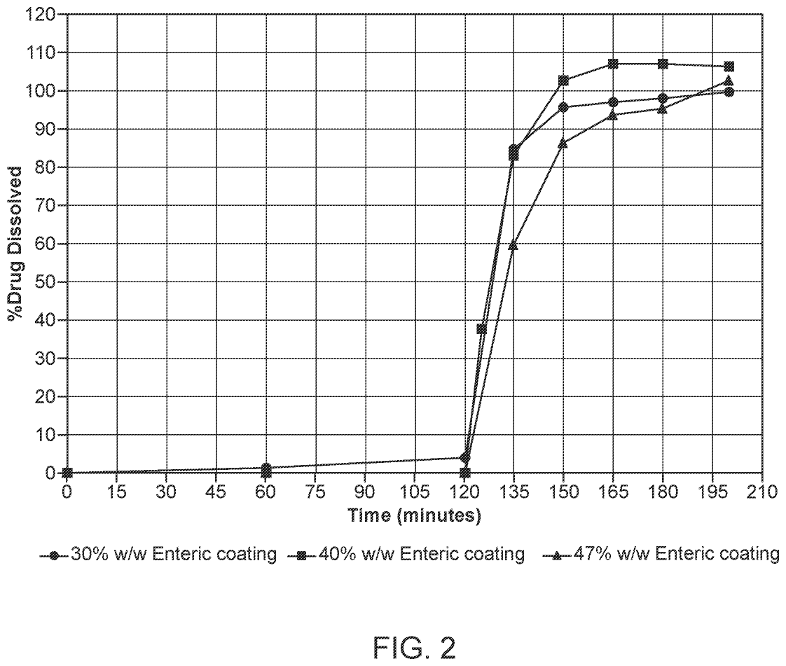

DR coating was performed up to a weight gain of 47% w/w and samples were collected at 30% w/w and 40% w/w and tested for dissolution in 2-stage dissolution media, 0-120 mins in 0.1N HCl and 120-200 mins in pH 5.5 buffer. The results are shown in FIG. 2.

Dissolution data showed slight premature release of drug in the acid stage for the 30% w/w coating weight gain sample. For the samples with 40% w/w and 47% w/w of DR coating level, no release was observed in the acid stage and more than 85% of the drug was released in the buffer stage in 30 minutes. To assure the integrity of the coating and no release in acidic environment, the 47% coating level was selected as the prototype formula for proof-of-concept study and may be fine-tuned once at the scale-up stage.

### Analytical Method

The 2-stage dissolution method used in the development is provided in Example 21

For the naltrexone assay, the USP monograph for naltrexone HCl tablets was used. Refer to USP43-NF38-3069.

### Examples 13-16—Naltrexone HCl Delayed-Release Capsules—Initial Formulation Trials

Initial formulation of naltrexone hydrochloride DR pellets was developed by a three step process including 1) Drug layering: a dispersion of hydroxypropyl methylcellulose (HPMC E5), naltrexone HCl and talc in purified water was layered on to sugar spheres, followed by 2) Disintegrant layering: a dispersion of hydroxypropyl methylcellulose (HPMC E5), croscarmellose sodium and talc in 190 proof ethanol was layered on to cores, and finally by 3) Outer enteric polymeric coating: a dispersion of EUDRAGIT® L 100-55 and talc in acetone, IPA and purified water was layered on to the disintegrant coated drug cores. Process difficulties were encountered during the organic-solvent based enteric coating trials, and an aqueous enteric coating dispersion EUDRAGIT® L 30 D-55 was evaluated. The process difficulties such as static and agglomeration were resolved by using the aqueous coating.

In a subsequent trial, the disintegrant was incorporated into the drug layering dispersion. Due to this change, the solvent for drug layering was changed from purified water to a cosolvent system consisting of methanol and purified water in a 95:5 ratio. The rationale for this change was to choose a solvent that can solubilize naltrexone HCl, solubilize the hydroxypropyl methylcellulose (HPMC E5) which was sparingly soluble in 100% methanol, and not cause swelling of croscarmellose sodium. Croscarmellose, although insoluble in water, rapidly swells when in contact with water. An additional seal coating layer was then applied on to the cores by coating a dispersion on hydroxypropyl methylcellulose (HPMC E5) and talc in 190 proof ethanol. An outer enteric coating was then applied on to the seal coated cores using aqueous coating dispersion containing EUDRAGIT® L 30 D-55, talc and TEC. The optimum coating level based on evaluations was 47% w/w.

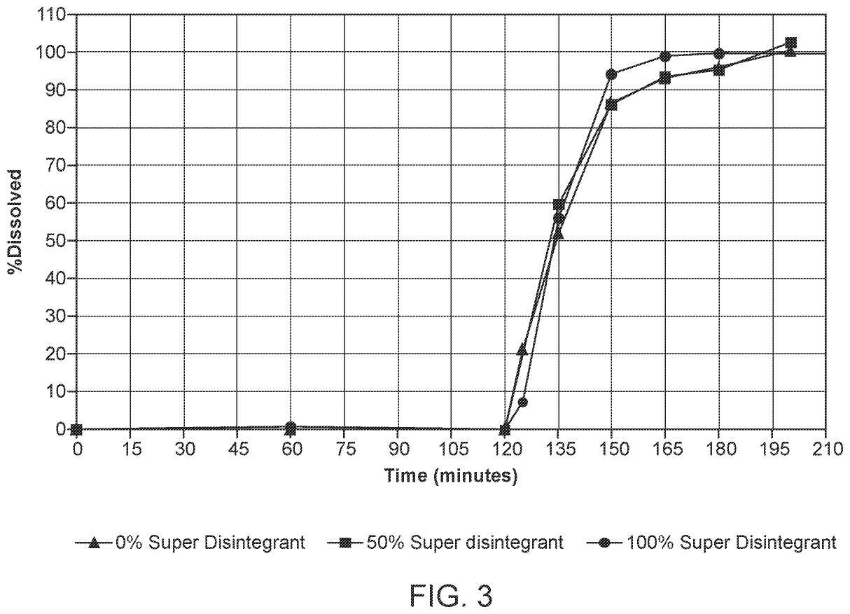

Different levels of croscarmellose sodium, 0% w/w, 50% w/w and 100% w/w with respect to the API in formulation, were evaluated to find the optimum concentration required to provide a desired burst effect. Results are shown in Example 17 and FIG. 3.

The composition, process description and process parameters for the initial product development trials for naltrexone hydrochloride delayed-release formulations for capsules are described in Examples 13 through 16.

### Example 13—Naltrexone Hydrochloride Delayed-Release Formulation

This example describes preparation of naltrexone hydrochloride delayed-release formulation.

### Drug Layering—Core Composition

The core had a composition according to Table 21.

TABLE 21
Drug layering Formulation Composition-Example 13

| | Percent | Quantity |
|---|---|---|
| Component | w/w (%) | per batch (g) |
| Sugar spheres 30/35 mesh | 96.9 | 1600.0 |
| Naltrexone HCl | 2.54 | 42.0 |
| Hydroxypropyl | 0.25 | 4.2 |
| methylcellulose 5 mpas | | |
| (HPMC E5) | | |
| Talc | 0.25 | 4.2 |
| Purified water | N/A | 669.6 |
| Total | 100.0 | 1650.4 |

Process:
HPMC was dissolved in purified water to obtain a clear solution using an over-head mixer.
Naltrexone HCl was then added and mixing was continued until completely dissolved.
Talc was added to the solution and mixed for 20 minutes.
1600 g of 30/35 mesh sugar spheres were charged into a VFC-Lab 1 FLO-COATER® equipped with a Wurster coating system.
The drug dispersion was layered onto the sugar sphere cores to attain the target weight gain of approximately 3.2% w/w.
Process parameters for the drug layering process were as shown in Table 22.
The cores were dried in the fluid bed processor for 10 minutes and allowed to cool down before discharge.

TABLE 22
Drug layering process parameters-Example 13

| Operational Parameter | Target setpoint/range |
|---|---|
| Spray rate | 5-10 g/min |
| Nozzle air | 24-28 psi |
| Cylinder air | 18-22 psi |
| Inlet air volume | 40-50 cfm |
| Inlet air temperature | 50-54° C. |
| Product temperature | 40-45° C. |

### Disintegrant Layer

800.0 g of naltrexone HCl cores produced by the drug layering process in Example 13 were coated with the disintegrant dispersion from Table 23 using a VFC-Lab 1 FLO-COATER® equipped with a Wurster coating system. The process parameters are given in Table 24.

TABLE 23
Disintegrant Layering Formulation Composition-Example 13

| | Percent | Quantity per |
|---|---|---|
| Component | w/w (%) | batch (g) |
| Naltrexone hydrochloride | 95.2 | 800.0 |
| cores | | |
| Croscarmellose sodium | 4.0 | 33.33 |
| Hydroxypropyl | 0.4 | 3.33 |
| methylcellulose 5 mpas | | |
| (HPMC E5) | | |
| Talc | 0.4 | 3.33 |
| Ethanol 190 Proof | N/A | 360.00 |
| Purified water | N/A | 20.0 |
| Total | 100.0 | 840.0 |

Coating Process:
HPMC was added to Ethanol 190 proof and mixed using an over-head mixer.
Purified water was added and mixing was continued until a clear solution was formed.
Croscarmellose sodium was added and mixing was continued for no longer than 10 minutes.
Talc was then added and mixing was continued for no longer than 10 minutes.
800 g of naltrexone hydrochloride cores from Example 13 were charged into VFC-Lab 1 FLO-COATER® equipped with a Wurster coating system.
410 g of drug dispersion was layered on to the cores, equivalent to 5% w/w weight gain.
Process parameters for the drug layering process are given in Table 24.
The disintegrant layered cores were dried in the fluid bed processor for 5 minutes and allowed to cool down before discharge.

TABLE 24
Disintegrant layering process parameters-Example 13

| Operational Parameter | Target setpoint/ range |
|---|---|
| Spray rate | 5-8 g/min |
| Nozzle air | 23-26 psi |
| Cylinder air | 16-18 psi |
| Inlet air volume | 45-50 cfm |
| Inlet air temperature | 35-40° C. |
| Product temperature | 28-34° C. |

### Delayed Release Layer

700 g of the naltrexone HCl disintegrant layered cores were coated with the enteric polymer coating dispersion from Table 25 using a VFC-Lab 1 FLO-COATER® equipped with a Wurster coating system. The process parameters are given in Table 26.

TABLE 25
Enteric Polymer Coating Formulation
Composition-Example 13

| | Percent | Quantity |
|---|---|---|
| Component | w/w (%) | per batch (g) |
| Naltrexone HCl disintegrant | 78.8 | 700.0 |
| layered cores | | |
| Methacrylic Acid and Ethyl | 14.1 | 125.79 |
| Acrylate Copolymer | | |
| (EUDRAGIT ® L 100-55) | | |
| Talc | 7.1 | 62.89 |
| Acetone | N/A | 690.11 |
| Isopropyl Alcohol | N/A | 1034.87 |
| Purified Water | N/A | 86.34 |
| Total | 100.0 | 888.7 |

Enteric Polymer Coating Process:
Acetone, IPA and purified water were mixed in a 4 L glass beaker using an overhead mixer.
EUDRAGIT® L 100-55 was then added to the solvent system and mixed until completely dissolved.
Talc was added to the solution and mixed for no longer than 10 minutes.
700 g of naltrexone HCl disintegrant layered cores were charged into a VFC Lab-1 FLO-COATER.
The enteric polymer coating dispersion was layered onto the cores to attain a final weight gain of approximately 7% w/w.
The pellets were dried in the fluid bed processor for 10 minutes with no heat before discharge.

TABLE 26
Enteric coating process parameters - Example 13

| Operational Parameter | Target setpoint/range |
|---|---|
| Spray rate | 3-8 g/min |
| Nozzle air | 18-27 psi |
| Cylinder air | 13-22 psi |
| Inlet air volume | 45-55 cfm |
| Inlet air temperature | 24-27° C. |
| Product temperature | 21-25° C. |

### Outer Enteric Polymer Coating

640 g of the screened enteric coated naltrexone HCl cores (7% w/w enteric coating) were further coated with an aqueous dispersion of enteric polymer (EUDRAGIT® L 30 D-55) from Table 27 using a VFC-Lab 1 FLO-COATER® equipped with Wurster coating system. The process parameters are given in Table 28.

TABLE 27
Enteric Polymer Coating Formulation Composition - Example 13

| | | Quantity per |
|---|---|---|
| Component | Percent w/w (%) | batch (g) |
| Screened enteric coated | 66.5 | 640.0 |
| Naltrexone HCl cores (7% w/w | | |
| enteric coated) | | |
| Methacrylic Acid and Ethyl | 29.1 | 280.0 |
| Acrylate Copolymer Dispersion | | |
| (EUDRAGIT ® L 30 D-55, 30% | | |
| dispersion) | | |
| Talc | 4.4 | 42.0 |
| Purified Water | N/A | 341.6 |
| Total | 100.0 | 962.0 |

Enteric Polymer Coating Process:
A dispersion of talc in purified water was prepared using an overhead mixer.
Talc dispersion was added to EUDRAGIT® L 30 D-55 slowly while mixing using an overhead mixer.
Mixing was performed for no longer than 15 minutes.
640 g of screened enteric coated naltrexone HCl cores (7% w/w enteric coated) were charged into a VFC Lab-1 FLO-COATER.
The enteric polymer coating dispersion was layered onto the cores to attain a final weight gain of approximately 30% w/w.
The pellets were dried in the fluid bed processor for 5 minutes with no heat before discharge.

TABLE 28
Enteric coating process parameters - Example 13

| Operational Parameter | Target setpoint/range |
|---|---|
| Spray rate | 4-8 g/min |
| Nozzle air | 23-25 psi |
| Cylinder air | 18-22 psi |
| Inlet air volume | 45-52 cfm |
| Inlet air temperature | 28-38° C. |
| Product temperature | 25-30° C. |

### Example 14—Naltrexone Hydrochloride Delayed-Release Formulation

This example describes preparation of naltrexone hydrochloride delayed-release formulation.

### Drug Layering—Core Composition

The core had a composition according to Table 29.

TABLE 29
Drug layering formulation composition - Example 14

| Component | Percent w/w (%) | Quantity per batch (g) |
|---|---|---|
| Sugar spheres 25/30 mesh | 95.7 | 3000.0 |
| Naltrexone HCl | 2.55 | 79.9 |
| Hydroxypropyl | 0.25 | 8.0 |
| methylcellulose 5 mpas | | |
| (HPMC E5) | | |
| Talc | 0.25 | 8.0 |
| Croscarmellose sodium | 1.27 | 39.9 |
| Methanol | N/A | 1714.0 |
| Purified water | N/A | 90.2 |
| Total | 100.0 | 3135.8 |

Process:
HPMC was dissolved in a mixture of equal quantities of purified water and methanol to obtain a clear solution using an over-head mixer.
The remaining quantity of methanol was added to the HPMC solution and mixing was continued.
Naltrexone HCl was then added and mixing was continued until completely dissolved.
Talc was added to the solution and mixed for 15 minutes.
Croscarmellose sodium was added to the dispersion and mixed for no longer than 10 minutes.
3000 g of 25/30 mesh sugar spheres were charged into a VFC-Lab 3 FLO-COATER® equipped with a Wurster coating system.
The drug dispersion was layered onto the sugar sphere cores to attain the target weight gain of approximately 4.53% w/w.
Process parameters for the drug layering process were as shown in Table 30.

TABLE 30
Drug layering process parameters - Example 14

| Operational Parameter | Target setpoint/range |
|---|---|
| Spray rate | 4-17 g/min |
| Nozzle air | 27-29 psi |
| Cylinder air | 21-23 psi |
| Inlet air volume | 45-55 cfm |
| Inlet air temperature | 33-38° C. |
| Product temperature | 26-29° C. |

### Seal Coating

3000 g of the naltrexone HCl cores were further coated with a polymeric dispersion according to Table 31 using a VFC-Lab 3 FLO-COATER® equipped with a Wurster coating system. The process parameters were as shown in Table 32.

TABLE 31
Seal Coating Formulation Composition - Example 14

| Component | Percent w/w (%) | Quantity per batch (g) |
|---|---|---|
| Naltrexone HCl cores | 96.6 | 3000.0 |
| Hydroxypropyl methyl | 2.5 | 78.75 |
| cellulose 5 | | |
| mpas (HPMC E5) | | |
| Talc | 0.8 | 26.25 |
| Ethanol 190 Proof | N/A | 1995.00 |
| Total | 100.0 | 3105.0 |

Seal Coating Process:
HPMC was dissolved in ethanol 190 proof using an overhead mixer until a clear solution was formed.
Talc was then added to the solution and mixed for no longer than 15 minutes.
3000 g of the naltrexone HCl core was charged into a VFC Lab-3 FLO-COATER.
The seal coating dispersion was layered onto the cores to attain a final weight gain of approximately 3.5% w/w.

TABLE 32
Seal coating process parameters - Example 14

| Operational Parameter | Target setpoint/range |
|---|---|
| Spray rate | 7-21 g/min |
| Nozzle air | 28-30 psi |
| Cylinder air | 22-25 psi |
| Inlet air volume | 48-54 cfm |
| Inlet air temperature | 37-41° C. |
| Product temperature | 28-30° C. |

### Delayed Release Layer

700 g of the naltrexone HCl seal coated cores were coated with an aqueous dispersion of enteric polymer (EUDRAGIT® L 30 D-55) from Table 33 using a VFC-Lab 1 FLO-COATER® equipped with a Wurster coating system. The process parameters were as shown in Table 34.

TABLE 33
Enteric polymer coating formulation composition - Example 14

| | | Quantity |
|---|---|---|
| Component | Percent w/w (%) | per batch (g) |
| Naltrexone HCl seal coated cores | 66.7 | 700.0 |
| Methacrylic Acid and Ethyl | 20.8 | 729.2 |
| Acrylate Copolymer Dispersion | | |
| (EUDRAGIT ® L 30 D-55, 30% | | |
| dispersion) | | |
| Talc | 10.4 | 109.4 |
| Triethyl Citrate | 2.1 | 21.9 |
| Purified Water | N/A | 889.6 |
| Total | 100.0 | 1050.1 |

Enteric Polymer Coating Process:
TEC was added to purified water and mixed using an overhead mixer until completely miscible.
Talc was then added to the dispersion and continued mixing for no longer than 15 minutes.
Talc dispersion was added to EUDRAGIT® L 30 D-55 slowly while mixing using an overhead mixer.
Mixing was performed for no longer than 15 minutes.
700 g of the naltrexone HCl seal coated cores were charged into a VFC Lab-1 FLO-COATER.
The enteric polymer coating dispersion was layered onto the cores to attain a final weight gain of approximately 47% w/w.
The pellets were dried in the fluid bed processor for 5 minutes with no heat before discharge and cured at 40° C. for 2 hours in a tray dryer.

TABLE 34
Enteric coating process parameters - Example 14

| Operational Parameter | Target setpoint/range |
|---|---|
| Spray rate | 6-10 g/min |
| Nozzle air | 24-26 psi |
| Cylinder air | 21-23 psi |
| Inlet air volume | 47-52 cfm |
| Inlet air temperature | 38-41° C. |
| Product temperature | 28-31° C. |

### Example 15—Naltrexone Hydrochloride Delayed-Release Pellets

This example describes preparation of naltrexone hydrochloride delayed-release pellets.

### Drug Layering—Core Composition

The core had a composition according to Table 35.

TABLE 35
Drug layering formulation composition - Example 15

| Component | Percent w/w (%) | Quantity per batch (g) |
|---|---|---|
| Sugar Spheres 25/30 mesh | 96.9 | 3000.0 |
| Naltrexone HCl | 2.55 | 79.0 |
| Hydroxypropyl | 0.25 | 7.9 |
| methylcellulose 5 mpas | | |
| (HPMC E5) | | |
| Talc | 0.25 | 7.9 |
| Purified water | N/A | 1260.2 |
| Total | 100.00 | 3094.8 |

Process:
HPMC was dissolved in purified water to obtain a clear solution using an over-head mixer.
Naltrexone HCl was then added and mixing was continued until completely dissolved.
Talc was added to the solution and mixed for 15 minutes.
3000 g of 25/30 mesh sugar spheres were charged into VFC-Lab 3 FLO-COATER® equipped with a Wurster coating system.
The drug dispersion was layered onto the sugar sphere cores to attain the target weight gain of approximately 3.16% w/w.
Process parameters for the drug layering process were as shown in Table 36.

TABLE 36
Drug layering process parameters - Example 15

| Operational Parameter | Target setpoint/range |
|---|---|
| Spray rate | 6-10 g/min |
| Nozzle air | 25-27 psi |
| Cylinder air | 20-22 psi |
| Inlet air volume | 50-52 cfm |
| Inlet air temperature | 56-59° C. |
| Product temperature | 38-41° C. |

### Seal Coating

1500 g of the naltrexone HCl cores was further coated with a polymeric dispersion from Table 37 using a VFC-Lab 3 FLO-COATER® equipped with a Wurster coating system. The process parameters were as shown in Table 38.

TABLE 37
Seal coating formulation composition - Example 15

| | | Quantity per |
|---|---|---|
| Component | Percent w/w (%) | batch (g) |
| Naltrexone HCl cores | 96.6 | 1500.0 |
| Hydroxypropyl methylcellulose 5 | 2.5 | 39.4 |
| mpas (HPMC E5) | | |
| Talc | 0.8 | 13.1 |
| Ethanol 190 Proof | N/A | 997.5 |
| Total | 100.0 | 1552.5 |

Seal Coating Process:
HPMC was dissolved in ethanol 190 proof using an overhead mixer until a clear solution was formed.
Talc was then added to the solution and mixed for no longer than 15 minutes.
1500 g of the naltrexone HCl cores were charged into a VFC Lab-3 FLO-COATER.
The seal coating dispersion was layered onto the cores to attain a final weight gain of approximately 3.5% w/w.

TABLE 38
Seal coating process parameters - Example 15

| Operational Parameter | Target setpoint/range |
|---|---|
| Spray rate | 7-15 g/min |
| Nozzle air | 27-29 psi |
| Cylinder air | 21-23 psi |
| Inlet air volume | 47-49 cfm |
| Inlet air temperature | 40-42° C. |
| Product temperature | 28-30° C. |

### Delayed Release Layer

700 g of the naltrexone HCl seal coated cores were coated with an aqueous dispersion of enteric polymer (EUDRAGIT® L 30 D-55) using a VFC-Lab 1 FLO-COATER® equipped with a Wurster coating system. The composition and process of enteric coating was the same as for Example 14. The seal coated cores were coated to a target weight gain of 47% w/w and cured at 40° C. for 2 hours in a tray dryer.

### Example 16—Naltrexone Hydrochloride Delayed-Release Pellets

This example describes preparation of naltrexone hydrochloride delayed-release pellets.

### Core Composition

The core had a composition according to Table 39.

TABLE 39
Drug layering Formulation Composition - Example 16

| | | Quantity |
|---|---|---|
| Component | Percent w/w (%) | per batch (g) |
| Sugar spheres 25/30 mesh | 94.4 | 2000.0 |
| Naltrexone HCl | 2.55 | 54.0 |
| Hydroxypropyl methylcellulose | 0.25 | 5.4 |
| 5 mpas (HPMC E5) | | |
| Talc | 0.25 | 5.4 |
| Croscarmellose sodium | 2.55 | 54.0 |
| Methanol | N/A | 1141.1 |
| Purified water | N/A | 60.1 |
| Total | 100.00 | 2118.8 |

Process:
HPMC was dissolved in a mixture of equal quantities of purified water and methanol to obtain a clear solution using an over-head mixer.
The remaining quantity of methanol was added to the HPMC solution and mixing was continued.
Naltrexone HCl was then added and mixing was continued until completely dissolved.
Talc was added to the solution and mixed for 15 minutes.
Croscarmellose sodium was added to the dispersion and mixed for no longer than 10 minutes.
2000 g of 25/30 mesh sugar spheres were charged into a VFC-Lab 3 FLO-COATER® equipped with a Wurster coating system.
The drug dispersion was layered onto the sugar sphere cores to attain the target weight gain of approximately 5.94% w/w.
Process parameters for the drug layering process were as shown in Table 40.

TABLE 40
Drug layering process parameters - Example 16

| Operational Parameter | Target setpoint/range |
|---|---|
| Spray rate | 5-16 g/min |
| Nozzle air | 24-26 psi |
| Cylinder air | 19-21 psi |
| Inlet air volume | 40-45 cfm |
| Inlet air temperature | 35-38° C. |
| Product temperature | 25-28° C. |

### Seal Coating

1990 g of the naltrexone HCl cores were further coated with a polymeric dispersion from Table 41 using a VFC-Lab 3 FLO-COATER® equipped with a Wurster coating system. The process parameters were as shown in Table 42.

TABLE 41
Seal coating formulation composition - Example 16

| | | Quantity per |
|---|---|---|
| Component | Percent w/w (%) | batch (g) |
| Hydroxypropyl methylcellulose 5 | 3.75 | 52.5 |
| mpas (HPMC E5) | | |
| Talc | 1.25 | 17.5 |
| Ethanol 190 Proof | 95.0 | 1330.0 |
| Total | 100.0 | 1400.0 |

Seal Coating Process:
HPMC was dissolved in ethanol 190 proof using an overhead mixer until a clear solution was formed.
Talc was then added to the solution and mixed for no longer than 15 minutes.
1990 g of naltrexone HCl cores were charged into a VFC Lab-3 FLO-COATER.
The seal coating dispersion was layered onto the cores to attain a final weight gain of approximately 3.5% w/w.

TABLE 42
Seal coating process parameters - Example 16

| Operational Parameter | Target setpoint/range |
|---|---|
| Spray rate | 5-17 g/min |
| Nozzle air | 24-26 psi |
| Cylinder air | 19-21 psi |
| Inlet air volume | 40-42 cfm |
| Inlet air temperature | 40-42° C. |
| Product temperature | 27-30° C. |

### Delayed Release Layer

700 g of the naltrexone HCl seal coated cores were coated with an aqueous dispersion of enteric polymer (EUDRAGIT® L 30 D-55) using a VFC-Lab 1 FLO-COATER® equipped with a Wurster coating system. The composition and process of enteric coating was the same as for Example 14. The seal coated cores were coated to a target weight gain of 47% w/w and cured at 40° C. for 2 hours in a tray dryer.

### Example 17—Dissolution Comparison for Super Disintegrant (Croscarmellose Sodium) Level in Formulation

The dissolution profile of formulations with differing amounts of superdisintegrant (croscarmellose sodium) were compared. The dissolution profiles are shown in FIG. 3.

Dissolution method parameters:
Acid stage: 0.1N HCl; 750 mL, 0-120 minutes
Buffer stage: pH 5.5; 1000 mL, 120-200 minutes
Dissolution apparatus: USP II, Paddle
Agitation Speed: 50 RPM

At 30 minutes into the buffer stage, the formulation with a 100% relative amount of superdisintegrant was 94% dissolved, while the formulations with 50% and 0% relative amount of superdisintegrant were 86% dissolved.

For formulations with superdisintegrant, the in-vivo drug release was expected to be faster due to the inclusion of superdisintegrant in the formulation.

### Example 18—Low Dose Naltrexone HCl (LDN) DR Capsules, 2 mg Formulation

This example provides a Low Dose Naltrexone HCl (LDN) DR Capsules, 2 mg formulation

A multi-particulate system containing cores coated with an enteric delayed-release polymer was chosen as the formulation approach for LDN DR Capsules, 2 mg. The formulation design, manufacturing process and analytical results for the formulation were as follows.

### Manufacturing Process Overview

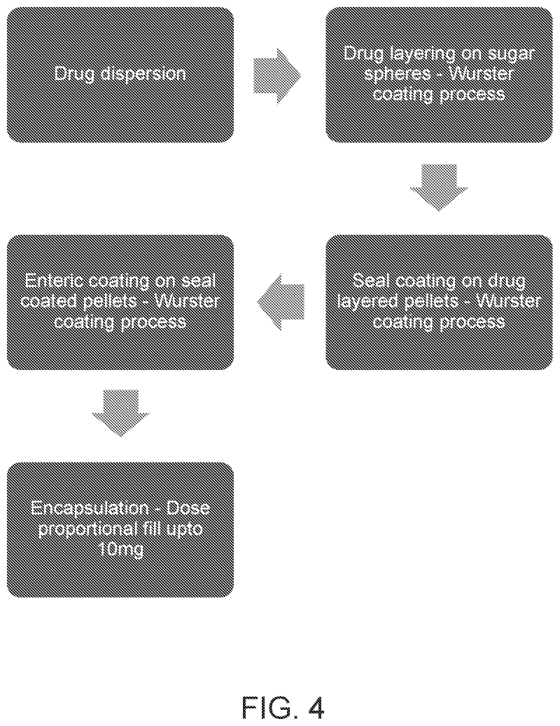

The manufacturing process for LDN DR capsules involved several steps outlined in the process flowchart in FIG. 4.

### Formulation and Process

The formulation for the drug layering (i.e. core production) process was as shown in Table 43.

TABLE 43
Formulation for naltrexone HCl cores - Example 18

| | | | Qty per |
|---|---|---|---|
| Material | Function | % w/w | batch (g) |
| Sugar Spheres 25/30 mesh | Inert Core | 95.67 | 3000.0 |
| Naltrexone HCl | Active | 2.55 | 79.9 |
| | Pharmaceutical | | |
| | ingredient | | |
| Hydroxypropyl | Binder | 0.25 | 8.0 |
| methylcellulose (HPMC | | | |
| E5) | | | |
| Talc | Anti-tacking agent | 0.25 | 8.0 |
| Croscarmellose sodium | Super disintegrant | 1.27 | 39.9 |
| Methanol | Solvent | N/A | 1714.0 |
| Purified water | Solvent | N/A | 90.2 |
| Total | N/A | 100.00 | 3135.8 |

### Manufacturing Process

A drug dispersion was prepared by dissolving HPMC E5 in a co-solvent mixture of methanol and purified water using an over-head mixer until a clear solution was formed. Naltrexone HCl was then added to the solution and mixed until a clear solution was obtained. Mixing was continued and talc was added and mixed for no longer than 15 minutes. Croscarmellose sodium was then added to the dispersion, and mixing was continued for no longer than 10 minutes.

The required quantity of sugar spheres was weighed and loaded into a Freund Vector VFC LAB-3 Flo Coater® equipped with a Wurster coating assembly. The sugar spheres were warmed to a product temperature of 26-28° C. and the drug dispersion was sprayed. Coating was performed at a rate of 7-17 g/min, maintaining a product temperature of 26-28° C. The cores produced were dried using a tray dryer at 60° C. for 8 hours to remove excess solvent.

### Seal Coating

The formulation for the seal coating process was as shown in Table 44.

TABLE 44
Formulation for naltrexone HCl seal coated cores - Example 18

| Material | Function | % w/w | Qty per batch (g) |
|---|---|---|---|
| Naltrexone HCl Cores | Core | 96.61 | 3000.00 |
| HPMC E5 | Polymer | 2.54 | 78.75 |
| Talc | Anti-tacking | 0.85 | 26.25 |
| | agent | | |
| Ethanol 190 proof | Solvent | N/A | 1995.00 |
| Total | N/A | 100.00 | 3105.0 |

### Manufacturing Process

A seal coating dispersion was prepared by dissolving HPMC E5 in 190 proof ethanol using an over-head mixer to obtain a clear solution. Talc was then added to the HPMC solution and mixed for no longer than 15 minutes. 3000 g of naltrexone HCl cores were loaded into a Freund Vector VFC LAB-3 Flo Coater® equipped with a Wurster coating assembly. The cores were warmed to a product temperature of 28-30° C., and the seal coating dispersion was sprayed on to the cores to attain a target weight gain of 3.5% w/w. The coating was performed at a rate of 7-20 g/min, maintaining a product temperature of 28-30° C. No additional drying was performed for the seal coated cores.

### Delayed-Release Coating

The formulation for the delayed-release coating process was as shown in Table 45.

TABLE 45
Formulation for naltrexone HCl DR pellets - Example 18

| | | | Qty per |
|---|---|---|---|
| Material | Function | % w/w | batch (g) |
| Naltrexone HCl Seal coated cores | Core | 68.0 | 700.0 |
| Methacrylic Acid and Ethyl | Delayed- | 20.0 | 685.4 |
| Acrylate Copolymer Dispersion | release | | |
| (EUDRAGIT ® L 30 D-55) | Polymer | | |
| Talc | Anti-tacking | 10.0 | 102.8 |
| | agent | | |
| Triethyl Citrate (TEC) | Plasticizer | 2.0 | 20.6 |
| Purified Water | Diluent | N/A | 836.2 |
| Total | N/A | 100.00 | 1645.0 |

### Manufacturing Process

TEC was dispersed in purified water using an over-head mixer, and mixing was performed until a homogenous dispersion was formed. Talc was then added and mixed for no longer than 15 minutes. The required quantity of EUDRAGIT® L 30 D-55 was dispensed in a separate beaker, and the TEC-Talc dispersion was slowly added to it while mixing using an over-head mixer. The final coating dispersion was mixed for no longer than 45 minutes.

700 g of Naltrexone HCl seal coated cores were loaded into a Freund Vector VFC LAB-1 Flo Coater® equipped with a Wurster coating assembly. The cores were warmed to a product temperature of no higher than 28-30° C. and the coating dispersion was sprayed. The coating was performed at a rate of 6-10 g/min, maintaining a product temperature of 28-30° C. to achieve a final weight gain of 47% w/w. The pellets were cured at 40° C. for 2 hours in a tray dryer.

### Encapsulation

The naltrexone HCl DR pellets were further encapsulated into Size 1 hard gelatin capsules.

### Analytical Testing and Results

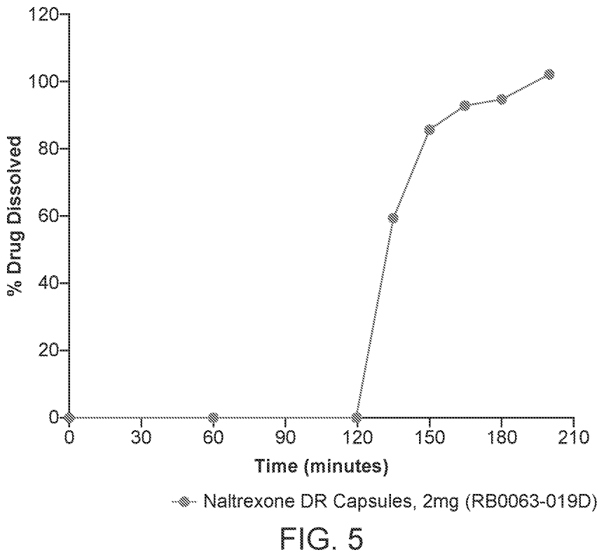

Dissolution testing was performed on the naltrexone HCl DR pellets using a two-stage dissolution method. The dissolution method and results are given in Table 46 and Table 47, respectively, and FIG. 5 depicts the dissolution profile. An Example dissolution test method protocol is given in Example 21.

TABLE 46
Dissolution method - Example 18
Dissolution method for Naltrexone HCl DR Capsules, 2 mg

| | |
|---|---|
| Method | Two-stage dissolution method |
| Apparatus | USP Apparatus II (Paddle) |
| Sinkers used | Yes |
| Temperature | 37° C. |
| Agitation Speed | 50 RPM |
| Stage 1 medium | 0.1N HCl |
| Stage 1 medium Volume | 750 mL |
| Stage 2 medium | pH 5.5 (Citrate buffer) |
| Stage 2 medium Volume | 1000 mL |
| Time in Acid stage | 2 hours (0-120 minutes) |
| Time in Buffer stage | 80 minutes (120-200 minutes) |

TABLE 47
Dissolution data for naltrexone HCl DR capsules, 2 mg - Example 18

| | % Drug Release | |
|---|---|---|
| Time (minutes) | (average of 3 units) | RSD |
| 0 | 0 | 0 |
| 60 | 0 | 0.0 |
| 120 | 0 | 0.0 |
| 135 | 60 | 7.3 |
| 150 | 86 | 3.1 |
| 165 | 94 | 2.2 |
| 180 | 95 | 1.6 |
| 200 | 103 | 0.9 |

### Discussion

The dissolution profile for the formulation of naltrexone HCl DR Capsules, 2 mg showed no release in 0.1N HCl (acidic medium) and a rapid release of more than 85% within 30 minutes in a pH of 5.5.

### Final Composition

The composition of naltrexone HCl DR Pellets, 2 mg in each unit operation was as shown in Table 48 and Table 49.

TABLE 48
Composition in mg/unit for naltrexone HCl DR pellets - Example 18

| | |
|---|---|
| Component | mg/unit |

Cores

| | |
|---|---|
| Sugar Spheres 25/30 mesh | 75.11 |
| Naltrexone HCl | 2.00 |
| HPMC E5 | 0.20 |
| Talc | 0.20 |
| Croscarmellose Sodium | 1.00 |
| Total | 78.51 |

Seal coating (3.5% w/w)

| | |
|---|---|
| HPMC E5 | 2.06 |
| Talc | 0.69 |
| Total | 81.26 |

Delayed-Release coating (47% w/w)

| | |
|---|---|
| EUDRAGIT ® L 30 D-55 | 23.87 |
| Talc | 11.94 |
| Triethyl Citrate (TEC) | 2.39 |
| Total Pellet Weight per Capsule | 119.46 |

TABLE 49
Final formulation composition per capsule for naltrexone HCl DR
capsules, 2 mg - Example 18

| Component | % w/w | mg/unit |
|---|---|---|
| Sugar Spheres 25/30 mesh | 62.9 | 75.11 |
| Naltrexone HCl | 1.7 | 2.00 |
| HPMC E5 | 1.9 | 2.26 |
| Talc | 10.7 | 12.83 |
| Croscarmellose sodium | 0.8 | 1.00 |
| EUDRAGIT ® L 30 D-55 | 20.0 | 23.87 |
| Triethyl Citrate (TEC) | 2.0 | 2.39 |
| Total | 100.0 | 119.46 |

### Example 19—Dissolution Comparison for Naltrexone HCl DR Capsules, 4.5 mg in Different Buffer Stages

The naltrexone HCl DR pellets from Example 18 were encapsulated into Size 1 hard gelatin capsules such that the capsules contained 4.5 mg of naltrexone HCl.

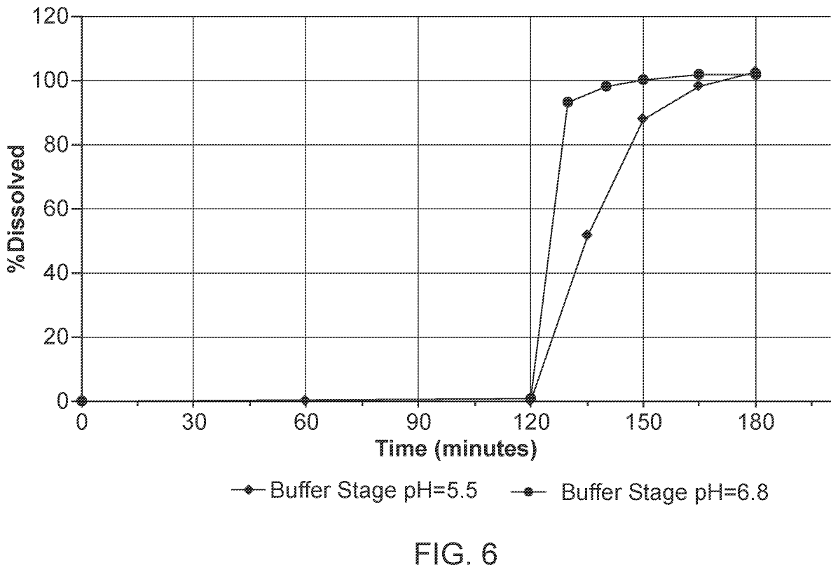

Dissolution testing was performed on 4.5 mg naltrexone HCl DR pellets using a two-stage dissolution method. The method was similar to that used in Example 18, except a pH 6.8 buffer stage was tested in addition to the pH 5.5 buffer stage. The dissolution profile is shown in FIG. 6.

The dissolution test employed the parameters:
Acid stage: 0.1N HCl; 750 mL, 0-120 minutes
Buffer stage: pH 5.5; 1000 mL, 120-180 minutes OR pH 6.8; 1000 mL, 120-180 minutes
Dissolution apparatus: USP II, Paddle
Agitation Speed: 50 RPM

The tests showed≤1% dissolution after 120 minutes in the acid stage. The dissolution test measured 52%, 88%, 98%, and 102% at 15, 30, 45, and 60 minutes in the pH 5.5 buffer, respectively. The dissolution test measured 93%, 98%, 100%, 102%, and 102% dissolution at 10, 20, 30, 45, and 60 minutes in the pH 6.8 buffer, respectively.

### Example 20—Naltrexone HCl Excipient Compatibility Study

An excipient compatibility study was designed and conducted to screen excipients for the low dose naltrexone HCl delayed release capsules for formulation development purposes. Based on the formulation approach of a multi-particulate system filled in capsule dosage form, the drug layered spheres were coated with a functional pH dependent polymer film. Table 50 shows the list of materials selected for the study and their functions. Table 51 shows the drug/excipient ratios used in the excipient compatibility study.

TABLE 50
List of material - Example 20

| Material | Function | Manufacturer/ Lot# |
|---|---|---|
| Naltrexone Hydrochloride USP | Active Pharmaceutical | SpecGx LLC/2007000305 |
| | Ingredient | |
| Hydroxypropyl methylcellulose | Binder/polymer | DuPont/D011G48L01 |
| (HPMC E5) | | |
| PVP K30 (Kollidon ® 30) | Binder/polymer | BASF/G02167PTO |
| Croscarmellose Sodium (Ac-di- | Disintegrant | DuPont/TN18832540 |
| sol) | | |
| Crospovidone (Kollidon CL-F) | Super-disintegrant | BASF/01644729U0 |
| Methacrylic Acid and Methyl | Delayed release polymer | Evonik/B190303201 |
| Methacrylate Copolymer (1:1) - | | |
| NF (EUDRAGIT ® L 100) | | |
| Methacrylic Acid and Ethyl | Delayed release polymer | Evonik/C191114639 |
| Acrylate Copolymer Dispersion - | | |
| NF (EUDRAGIT ® L 30 D-55) | | |
| Talc | Glidant/Anti-static agent | Barretts Minerals |
| | | Inc./B0145N1 |
| Silica - Silicon Dioxide | Glidant/Anti-static agent | Grace/5210187201 |
| (SYLOID ® 244FP) | | |

TABLE 51
Excipient compatibility study design - Example 20

| | Naltrexone | HPMC | PVP | Croscarmellose | | EUDRAGIT ® | EUDRAGIT ® | | |
|---|---|---|---|---|---|---|---|---|---|
| | HCl | E5 | K30 | Sodium | Crospovidone | L 100 | L 30 D-55 | Talc | Silica |
| Naltrexone | 1 | | | | | | | | |
| HCl | | 1:2 | | | | | | | |
| | | | 1:2 | | | | | | |
| | | | | 1:2 | | | | | |
| | | | | | 1:2 | | | | |
| | | | | | | 1:5 | | | |
| | | | | | | | 1:5 | | |
| | | | | | | | | 1:5 | |
| | | | | | | | | | 1:5 |

Additionally, control samples were prepared for all inactive ingredients and were tested as needed. The physical mixtures were prepared by weighing individual material into glass crimp vials and mixing using a vortex. The open samples were left open and closed samples were tightly closed using a crimper. Table 52 lists stability time points and storage conditions.

TABLE 52
Excipient compatibility study conditions and time points - Example 20

| | | No. of Sample |
|---|---|---|
| Stability Condition | Time points | Sets |
| Initial | N/A | 1 |
| 40° C./ 75% RH - Open | 2 Weeks | 2 |
| | 2 Months | 2 |
| 40° C./ 75% RH - Closed | 2 Weeks | 2 |
| | 2 Months | 2 |
| | 3 Months | 1 |

### Results

The initial and 2-week samples were tested, and results are given in Table 53.

TABLE 53
Assay and impurities - Example 20

| | Sample Stability Time points and Storage Condition | | | | | |
|---|---|---|---|---|---|---|
| | | | 40° C./ 75% RH - | | 40° C./ 75% RH - | |
| | Initial | | Open - 2 weeks | | Closed - 2 weeks | |
| | Assay | Impurities | Assay | Impurities | Assay | Impurities |
| | (%) | (%) | (%) | (%) | (%) | (%) |
| Naltrexone Control | 99.4 | 0.4 | 101.5 | 0.6 | 99.3 | 0.6 |
| Naltrexone + HPMC E5 | 99.0 | 0.5 | 98.5 | 1.0 | 99.3 | 0.3 |
| Naltrexone + PVPK30 | 99.4 | 0.4 | 98.4 | 1.3 | 99.5 | 0.3 |
| Naltrexone + | 99.1 | 0.4 | 98.6 | 0.6 | 99.1 | 0.4 |
| Croscarmellose Sodium | | | | | | |
| Naltrexone + | 99.3 | 0.4 | 99.4 | 0.9 | 100.4 | 0.4 |
| Crospovidone | | | | | | |
| Naltrexone + | 99.4 | 0.4 | 99.5 | 2.0 | 99.8 | 0.4 |
| EUDRAGIT ® L 100 | | | | | | |
| Naltrexone + | 97.2 | 0.4 | 93.3 | 2.8 | 96.0 | 2.9 |
| EUDRAGIT ® L 30 D- | | | | | | |
| 55 | | | | | | |
| Naltrexone + Talc | 98.5 | 0.3 | 98.1 | 0.7 | 98.2 | 0.3 |
| Silica - Silicon Dioxide | 98.3 | 0.4 | 99.0 | 0.7 | 98.9 | 0.3 |
| (SYLOID ® 244FP) | | | | | | |

### Discussion

From the test results listed in Table 53, no significant change in assay or impurities was observed in samples other than the samples containing a physical mixture of naltrexone HCl and EUDRAGIT® L 30 D-55. The samples containing naltrexone HCl and EUDRAGIT® L 30 D-55 showed lower assay and higher impurities at the 2-week timepoint in both open and closed condition samples, indicating an interaction between the drug and the polymer and possible degradation. The stressed stability results under 2-week open and closed conditions provided meaningful information to proceed to formulation development, however the study was further monitored for all the samples for up to 3 months.

### Example 21—Dissolution Test Method

### 1.0 Purpose

This example provides a test method for dissolution testing of delayed-release capsules. This dissolution test method was employed in various examples.

Separation was achieved by using a Zorbax Extend C18, 3.5 μm, 4.6×100 mm column and isocratic elution. Detection was by HPLC at 280 nm and quantitation was done by comparing the peak response of naltrexone in the sample with external standards.

### 2.0 Scope

2.1 This procedure applied to the analysis of, for example, the following samples: Naltrexone Hydrochloride Delayed-Release Capsules.

### 3.0 Reference Standard

3.1 Naltrexone Hydrochloride API
Instructions for Use: Followed the instructions on the label text of Certificate.

### 4.0 Reagents and Equipment

4.1 Instrumentation and Equipment

Distek Dissolution tester with Auto-sampler
Waters ACQUITY UPLC H-Class, ACQUITY Arc or Equivalent
Analytical balance: Capable of measuring to 0.01 mg
Ultrasonic Bath

4.2 Reagents and Chemicals

Hydrochloric Acid, A.C.S. Reagent or equivalent
Sodium Phosphate Tribasic, A.C.S. Reagent or equivalent
Ammonium Bicarbonate, A.C.S. Reagent or equivalent
Acetonitrile, HPLC Grade or equivalent
Water, Purified by Milli-Q System or HPLC Grade

### 5.0 Procedure: Dissolution Testing

5.1 Preparation of Dissolution Medium

5.1.1 Dissolution Medium: 0.1 N Hydrochloric Acid

Transfer 50 mL of Hydrochloric Acid into 5 L of Water, QS to 6 L with Water, and mix well. Degas prior to use.

The Dissolution Medium may be scaled proportionately as needed.

Procedure for degassing: Heat the dissolution medium to 41° C.-45° C., and perform a vacuum filtration through a 0.45 μm filter while stirring. Continue to pull vacuum for not less than 5 additional minutes.

5.1.2 Dissolution Medium: 0.2M Citrate Buffer

Accurately weigh 165.47 g of sodium citrate dehydrate, 45.6 g of citric acid and 56 g of sodium hydroxide and dissolve in 4 L of water. Filter through a 0.45 μm membrane filter and degas.

5.2 Dissolution Testing Conditions

| | |
|---|---|
| Apparatus: | USP Apparatus II (Paddle) with Sinker |
| Dissolution Medium: | Acid Stage: 0.1N Hydrochloric acid, 750 mL |
| | Buffer Stage: pH 5.5 Citrate Buffer, 1000 mL |
| Agitation Speed: | 50 rpm |
| The distance between the | 25 ± 2 mm |
| inside bottom of the vessel | |
| and the bottom of the basket | |
| Time Intervals: | Acid Stage: 60, 120 minutes |
| | Buffer Stage: 15, 30, 45 and 60 minutes |
| Temperature: | 37° C. ± 0.5° C. |
| Sampling Volume: | 5 mL for manual and 1.3 mL for autosampler |

5.3 Dissolution Testing Procedure:

A) Acid Stage

Place 750 mL of 0.1N HCl into the dissolution vessels and assemble the apparatus. Set the dissolution tester temperature to 37.0° C. and set the agitation speed to 50 rpm. Allow the dissolution medium to equilibrate to a temperature of 37±0.5° C. prior to the start of dissolution. Set the auto-sampler program to collect sample solutions at the specified time intervals. Check the temperature in each dissolution vessel using a calibrated thermometer to ensure that a temperature of 37±0.5° C. is reached. Weigh each Capsule individually and record the tablet weights. Place the Capsule samples into vessel and start to run the apparatus at the specified rate. At the end of each time interval, withdraw 5 mL of sample solution from each vessel.

B I) Buffer Stage (pH 5.5):

Equilibrate the citrate buffer to 37±0.5° C. Check the temperature in each dissolution vessel using a calibrated thermometer to ensure that a temperature of 37±0.5° C.

After the 2-hour time point sampling in the acid stage, immediately add 250 mL of the pre-equilibrated citrate buffer to each vessel. Continue the dissolution and collect samples at each time interval, withdraw 5 mL of sample solution from each vessel.

B II) Buffer Stage (pH 6.8):

In some examples, the buffer stage employed a pH 6.8 phosphate buffer prepared by analogous methods as for the pH 5.5 citrate buffer.

Equilibrate the phosphate buffer to 37±0.5° C. Check the temperature in each dissolution vessel using a calibrated thermometer to ensure that a temperature of 37±0.5° C.

After the 2-hour time point sampling in the acid stage, immediately add 250 mL of the pre-equilibrated phosphate buffer to each vessel. Continue the dissolution and collect samples at each time interval, withdraw 5 mL of sample solution from each vessel.

### 6.0 Procedure: Dissolution Sample Analysis

6.1 Preparation of pH 10 Buffer

Accurately weigh about 4.8 g of Ammonium Carbonate and dissolve in 1000 mL of water. Adjust pH to 10.0±0.1 with 10N Sodium Hydroxide. Filter through a 0.2 μm membrane filter.

The buffer may be scaled proportionately as needed.

6.2 Preparation of Mobile Phase

Mix the pH 10.0 buffer with Acetonitrile in a ratio of 40:60 (v/v). Degas with sonication for at least 5 minutes before use.

6.3 Preparation of Diluent

Mix 0.1N HCl and 0.2M Sodium Phosphate Tribasic buffer in a ratio of 3:1 (v/v).

6.4 Preparation of Standard Stock Solution

Standard Solution: 0.22 mg/mL of Naltrexone Hydrochloride Reference Standard in Diluent.

Accurately weigh about 22 mg of Naltrexone Hydrochloride Reference Standard and transfer it into a 100 mL volumetric flask. Add about 70 mL of Diluent and sonicate until the Reference Standard is completely dissolved. Dilute to volume with Diluent and mix well.

Prepare two separate Standard Stock Solutions.

6.5 Preparation of Intermediate Standard Solution
Intermediate Standard Solution: 0.022 mg/mL of Naltrexone Hydrochloride Reference Standard in Diluent.

Pipette 5.0 mL of the Standard Stock Solution into a 50 mL volumetric flask. Dilute to volume with Diluent and mix well.

Prepare two separate Intermediate Standard Solutions and label one as Standard Solution and the other one as Check Standard Solution.

6.6 Preparation of Standard Solution
Standard Solution: 0.0022 mg/mL of Naltrexone Hydrochloride Reference Standard in Diluent.

Pipette 5.0 mL of the Intermediate Standard Solution into a 50 mL volumetric flask. Dilute to volume with Diluent and mix well.

Prepare two separate Standard Solutions and label one as Standard Solution and the other one as Check Standard Solution.

6.7 Preparation of Sample Solution

Filter a portion of the Dissolution sample solution through a 0.45 μm pore size Nylon syringe filter. Discard 2 mL of the initial filtrate and collect the filtrate in an LC sample vial for analysis.

6.8 Chromatographic Conditions

| | |
|---|---|
| Instrument | Waters ACQUITY UPLC H-Class, ACQUITY |
| | Arc or Equivalent |
| LC Column | Zobax Extend C18, 3.5 μm, 4.6 × 100 mm |
| Detector Wavelength | 280 nm |
| Column Temperature | 35° C. |
| Flow Rate | 0.5 mL/min |
| Injection Volume | 50 μL |
| Run Time | 5 minutes |

6.9 Example Injection Sequence

| | |
|---|---|
| Description | Number of Injections |

Equilibrate the system with Mobile Phase until a stable baseline has been
achieved

| | |
|---|---|
| Dissolution Medium | One injection |
| System Suitability Solution | Five Replicate injections |
| Check Standard Solution | Duplicate injections |
| Sample Solution | Single injection per preparation, up to 12 |
| | injections |
| Standard Solution | One injection |

Continue with Sample and Bracketing Standard injections as needed.

6.10 System Suitability Testing Requirements

| | |
|---|---|
| System Precision | The percent relative standard deviation (% RSD) |
| | of the peak area of Naltrexone from 5 replicate |
| | injections of System Suitability Solution should be |
| | NMT 2.0%. |
| Overall Precision | The percent relative standard deviation (% RSD) |
| | of the peak area of Naltrexone for all the Standard |
| | Solution injections over the run should be NMT |
| | 2.0%. |
| Response Factor | The ratio of the response factor for the Standard |
| | Solution and the Check Standard Solution should |
| | be within 0.980-1.020. |
| Tailing Factor | The tailing factor for Naltrexone from System |
| | Suitability Solution should be NMT 2.0. |
| Plate Count | The plate count of Naltrexone in the standard |
| | injections should be no larger than 3000. |

### 7.0 Calculations

7.1 Ratio of Response Factor Calculation:

$$Ratio of Response Factor = \frac{Ac}{CStd . Wt .} \times \frac{WStd . Wt .}{As}$$

Where,

| | | |
|---|---|---|
| As | = | Average peak area of Naltrexone from the Standard Solution |
| Ac | = | Average peak area of Naltrexone from the Check Standard |
| | | Solution |
| WStd. Wt. | = | Weight of Naltrexone in the Standard Solution preparation, in mg |
| CStd. Wt. | = | Weight of Naltrexone in the Check Standard Solution |
| | | preparation, in mg |

7.2 Calculation for Naltrexone Release (%) for Finished Products

7.2.1 Naltrexone Release (%) at 60-min Interval:

$$= \frac{Au}{As} \times \frac{Std . Wt .}{50 mL} \times \frac{5. mL}{100 mL} \times P \times 750 mL \times \frac{1}{LC} \times 100$$

7.2.2 Naltrexone Release (%) at 120-min Interval:

$$= \{ \frac{Au}{As} \times \frac{Std . Wt .}{50 mL} \times \frac{5. mL}{100 mL} \times P \times 745 mL + W 1 \} \times \frac{1}{LC} \times 100$$

7.2.3 Naltrexone Release (%) at 135-min Interval:

$$= \{ \frac{Au}{As} \times \frac{Std . Wt .}{50 mL} \times \frac{5. mL}{100 mL} \times P \times 990 mL + W 1 + W 2 \} \times \frac{1}{LC} \times 100$$

7.2.4 Naltrexone Release (%) at 150-min Interval:

$$= \{ \frac{Au}{As} \times \frac{Std . Wt .}{50 mL} \times \frac{5. mL}{100 mL} \times P \times 985 mL + W 1 + W 2 + W 3 \} \times \frac{1}{LC} \times 100$$

7.2.5 Naltrexone Release (%) at 165-min Interval:

$$= \{ \frac{Au}{As} \times \frac{Std . Wt .}{50 mL} \times \frac{5. mL}{100 mL} \times P \times 980 mL + W 1 + W 2 + W 3 + W 4 \} \times \frac{1}{LC} \times 100$$

Naltrexone Release (%) at 180-min Interval:

$$= \{ \frac{Au}{As} \times \frac{Std . Wt .}{50 mL} \times \frac{5. mL}{100 mL} \times P \times 975 mL + W 1 + W 2 + W 3 + W 4 + W 5 \} \times \frac{1}{LC} \times 100$$

Where,
Au=Peak area of Naltrexone from the Sample Solution
As=Average Peak area of Naltrexone from the Standard Solution
Std.Wt.=Weight of Naltrexone Reference Standard in the Standard Solution preparation, in mg
P=Purity factor of Naltrexone Reference Standard
LC=Label Claim of Naltrexone, 0.2 mg
W1=Amount of Naltrexone withdrawn at 60 min Interval, in mg
W2=Amount of Naltrexone withdrawn at 120 min Interval, in mg
W3=Amount of Naltrexone withdrawn at 135 min Interval, in mg
W4=Amount of Naltrexone withdrawn at 150 min Interval, in mg
W5=Amount of Naltrexone withdrawn at 165 min Interval, in mg

### Example 22—Naloxone HCl Delayed Burst Formulation

This example provides a naloxone HCl delayed burst formulation which had a core composition according to Table 54.

TABLE 54
Core composition-Example 22

| Component | Percent w/w (%) | Quantity per batch (g) |
|---|---|---|
| Naloxone HCl | 15.0 | 120.0 |
| Microcrystalline cellulose | 75.0 | 600.0 |
| (MCC) | | |
| Crospovidone | 5.0 | 40.0 |
| Hydroxypropyl | 5.0 | 40.0 |
| methylcellulose (HPMC) | | |
| 5 mpas | | |
| Total | 100.0 | 800.0 |

The manufacturing process:
Naloxone, MCC and crospovidone were screened through a mesh #40 sieve to de-agglomerate.
The deagglomerated material was charged into a high shear granulator and mixed to form a mixture.
HPMC was dissolved in purified water to prepare a granulation aid.
The mixture was granulated using the granulation aid until granule formation was observed; process parameters were as shown in Table 55.
The naloxone granules were milled if necessary and dried using a tray drying or a fluid bed processor.
The naloxone granules were milled using a suitable screen. A subcoat was added to the naloxone granules using the enteric polymer coating composition given in Table 56.

TABLE 55
Granulation process parameters-Example 22

| | Operational | Target |
|---|---|---|
| Operation | Parameter | setpoint |
| Wet Granulation | Impeller speed | 350 RPM |
| | Chopper speed | 1000 RPM |
| | Spray rate | 15-20 g/min |
| Drying | Temperature | 55° C. |

### Outer Enteric Delayed Release Polymer Coating

The enteric polymer coating dispersion from Table 56 in a suitable solvent system was sprayed onto 700 g of naloxone granules using a fluid bed processor equipped with a Wurster column. The coating parameters are given in Table 57. Samples were collected at different coating weight gains and dissolution was performed to evaluate the target coating level.

TABLE 56
Outer delayed release coating composition-Example 22

| Component | Percent w/w (%) |
|---|---|
| EUDRAGIT ® L 100 | 58.8 |
| Triethyl Citrate (TEC) | 11.8 |
| Talc | 29.4 |
| Total | 100.0 |

TABLE 57
Coating process parameters-Example 22

| Operational Parameter | Target setpoint/range |
|---|---|
| Inlet air volume | 40-50 cfm |
| Inlet air temperature | 30-45° C. |
| Column height | 0.5″ |
| Nozzle air | 20-25 psi |
| Accelerator air | 15-20 psi |
| Solution spray rate | 6-8 g/min |

### Example 23—Naloxone HCl Delayed Burst Formulation

This example provides a naloxone HCl delayed burst formulation.

TABLE 58
Core composition-Example 23

| Component | Percent w/w (%) | Quantity per batch (g) |
|---|---|---|
| Naloxone HCl | 12.5 | 100.0 |
| Microcrystalline cellulose | 77.5 | 620.0 |
| (MCC) | | |
| Sodium carboxymethyl | 5.0 | 40.0 |
| cellulose (Sodium CMC) | | |
| Hydroxypropyl cellulose | 5.0 | 40.0 |
| (HPC) | | |
| Total | 100.0 | 800.0 |

Manufacturing Process:
Naloxone, MCC and sodium CMC were screened through a mesh #40 sieve to de-agglomerate.
The material was charged into a high shear granulator and mixed.
HPC was dissolved in purified water to prepare the granulation aid.
The mixture was granulated using the granulation aid until granule formation was observed.
The granules were then passed through an extruder followed by spheronization to form naloxone cores.
Naloxone cores were further dried using a tray dryer or a fluid bed processor.
Enteric polymer coating was performed on the naloxone cores using the membrane coating composition of Table 59.

### Outer Enteric Delayed Release Coating

The outer enteric delayed release coating dispersion from Table 59 in a suitable solvent system was sprayed on to 700 g of naloxone granules using a fluid bed processor equipped with a Wurster column. The coating parameters are given in Table 60. Samples were collected at different coating weight gains and dissolution was performed to evaluate the target coating level.

TABLE 59
Delayed release coating composition-Example 23

| Component | Percent w/w (%) |
|---|---|
| EUDRAGIT ® L 100-55 | 62.5 |
| Triethyl Citrate (TEC) | 6.3 |
| Talc | 31.2 |
| Total | 100.0 |

TABLE 60
Coating process parameters-Example 23

| Operational Parameter | Target setpoint/range |
|---|---|
| Inlet air volume | 40-50 cfm |
| Inlet air temperature | 30-45° C. |
| Column height | 0.5″ |
| Nozzle air | 23-30 psi |
| Accelerator air | 18-25 psi |
| Solution spray rate | 6-8 g/min |

### Example 24—Naloxone HCl Delayed Burst Formulation

This example provides a naloxone HCl delayed burst formulation with a core composition according to Table 61.

TABLE 61
Core composition-Example 24

| Component | Percent w/w (%) | Quantity per batch (g) |
|---|---|---|
| Naloxone HC1 | 5.0 | 250.0 |
| Hydroxypropyl | 0.5 | 25.0 |
| methylcellulose (HPMC) | | |
| 5 mpas | | |
| Talc | 0.8 | 150.0 |
| Ethanol | 65.6 | 3280.0 |
| Purified Water | 28.1 | 1405.0 |
| Total | 100.0 | 5000.0 |

Manufacturing Process:
Naloxone and HPMC were dissolved in an ethanol-water cosolvent system.
Talc as added to the solution and mixed for no longer than 15 mins
2000 g of sugar spheres were charged into the rotor granulator bowl.
The drug dispersion was layered onto the sugar sphere cores to attain the target weight gain. Process parameters for the drug layering process were as shown in Table 62.
The cores were dried using a tray dryer.

TABLE 62
Drug layering process parameters-Example 24

| Operational Parameter | Target setpoint/range |
|---|---|
| Spray rate | 5-22 g/min |
| Nozzle air | 25-32 psi |
| Drying air flow | 22-28 cfm |
| Drying air temperature | 45-60° C. |
| Product temperature | 24-32° C. |
| Slit airflow | 12-16 cfm |
| Slit air temperature | 51-58° C. |
| Rotor speed | 200-320 rpm |

### Disintegrant Layering

A layer of disintegrant, sodium carboxymethyl cellulose, was applied in the form of a dispersion onto the naloxone cores. Coating was performed in a fluid bed processor equipped with a Wurster column using the disintegrant dispersion composition from Table 63 in a suitable solvent system on 700 g of naloxone cores. Coating was performed to a target weight gain of 5% w/w.

TABLE 63
Coating composition-Example 24

| Component | Percent w/w (%) |
|---|---|
| Sodium carboxymethyl | 7.5 |
| cellulose (Croscarmellose | |
| sodium) | |
| Hydroxypropyl | 1.0 |
| methylcellulose (HPMC) E5 | |
| Talc | 1.5 |
| Ethanol 190 Proof | 90.0 |
| Total | 100.0 |

### Example 25—Naloxone HCl Delayed Burst Formulation

This example provided a naloxone HCl delayed burst formulation with a core composition according to Table 64.

TABLE 64
Core composition-Example 25

| Component | Percent w/w (%) | Quantity per batch (g) |
|---|---|---|
| Naloxone HCl | 11.0 | 220.0 |
| Microcrystalline Cellulose | 65.0 | 1300.0 |
| (MCC) | | |
| Crospovidone | 8.0 | 160.0 |
| Talc | 10.7 | 214.0 |
| Silicon Dioxide | 0.3 | 6.0 |
| Polyvinyl pyrrolidine (PVP) | 5.0 | 100.0 |
| Total | 100.0 | 2000.0 |

Manufacturing Process:
Naloxone, MCC, crospovidone and talc were screened through a mesh #40 sieve to de-agglomerate and charged into a v-blender.
The materials were mixed for 10 minutes.
Silicon dioxide was screened through a mesh #40 sieve to de-agglomerate and charged into a v-blender.
The material was mixed for 5 minutes and the blend discharged.
The blend was then charged into the rotor granulator bowl.
PVP was dissolved in ethanol-water cosolvent system using an overhead mixer.
The blend was granulated in the rotor granulator using the PVP solution to form spherical drug cores.
The drug cores were dried in a tray dryer.

TABLE 65
Drug layering process parameters-Example 25

| Operational Parameter | Target setpoint/range |
|---|---|
| Spray rate | 5-22 g/min |
| Nozzle air | 25-32 psi |
| Drying air flow | 10-20 cfm |
| Drying air temperature | 20-30° C. |
| Product temperature | 10-20° C. |
| Slit airflow | 5-10 cfm |
| Slit air temperature | 20-30° C. |
| Rotor speed | 200-400 rpm |

### Prophetic Example 26—PK Study of 4.5 mg Naltrexone HCl DR Capsules

An open-label, 3-period, 3-treatment, randomized study is to be conducted to characterize the PK and safety and tolerability of 4.5 mg Naltrexone HCl DR Capsules under fasting and fed conditions and to compare to the PK of Naltrexone HCl Tablets, USP in healthy adult subjects. The study consists of an up-to-28-day screening period, three single-dose treatment periods, each consisting of two-night inpatient stays at the clinical research unit (CRU) and four outpatient visits, with a 7-day washout period between each. Study objectives are to characterize the single-dose pharmacokinetics (PK) of naltrexone and metabolite (6β-naltrexol) following administration of 4.5 mg Naltrexone HCl DR Capsules compared to Naltrexone Hydrochloride Immediate Release (IR) Tablets, USP (50 mg naltrexone HCl) in healthy adult subjects under fasting condition; to characterize the effect of food intake on the PK of naltrexone and 6β-naltrexol following administration of 4.5 mg Naltrexone HCl DR Capsules in healthy adult subjects; and to evaluate the safety and tolerability of single doses of 4.5 mg Naltrexone HCl DR Capsules under fed and fasting conditions compared to Naltrexone HCl IR Tablets, USP (50 mg naltrexone HCl) in healthy adult subjects. Approximately 18 subjects are to be enrolled in order to achieve approximately 12 evaluable subjects in each treatment period who will complete all 3 treatment periods.

### Prophetic Example 27—Safety and Tolerability Study of 4.5 mg Naltrexone HCl DR Capsules

A double-blind, randomized, two-treatment, two-sequence, two-period, repeat-dose crossover study is to be conducted to characterize the safety and tolerability of 4.5 mg Naltrexone HCl DR Capsules compared to naltrexone HCl IR capsules 4.5 mg in healthy adult subjects. The study will consist of an up to 28-day screening period and two 1-week treatment periods, each consisting of once-a-day (QD) dosing over 3 days followed by a 4-day washout period. Approximately 52 subjects are to be randomized using a 1:1 allocation to treatment sequence.

### Prophetic Example 28—Exploratory Trial of 4.5 mg Naltrexone HCl DR Capsules for Long-Covid

A 16-week, randomized, double blind, placebo-controlled trial is to be conducted to evaluate the safety and efficacy of low dose naltrexone versus placebo in reducing pain and/or one or more symptoms related to Post-Covid-19 syndrome in forty adults with Post-Covid-19 syndrome. The study will be a crossover design. Patients will age assigned to one of two groups:
Sequence 1: treatment with DBR-LDN for two months, followed by treatment with placebo for another two months.
Sequence 2: treatment with placebo for two months, followed by treatment with DBR-LDN for another two months.

The trial may be an open label single arm trial.

### Embodiments

### Embodiments I

Embodiment I-1. An oral delayed burst formulation of low-dose naltrexone (LDN) comprising a low-dose naltrexone and pharmaceutical excipients core, a subcoat surrounding the core comprising at least one water soluble hydrophilic carrier and an outer coating.

Embodiment I-2. The oral delayed burst formulation of low-dose naltrexone (LDN) of Embodiment I-1, wherein the core is in the form of a tablet.

Embodiment I-3. The oral delayed burst formulation of low-dose naltrexone (LDN) of Embodiment I-1, wherein an enteric delayed-release coating releases a low-dose naltrexone mixture with a lag time of one to three hours after direct contact with a body fluid.

Embodiment I-4. A method for treating a subject with low-dose naltrexone, comprising administering to the subject a formulation having a therapeutically effective amount of low dose naltrexone or a pharmaceutically acceptable salt thereof, wherein the formulation provides a delayed burst release after one to three hours resulting in dispersion mainly through the small intestine of the active ingredient into the blood stream.

Embodiment I-5. A delayed-release coating comprising a water insoluble capsule body closed at one end with an insoluble, but permeable and swellable hydrogel plug, wherein the plug comprises a material selected from the group consisting of polymethacrylates, erodible compressed polymers, congealed melted polymer and enzymatically controlled erodible polymers.

Embodiment I-6. A delayed burst release oral formulation of a low-dose naltrexone (LDN) or a pharmaceutically acceptable salt or ester thereof in the gastrointestinal tract of a subject, comprising:
(a) a core comprising low-dose naltrexone (LDN), and at least one burst controlling agent, wherein the burst controlling agent is a water insoluble polymer;
(b) a subcoat surrounding the core comprising at least one water soluble hydrophilic carrier; and
(c) an outer coating over the core, the outer coating comprising a water insoluble hydrophobic carrier and a water insoluble hydrophilic particulate matter, the water insoluble hydrophilic particulate matter allowing entry of liquid into the core.

Embodiment I-7. The delayed burst release oral formulation of a low-dose naltrexone (LDN) of Embodiment I-6, wherein the outer coating comprises a combination of at least one swellable polymer and at least one water insoluble polymer.

Embodiment I-8. The delayed burst release oral formulation of a low-dose naltrexone (LDN) of Embodiment I-6, wherein the outer coating is a two-layered coating comprising a rupturing outer layer and swellable inner layer.

Embodiment I-9. The delayed burst release oral formulation of a low-dose naltrexone (LDN) of Embodiment I-8, wherein the outer coaling further comprises a surfactant.

Embodiment I-10. The delayed burst release oral formulation of a low-dose naltrexone (LDN) of Embodiment I-9, wherein the surfactant in the outer coating is sodium lauryl sulfate (SLS).

Embodiment I-11. The delayed burst release oral formulation of a low-dose naltrexone (LDN) of Embodiment I-6, wherein the water soluble hydrophilic carrier of the subcoat is selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose HPMC, carboxy methyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, water soluble gum, polysaccharide, hydroxypropyl methylcellulose phthalate, polyvinyl acetate phthalate, cellulose acetate phthalate, hydroxypropyl methylcellulose acetate succinate, poly(methacrylic acid, methyl methacrylate)1:1, poly(methacrylic acid, ethyl acrylate)1:1, alginic acid, and sodium alginate, and any other pharmaceutically acceptable polymer that dissolves in phosphate buffer pH>5.5 or mixtures thereof.

Embodiment I-12. The delayed burst release oral formulation release of a low-dose naltrexone (LDN) of Embodiment I-11, wherein the water soluble hydrophilic carrier is polyvinyl pyrrolidone.

Embodiment I-13. The delayed burst release oral formulation of a low-dose naltrexone (LDN) of Embodiment I-6, wherein the subcoat further comprises at least one water insoluble particulate matter, wherein the water insoluble particulate matter is selected from the group consisting of microcrystalline cellulose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide and cross-linked polyacrylic acid.

Embodiment I-14. The delayed burst release oral formulation for localized release of a low-dose naltrexone (LDN) of Embodiment I-13, wherein the water insoluble particulate matter is microcrystalline cellulose.

Embodiment I-15. The delayed burst release oral formulation of a low-dose naltrexone (LDN) of Embodiment I-13, wherein the water-soluble hydrophilic carrier of the subcoat is a combination of povidone and microcrystalline cellulose.

Embodiment I-16. The delayed burst release oral formulation of a low-dose naltrexone (LDN) of Embodiment I-6, wherein at least about 60% of the naltrexone is released about 1 hour after the delayed burst release occurs.

Embodiment I-17. The delayed burst release oral formulation of a low-dose naltrexone (LDN) of Embodiment I-6, wherein the water insoluble hydrophilic particulate matter forms channels in the outer coating upon contact with a liquid, whereby the channels absorb body liquid and cause at least one burst controlling agent to burst the coating, thereby providing delayed burst release of the naltrexone.

Embodiment I-18. The delayed burst release oral formulation of a low-dose naltrexone (LDN) of Embodiment I-6, wherein the cross-linked polysaccharide is selected from the group consisting of insoluble metal salts or cross-linked derivatives of alginate, pectin, xanthan gum, guar gum, tragacanth gum, locust bean gum, and carrageenan.

Embodiment I-19. The delayed burst release oral formulation of a low-dose naltrexone (LDN) of Embodiment I-18, wherein the modified cellulose is selected from the group consisting of cross-linked derivatives of hydroxypropylcellulose, hydroxypropyl methylcellulose, hydroxyethyl cellulose, methylcellulose, carboxymethylcellulose, and a metal salt of carboxymethylcellulose.

Embodiment I-20. The delayed burst release oral formulation of a low-dose naltrexone (LDN) of Embodiment I-6, wherein the water insoluble polymer is talc, microcrystalline cellulose or a combination thereof.

Embodiment I-21. The delayed burst release oral formulation of a low-dose naltrexone (LDN) of Embodiment I-6, wherein the core further comprises at least one disintegrant, wherein the disintegrant is selected from the group consisting of cross-linked polyvinylpyrrolidone, sodium starch glycolate, cross-linked sodium carboxymethylcellulose, pregelatinized starch, microcrystalline starch, water insoluble starch, calcium carboxymethylcellulose, low substituted carboxymethylcellulose, low substituted hydroxypropyl cellulose, magnesium aluminum silicate, and combinations thereof.

Embodiment I-22. The delayed burst release oral formulation of a low-dose naltrexone (LDN) of Embodiment I-6, wherein the water-insoluble hydrophobic carrier of the outer coating is selected from the group consisting of: a dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”; an ethylmethacrylate/chlorotrimethyl ammonium ethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”; a dimethylaminoethylmethacrylate/methylmethacrylate and butylmethacrylate copolymer a copolymer based on neutral methacrylic acid esters and dimethylaminoethyl methacrylate esters wherein the polymer is cationic in the presence of acids; an ethylacrylate and methylacrylate/ethylmethacrylate; and a methyl methylacrylate copolymer, the copolymer being a neutral copolymer based on neutral methacrylic acid and acrylic acid esters, ethylcellulose, shellac, and waxes.

Embodiment I-23. The delayed burst release oral formulation of a low-dose naltrexone (LDN) of Embodiment I-22, wherein the water-insoluble hydrophobic carrier is ethylcellulose.

Embodiment I-24. The delayed burst release oral formulation for localized release of a low-dose naltrexone (LDN) of Embodiment I-6, wherein the water insoluble hydrophilic particular matter of the outer coating is selected from the group consisting of a water insoluble polysaccharide, a water insoluble cross-linked polysaccharide, a water insoluble polysaccharide metal salt including calcium pectinate, a water insoluble cross-linked protein, a water insoluble cross-linked peptide, water insoluble cross-linked gelatin, water insoluble cross-linked hydrolyzed gelatin, water insoluble cross-linked collagen, a water insoluble cross linked polyacrylic acid, a water insoluble cross-linked cellulose derivative, water insoluble cross-linked polyvinyl pyrrolidone, microcrystalline cellulose, insoluble starch, microcrystalline starch and any combination thereof.

Embodiment I-25. The delayed burst release oral formulation of a low-dose naltrexone (LDN) of Embodiment I-24, wherein the water insoluble hydrophilic particular matter is microcrystalline cellulose.

Embodiment I-26. The delayed burst release oral formulation of a low-dose naltrexone (LDN) of Embodiment I-6, wherein the delayed burst oral formulation comprises an enteric coating disposed over the outer coating.

Embodiment I-27. The delayed burst release oral formulation of a low-dose naltrexone (LDN) of Embodiment I-26, wherein the enteric coating is selected from the group consisting of hydroxypropyl methylcellulose phthalate, polyvinyl acetate phthalate, cellulose acetate phthalate, hydroxy propyl methyl cellulose acetate succinate, poly(methacrylic acid, methyl methacrylate)1:1 (EUDRAGIT® L 100), poly(methacrylic acid, ethyl acrylate)1:1 (EUDRAGIT® L 30 D-55), alginic acid and sodium alginate.

Embodiment I-28. The delayed burst release oral formulation of a low-dose naltrexone (LDN) of Embodiment I-27, wherein the enteric coating comprises a methacrylic acid copolymer.

### Embodiments II

Embodiment II-1. An oral delayed burst formulation in a capsule or tablet dosage form comprising naltrexone granules, wherein each naltrexone granule comprises (a) a core comprising naltrexone HCl 1 mg to 4.5 mg per capsule and pharmaceutical excipients; and (b) an outer enteric delayed release coating comprising pH dependent enteric coating polymers.

Embodiment II-2. The oral delayed burst formulation of Embodiment II-1, wherein the formulation is in the form of a tablet.

Embodiment II-3. The oral delayed burst formulation of Embodiment II-1, wherein the core further comprises at least one disintegrant, wherein the disintegrant is selected from the group consisting of cross-linked polyvinylpyrrolidone, sodium starch glycolate, cross-linked sodium carboxymethylcellulose, pregelatinized starch, microcrystalline starch, water insoluble starch, calcium carboxymethylcellulose, low substituted carboxymethylcellulose, low substituted hydroxypropyl cellulose, magnesium aluminum silicate, and combinations thereof.

Embodiment II-4. The oral delayed burst formulation of Embodiment II-1, wherein an enteric delayed-release coating releases a low-dose naltrexone mixture with a lag time of one to three hours after direct contact with a body fluid.

Embodiment II-5. The oral delayed burst formulation of Embodiment II-1, further comprising (c) a subcoat surrounding the core, wherein the subcoat comprises at least one water soluble hydrophilic carrier.

Embodiment II-6. The oral delayed burst formulation of Embodiment II-5, wherein the hydrophilic carrier of the subcoat is selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose HPMC, carboxy methyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, water soluble gum, polysaccharide, hydroxypropyl methylcellulose phthalate, polyvinyl acetate phthalate, cellulose acetate phthalate, hydroxypropyl methylcellulose acetate succinate, poly(methacrylic acid, methyl methacrylate)1:1 and poly(methacrylic acid, ethyl acrylate)1:1, alginic acid, and sodium alginate.

Embodiment II-7. The oral delayed burst formulation of Embodiment II-5, wherein the subcoat further comprises at least one water insoluble particulate matter, wherein the water insoluble particulate matter is selected from the group consisting of microcrystalline cellulose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide and cross-linked polyacrylic acid.

Embodiment II-8. A method for treating a subject for fibromyalgia by orally administering, shortly before sleeping, a with low-dose naltrexone oral delayed burst formulation comprising granules, wherein each granule comprises (a) a core comprising naltrexone HCl (1 mg to 4.5 mg) and pharmaceutical excipients; and (b) an outer enteric delayed release coating comprising pH dependent enteric coating polymers.

Embodiment II-9. The method of Embodiment II-8, wherein the formulation is in the form of a tablet.

Embodiment II-10. The method of Embodiment II-8, wherein the core further comprises at least one disintegrant, wherein the disintegrant is selected from the group consisting of cross-linked polyvinylpyrrolidone, sodium starch glycolate, cross-linked sodium carboxymethylcellulose, pregelatinized starch, microcrystalline starch, water insoluble starch, calcium carboxymethylcellulose, low substituted carboxymethylcellulose, low substituted hydroxypropyl cellulose, magnesium aluminum silicate, and combinations thereof.

Embodiment II-11. The method of Embodiment II-8, wherein an enteric delayed-release coating releases a low-dose naltrexone mixture with a lag time of one to three hours after direct contact with a body fluid.

Embodiment II-12. The method of Embodiment II-8, further comprising (c) a subcoat surrounding the core, wherein the subcoat comprises at least one water soluble hydrophilic carrier.

Embodiment II-13. The method of Embodiment II-12, wherein the hydrophilic carrier of the subcoat is selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose HPMC, carboxy methyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, water soluble gum, polysaccharide, hydroxypropyl methylcellulose phthalate, polyvinyl acetate phthalate, cellulose acetate phthalate, hydroxypropyl methylcellulose acetate succinate, poly(methacrylic acid, methyl methacrylate)1:1 and poly(methacrylic acid, ethyl acrylate)1:1, alginic acid, and sodium alginate.

Embodiment II-14. The method of Embodiment II-12, wherein the subcoat further comprises at least one water insoluble particulate matter, wherein the water insoluble particulate matter is selected from the group consisting of microcrystalline cellulose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide and cross-linked polyacrylic acid.

Embodiment II-15. An oral delayed burst formulation comprising granules, wherein each granule comprises (a) a core comprising from about 10 mg to about 40 mg naloxone and pharmaceutical excipients; and (b) an outer enteric delayed release coating comprising pH dependent enteric coating polymers.

Embodiment II-16. The oral delayed burst formulation comprising granules of Embodiment II-15, further comprising (c) an optional subcoat surrounding the core, wherein the subcoat comprises at least one water soluble hydrophilic carrier.

Embodiment II-17. The oral delayed burst formulation comprising granules of Embodiment II-16, wherein the hydrophilic carrier of the subcoat is selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose HPMC, carboxy methyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, water soluble gum, polysaccharide, hydroxypropyl methylcellulose phthalate, polyvinyl acetate phthalate, cellulose acetate phthalate, hydroxypropyl methylcellulose acetate succinate, poly(methacrylic acid, methyl methacrylate)1:1 and poly(methacrylic acid, ethyl acrylate)1:1, alginic acid, and sodium alginate.

Embodiment II-18. The oral delayed burst formulation comprising granules of Embodiment II-17, wherein the hydrophilic carrier is polyvinyl pyrrolidone.

Embodiment II-19. The oral delayed burst formulation comprising granules of Embodiment II-16, wherein the subcoat further comprises at least one water insoluble particulate matter, wherein the water insoluble particulate matter is selected from the group consisting of microcrystalline cellulose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide and cross-linked polyacrylic acid.

Embodiment II-20. The oral delayed burst formulation comprising granules of Embodiment II-19, wherein the water insoluble particulate matter is talc.

Embodiment II-21. The oral delayed burst, formulation comprising granules of Embodiment II-19, wherein the hydrophilic carrier of the subcoat is a combination of povidone and microcrystalline cellulose.

### Embodiments III

Embodiment III-1. An oral delayed burst formulation comprising
(a) a core comprising naltrexone, or a pharmaceutically acceptable salt thereof, and at least one pharmaceutical excipient, and
(b) a delayed release layer, and
wherein the oral delayed burst formulation comprises between about 1 to about 5 mg of naltrexone, or a corresponding amount of a pharmaceutically acceptable salt, thereof.

Embodiment III-2, The oral delayed burst formulation of Embodiment III-1, wherein the core comprises about 1 to about 10, about 1 to about 8, about 1 to about 5, about 1 to about 4, about 1 to about 3, about 1 to about 2.8, about 1 to about 2.5, about 1 to about 2, about 2 to about 10, about 2 to about 8, about 2 to about 5, about 2 to about 4, about 2 to about 3, about 2 to about 2.8, or about 2 to about 2.5 wt % naltrexone, or a corresponding amount of the pharmaceutically acceptable salt thereof relative to the total weight of the core.

Embodiment III-3, The oral delayed burst formulation of Embodiment III-1 or III-2, wherein the core comprises about 1 to about 5 wt % naltrexone, or a corresponding amount of the pharmaceutically acceptable salt thereof relative to the total weight of the core.

Embodiment III-4. The oral delayed burst formulation of any one of Embodiments III-1 to III-3, wherein the core comprises about 2, about 2.1, about 2.2, about 2.3, about 2.4, about 2.5, about 2.6, about 2.7, about 2.8, about 2.9, about 3, about 4, or about 5 wt % naltrexone, or a corresponding amount of the pharmaceutically acceptable salt thereof relative to the total weight of the core.

Embodiment III-5. The oral delayed burst formulation of any one of Embodiments III-1 to III-4, wherein the core comprises about 2.5 wt % naltrexone, or a corresponding amount of the pharmaceutically acceptable salt thereof relative to the total weight of the core.

Embodiment III-6. The oral delayed burst formulation of any one of Embodiments III-1 to III-5, wherein the core further comprises at least one core disintegrant, wherein the core disintegrant is selected from the group consisting of polyvinylpyrrolidone, starch glycolate, starch, carboxymethylcellulose, hydroxypropylcellulose, magnesium aluminum silicate, and combinations of the foregoing.

Embodiment III-7. The oral delayed burst formulation of Embodiment III-6, wherein the core disintegrant is selected from the group consisting of cross-linked polyvinylpyrrolidone, sodium starch glycolate, cross-linked sodium carboxymethylcellulose, pregelatinized starch, microcrystalline starch, water insoluble starch, calcium carboxymethylcellulose, low substituted carboxymethylcellulose, low substituted hydroxypropylcellulose, magnesium aluminum silicate, and combinations of the foregoing.

Embodiment III-8. The oral delayed burst formulation of Embodiment III-6 or III-7, wherein the core disintegrant is cross-linked sodium carboxymethylcellulose.

Embodiment III-9. The oral delayed burst formulation of any one of Embodiments III-6 to III-8, wherein the core comprises about 0.5 to about 5, about 0.5 to about 4, about 0.5 to about 3, about 0.5 to about 2, about 0.5 to about 1.5, about 0.5 to about 1.3, about 0.5 to about 1, about 0.8 to about 5, about 0.8 to about 4, about 0.8 to about 3, about 0.8 to about 2, about 0.8 to about 1.5, about 0.8 to about 1.3, about 0.8 to about 1, about 1 to about 5, about 1 to about 4, about 1 to about 3, about 1 to about 2, about 1 to about 1.5, or about 1 to about 1.3 wt % of the core disintegrant relative to the total weight of the core.

Embodiment III-10. The oral delayed burst formulation of any one of Embodiments III-6 to III-9, wherein the core comprises about 0.5 to about 3 wt % of the core disintegrant relative to the total weight of the core.

Embodiment III-11, The oral delayed burst formulation of any one of Embodiments III-6 to III-10, wherein the core comprises about 1, about 1.1, about 1.2, about 1.3, about 1.4, or about 1.5 wt % of the core disintegrant relative to the total weight of the core.

Embodiment III-12. The oral delayed burst formulation of any one of Embodiments III-6 to III-11, wherein the core comprises about 1 wt % of the core disintegrant relative to the total weight of the core.

Embodiment III-13, The oral delayed burst formulation of any one of Embodiments III-1 to III-12, wherein the core further comprises a core filler selected from the group consisting of microcrystalline cellulose, starch, lactitol, lactose, inorganic calcium salt, sucrose, and combinations of the foregoing.

Embodiment III-14. The oral delayed burst formulation of Embodiment III-13, wherein the core filler is sucrose.

Embodiment III-15. The oral delayed burst formulation of Embodiment III-13 or III-14, wherein the core comprises about 80 to about 99, about 80 to about 97, about 80 to about 96, about 80 to about 95, about 80 to about 90, about 85 to about 99, about 85 to about 97, about 85 to about 96, about 85 to about 95, about 85 to about 90, about 90 to about 99, about 90 to about 97, about 90 to about 96, about 90 to about 95, about 93 to about 99, about 93 to about 97, about 93 to about 96, or about 93 to about 95 wt % of the core filler relative to the total weight of the core.

Embodiment III-16. The oral delayed burst formulation of any one of Embodiments III-13 to III-15, wherein the core comprises about 90 to about 99 wt % of the core filler relative to the total weight of the core.

Embodiment III-17. The oral delayed burst formulation of any one of Embodiments III-13 to III-16, wherein the core comprises about 90, about 95, about 96, or about 97 wt % of the core filler relative to the total weight of the core.

Embodiment III-18. The oral delayed burst formulation of any one of Embodiments III-13 to III-17, wherein the core comprises about 96 wt % of the core filler relative to the total weight of the core.

Embodiment III-19. The oral delayed burst formulation of any one of Embodiments III-13 to III-18, wherein the naltrexone or pharmaceutically acceptable salt thereof is in a layer in contact with the core filler.

Embodiment III-20, The oral delayed burst formulation of any one of Embodiments III-1 to III-19, wherein the core further comprises a hydrophilic core excipient selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose, carboxymethyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, polysaccharide, hydroxypropyl methylcellulose phthalate, polyvinyl acetate phthalate, cellulose acetate phthalate, hydroxypropyl methylcellulose acetate succinate, poly(methacrylic acid, methyl methacrylate) copolymer, poly(methacrylic acid, ethyl acrylate) copolymer, alginic acid, sodium alginate, and combinations of the foregoing.

Embodiment III-21, The oral delayed burst formulation of Embodiment ill-20, wherein the hydrophilic core excipient is hydroxypropyl methylcellulose.

Embodiment III-22. The oral delayed burst formulation of Embodiment III-20 or III-21, wherein the core comprises about 0.1 to about 1, about 0.1 to about 0.8, about 0.1 to about 0.6, about 0.1 to about 0.5, about 0.1 to about 0.4, about 0.1 to about 0.3, about 0.1 to about 0.25, about 0.1 to about 0.2, about 0.15 to about 1, about 0.15 to about 0.8, about 0.15 to about 0.6, about 0.15 to about 0.5 about 0.15 to about 0.4, about 0.15 to about 0.3, about 0.15 to about 0.25, about 0.15 to about 0.2, about 0.2 to about 1, about 0.2 to about 0.8, about 0.2 to about 0.6, about 0.2 to about 0.5, about 0.2 to about 0.4, about 0.2 to about 0.3, about 0.2 to about 0.25, about 0.25 to about 1, about 0.25 to about 0.8, about 0.25 to about 0.6, about 0.25 to about 0.5, about 0.25 to about 0.4, or about 0.25 to about 0.3 wt % of the hydrophilic core excipient relative to the total weight of the core.

Embodiment III-23. The oral delayed burst formulation of any one of Embodiments III-20 to III-22, wherein the core comprises about 0.1 to about 0.5 wt % of the hydrophilic core excipient relative to the total weight of the core.

Embodiment III-24. The oral delayed burst formulation of any one of Embodiments III-20 to III-23, wherein the core comprises about 0.1, about 1.5, about 0.2, about 0.25, about 0.3, about 3.5, about 0.4, about 0.45, or about 0.5 wt % of the hydrophilic core excipient relative to the total weight of the core.

Embodiment III-25. The oral delayed burst formulation of any one of Embodiments III-20 to III-24, wherein the core comprises about 0.2 wt % of the hydrophilic core excipient relative to the total weight of the core.

Embodiment III-26. The oral delayed burst formulation of any one of Embodiments III-1 to III-25, wherein the core further comprises at least one water insoluble core excipient, wherein the water insoluble excipient is selected from the group consisting of microcrystalline cellulose, lactose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide, cross-linked polyacrylic acid, and combinations of the foregoing.

Embodiment III-27. The oral delayed burst formulation of Embodiment III-26, wherein the water insoluble core excipient is talc.

Embodiment III-28. The oral delayed burst formulation of Embodiment III-26 or III-27, wherein the core comprises about 0.1 to about 1, about 0.1 to about 0.8, about 0.1 to about 0.6, about 0.1 to about 0.5, about 0.1 to about 0.4, about 0.1 to about 0.3, about 0.1 to about 0.25, about 0.1 to about 0.2, about 0.15 to about 1, about 0.15 to about 0.8, about 0.15 to about 0.6, about 0.15 to about 0.5 about 0.15 to about 0.4, about 0.15 to about 0.3, about 0.15 to about 0.25, about 0.15 to about 0.2, about 0.2 to about 1, about 0.2 to about 0.8, about 0.2 to about 0.6, about 0.2 to about 0.5, about 0.2 to about 0.4, about 0.2 to about 0.3, about 0.2 to about 0.25, about 0.25 to about 1, about 0.25 to about 0.8, about 0.25 to about 0.6, about 0.25 to about 0.5, about 0.25 to about 0.4, or about 0.25 to about 0.3 wt % of the water insoluble core excipient relative to the total weight of the core.

Embodiment III-29. The oral delayed burst formulation of any one of Embodiments III-26 to III-28, wherein the core comprises about 0.1 to about 0.5 wt % of the water insoluble core excipient relative to the total weight of the core.

Embodiment III-30. The oral delayed burst formulation of any one of Embodiments III-26 to III-29, wherein the core comprises about 0.1, about 0.15, about 0.2, about 0.25, about 0.3, about 0.35, about 0.4, or about 0.5 wt % of the water insoluble core excipient relative to the total weight of the core.

Embodiment III-31. The oral delayed burst formulation of any one of Embodiments III-26 to III-30, wherein the core comprises about 0.2 wt % of the water insoluble core excipient relative to the total weight of the core.

Embodiment III-32. The oral delayed burst formulation of any one of Embodiments III-1 to III-31, wherein the core comprises cross-linked sodium carboxymethylcellulose, sucrose, and the naltrexone or the pharmaceutically acceptable salt thereof.

Embodiment III-33. The oral delayed burst formulation of any one of Embodiments III-1 to III-32, wherein the core comprises about 1 wt % sodium carboxymethylcellulose, about 95 wt % sucrose, and about 2.5 wt % naltrexone or a corresponding amount of the pharmaceutically acceptable salt thereof relative to the total weight of the core.

Embodiment III-34, The oral delayed burst formulation of Embodiment III-32 or III-33, wherein the core comprises hydroxypropyl methylcellulose and talc.

Embodiment III-35. The oral delayed burst formulation of Embodiment III-34, wherein the core comprises about 0.2% hydroxypropyl methylcellulose and about 0.2 wt % talc relative to the total weight of the core.

Embodiment III-36. The oral delayed burst formulation of any one of Embodiments III-1 to III-35, further comprising (c) a subcoat layer, wherein the subcoat layer comprises at least one hydrophilic subcoat excipient.

Embodiment III-37. The oral delayed burst formulation of Embodiment III-36, wherein the subcoat layer is between the core and the delayed release layer.

Embodiment III-38. The oral delayed burst formulation of Embodiment III-36 or III-37, wherein the oral delayed burst formulation comprises about 90 to about 99, about 90 to about 98, about 90 to about 97, about 90 to about 96.5, about 90 to about 96, about 90 to about 95, about 92 to about 99, about 92 to about 98, about 92 to about 97, about 92 to about 96.5, about 92 to about 96, about 92 to about 95, about 94 to about 99, about 94 to about 98, about 94 to about 97, about 94 to about 96.5, about 94 to about 96, about 94 to about 95, about 95 to about 99, about 95 to about 98, about 95 to about 97, about 95 to about 96.5, about 95 to about 96, about 96 to about 97, about 96 to about 96.6, or about 96 to about 96.5 wt % of the core relative to the total weight of the core and the subcoat layer.

Embodiment III-39. The oral delayed burst formulation of any one of Embodiments III-36 to III-38, wherein the oral delayed burst formulation comprises about 95 to about 99 wt % of the core relative to the total weight of the core and the subcoat layer.

Embodiment III-40. The oral delayed burst formulation of any one of Embodiments III-36 to III-39, wherein the oral delayed burst formulation comprises about 95, about 96, about 96.5, about 96.6, or about 97 wt % of the core relative to the total weight of the core and the subcoat layer.

Embodiment III-41. The oral delayed burst formulation of any one of Embodiments III-36 to III-40, wherein the oral delayed burst formulation comprises about 97 wt % of the core relative to the total weight of the core and the subcoat layer.

Embodiment III-42. The oral delayed burst formulation of any one of Embodiments III-36 to III-41, wherein the hydrophilic subcoat excipient is selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose, carboxymethyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, polysaccharide, hydroxypropyl methylcellulose phthalate, polyvinyl acetate phthalate, cellulose acetate phthalate, hydroxypropyl methylcellulose acetate succinate, poly(methacrylic acid, methyl methacrylate) copolymer, poly(methacrylic acid, ethyl acrylate) copolymer, alginic acid, sodium alginate, and combinations of the foregoing.

Embodiment III-43. The oral delayed burst formulation of any one of Embodiments III-36 to III-42, wherein the hydrophilic subcoat excipient is hydroxypropyl methylcellulose.

Embodiment III-44. The oral delayed burst formulation of any one of Embodiments III-36 to III-43, wherein the subcoat layer comprises about 1 to about 10, about 1 to about 8, about 1 to about 6, about 1 to about 5, about 1 to about 4, about 1 to about 3, about 1 to about 2.5, about 1 to about 2, about 1.5 to about 10, about 1.5 to about 8, about 1.5 to about 6, about 1.5 to about 5, about 1.5 to about 4, about 1.5 to about 3, about 1.5 to about 2.5, about 1.5 to about 2, about 2 to about 10, about 2 to about 8, about 2 to about 6, about 2 to about 5, about 2 to about 4, about 2 to about 3, about 2 to about 2.5, about 2.5 to about 1, about 2.5 to about 8, about 2.5 to about 6, about 2.5 to about 5, about 2.5 to about 4, or about 2.5 to about 3 wt % of the hydrophilic subcoat excipient relative to the total weight of the core and the subcoat layer.

Embodiment III-45. The oral delayed burst formulation of any one of Embodiments III-36 to III-44, wherein the subcoat layer comprises about 1 to about 5 wt % of the hydrophilic subcoat excipient relative to the total weight of the core and the subcoat layer.

Embodiment III-46. The oral delayed burst formulation of any one of Embodiments III-36 to III-45, wherein the subcoat layer comprises about 1, about 2, about 2.5, about 3, about 3.5, about 4, about 4.5, or about 5 wt % of the hydrophilic subcoat excipient relative to the total weight of the core and the subcoat layer.

Embodiment III-47. The oral delayed burst formulation of any one of Embodiments III-36 to III-46, wherein the subcoat layer comprises about 3 wt % of the hydrophilic subcoat excipient relative to the total weight of the core and the subcoat layer.

Embodiment III-48. The oral delayed burst formulation of any one of Embodiments III-36 to III-47, wherein the subcoat layer further comprises at least one water insoluble subcoat excipient, wherein the water insoluble subcoat excipient is selected from the group consisting of microcrystalline cellulose, lactose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide, cross-linked polyacrylic acid, and combinations of the foregoing.

Embodiment III-49. The oral delayed burst formulation of Embodiment III-48, wherein the subcoat layer comprises talc.

Embodiment III-50. The oral delayed burst formulation of Embodiment III-48 or III-49, wherein the subcoat layer comprises about 0.5 to about 2, about 0.5 to about 1.5, about 0.5 to about 1, about 0.5 to about 0.9, about 0.5 to about 0.8, about 0.7 to about 2, about 0.7 to about 1.5, about 0.7 to about 1, about 0.7 to about 0.9, about 0.7 to about 0.8, or about 0.8 to about 0.9 wt % of the water insoluble subcoat excipient relative to the total weight of the core and the subcoat layer.

Embodiment III-51. The oral delayed burst formulation of any one of Embodiments III-48 to III-50, wherein the subcoat layer comprises about 0.5 to about 1 wt % of the water insoluble subcoat excipient relative to the total weight of the core and the subcoat layer.

Embodiment III-52. The oral delayed burst formulation of any one of Embodiments III-48 to III-51, wherein the subcoat layer comprises about 0.7, about 0.8, about 0.9 wt %, or about 1 wt % of the water insoluble subcoat excipient relative to the total weight of the core and the subcoat layer.

Embodiment III-53. The oral delayed burst formulation of any one of Embodiments III-48 to III-52, wherein the subcoat layer comprises about 1 wt % of the water insoluble subcoat excipient relative to the total weight of the core and the subcoat layer.

Embodiment III-54. The oral delayed burst formulation of any one of Embodiments III-36 to III-53, wherein the subcoat layer comprises hydroxypropyl methylcellulose and talc.

Embodiment III-55. The oral delayed burst formulation of any one of Embodiments III-36 to III-54, wherein the subcoat layer comprises about 2.5 wt % hydroxypropyl methylcellulose and about 0.8 wt % talc relative to the total weight of the core and the subcoat layer.

Embodiment III-56. The oral delayed burst formulation of any one of Embodiments III-1 to III-55, wherein the oral delayed burst formulation comprises about 20 to about 40, about 20 to about 35, about 20 about 33, about 20 to about 32, about 20 to about 30, about 25 to about 40, about 25 to about 35, about 25 to about 33, about 25 to about 32, about 28 to about 40, about 28 to about 35, about 28 about 33, about 28 to about 32, about 30 to about 40, about 30 to about 35, about 30 about 33, or about 30 to about 32 wt % of the delayed release layer relative to the total weight of the oral delayed burst formulation.

Embodiment III-57. The oral delayed burst formulation of any one of Embodiments III-1 to III-56, wherein the oral delayed burst formulation comprises about 25 to about 35 wt % of the delayed release layer relative to the total weight of the oral delayed burst formulation.

Embodiment III-58. The oral delayed burst formulation of any one of Embodiments III-1 to III-57, wherein the oral delayed burst formulation comprises about 30, about 31, about 32, about 33, about 34, or about 35 wt % of the delayed release layer relative to the total weight of the oral delayed burst formulation.

Embodiment III-59. The oral delayed burst formulation of any one of Embodiments III-1 to III-58, wherein the oral delayed burst formulation comprises about 32 wt % of the delayed release layer relative to the total weight of the oral delayed burst formulation.

Embodiment III-60. The oral delayed burst formulation of any one of Embodiments III-1 to III-59, wherein the delayed release layer further comprises one or more delayed release polymers selected from the group consisting of hydroxypropyl methylcellulose phthalate, polyvinyl acetate phthalate, cellulose acetate phthalate, hydroxypropyl methylcellulose acetate succinate, poly(methacrylic acid, methyl methacrylate)copolymer, poly(methacrylic acid, ethyl acrylate) copolymer, alginic acid, sodium alginate, and combinations of the foregoing.

Embodiment III-6l. The method of Embodiment III-60, wherein the delayed release polymer is poly(methacrylic acid, ethyl acrylate) copolymer.

Embodiment III-62. The method of Embodiment III-60 or III-61, wherein the delayed release polymer is poly(methacrylic acid, ethyl acrylate)1:1 copolymer.

Embodiment III-63. The oral delay burst formulation of any one of Embodiments III-60 to III-62, wherein the delayed release polymer is an aqueous dispersion.

Embodiment III-64. The oral delayed burst formulation of any one of Embodiments III-60 to III-63, wherein the aqueous dispersion comprises sodium lauryl sulfate and polysorbate.

Embodiment III-65. The oral delayed burst formulation of any one of Embodiments III-60 to III-64, wherein the aqueous dispersion comprises about 0.7% sodium lauryl sulfate and 2.3% polysorbate.

Embodiment III-66. The oral delayed burst formulation of any one of Embodiments III-60 to III-65, wherein the delayed release polymer is EUDRAGIT® L 30 D-55.

Embodiment III-67. The oral delayed burst formulation of any one of Embodiments III-60 to III-66, wherein the delayed release layer comprises about 10 to about 30, about 10 to about 25, about 10 to about 23, about 10 to about 22, about 10 to about 20, about 15 to about 30, about 15 to about 25, about 15 to about 23, about 15 to about 22, about 15 to about 20, about 18 to about 30, about 18 to about 25, about 18 to about 23, about 18 to about 22, about 18 to about 20, about 20 to about 30, about 20 to about 25, about 20 to about 23, or about 20 to about 22 wt % of the delayed release polymer relative to the total weight of the oral delayed burst formulation.

Embodiment III-68. The oral delayed burst formulation of any one of Embodiments III-60 to III-67, wherein the delayed release layer comprises about 15 to about 25 wt % of the delayed release polymer relative to the total weight of the oral delayed burst formulation.

Embodiment III-69. The oral delayed burst formulation of any one of Embodiments III-60 to III-68, wherein the delayed release layer comprises about 15, about 20, or about 25 wt % of the delayed release polymer relative to the total weight of the oral delayed burst formulation.

Embodiment III-70. The oral delayed burst formulation of any one of Embodiments III-60 to III-69, wherein the delayed release layer comprises about 20 wt % of the delayed release polymer relative to the total weight of the oral delayed burst formulation.

Embodiment III-71, The oral delayed burst formulation of any one of Embodiments III-1 to III-70, wherein the delayed release layer further comprises at least one water insoluble delayed release layer excipient, wherein the water insoluble delayed release layer excipient is selected from the group consisting of microcrystalline cellulose, lactose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide, cross-linked polyacrylic acid, and combinations of the foregoing.

Embodiment III-72. The oral delayed burst formulation of Embodiment III-71, wherein the water insoluble delayed release layer excipient is talc.

Embodiment III-73. The oral delayed burst formulation of Embodiment III-71 or III-72, wherein the delayed release layer comprises about 1 to about 20, about 1 to about 15, about 1 to about 12, about 1 to about 10, about 5 to about 20, about 5 to about 15, about 5 to about 12, about 5 to about 10, about 8 to about 20, about 8 to about 15, about 8 to about 12, or about 8 to about 10 wt % of the water insoluble delayed release layer excipient relative to the total weight of the oral delayed burst formulation.

Embodiment III-74. The oral delayed burst formulation of any one of Embodiments III-71 to III-73, wherein the delayed release layer comprises about 5 to about 15 wt % of the water insoluble delayed release layer excipient relative to the total weight of the oral delayed burst formulation.

Embodiment III-75. The oral delayed burst formulation of any one of Embodiments III-71 to III-74, wherein the delayed release layer comprises about 5, about 8, about 10, about 12, about 15, or about 20 wt % of the water insoluble delayed release layer excipient relative to the total weight of the oral delayed burst formulation.

Embodiment III-76. The oral delayed burst formulation of any one of Embodiments III-71 to III-75, wherein the delayed release layer comprises about 10 wt % of the water insoluble delayed release layer excipient relative to the total weight of the oral delayed burst formulation.

Embodiment III-77. The oral delayed burst formulation of any one of Embodiments III-1 to III-76, wherein the delayed release layer further comprises one or more delayed release layer plasticizers selected from the group consisting of triethyl citrate, acetyl triethyl citrate, acetyltributyl citrate, polyethylene glycol acetylated monoglycerides, glycerin, triacetin, propylene glycol, phthalate esters, titanium dioxide, ferric oxides, castor oil, sorbitol, dibutyl sebacate, and combinations of the foregoing.

Embodiment III-78. The oral delayed burst formulation of Embodiment III-77, wherein the delayed release layer comprises triethyl citrate.

Embodiment III-79. The oral delayed burst formulation of Embodiment III-77 or III-78, wherein the delayed release layer comprises about 0.2 to about 10, about 0.2 to about 5, about 0.2 to about 4, about 0.2 to about 3, about 0.2 to about 2.5, about 0.2 to about 2, about 1 to about 10, about 1 to about 5, about 1 to about 4, about 1 to about 3, about 1 to about 2.5, about 1 to about 2, about 1.5 to about 10, about 1.5 to about 5, about 1.5 to about 4, about 1.5 to about 3, about 1.5 to about 2.5, or about 1.5 to about 2 wt % of the delayed release layer plasticizer relative to the total weight of the oral delayed burst formulation.

Embodiment III-80. The oral delayed burst formulation of any one of Embodiments III-77 to III-79, wherein the delayed release layer comprises about 1 to about 5 wt % of the delayed release layer plasticizer relative to the total weight of the oral delayed burst formulation.

Embodiment III-81. The oral delayed burst formulation of any one of Embodiments III-77 to III-80, wherein the delayed release layer comprises about 1, about 1.5, about 2, or about 2.5 wt % of delayed release layer plasticizer relative to the total weight of the oral delayed burst formulation.

Embodiment III-82. The oral delayed burst formulation of any one of Embodiments III-77 to III-81, wherein the delayed release layer comprises about 2 wt % of the delayed release layer plasticizer relative to the total weight of the oral delayed burst formulation.

Embodiment III-83. The oral delayed burst formulation of any one of Embodiments III-77 to III-82, wherein the delayed release layer comprises poly(methacrylic acid, ethyl acrylate) copolymer and triethyl citrate.

Embodiment III-84, The oral delayed burst formulation of any one of Embodiments III-77 to III-83, wherein the delayed release layer comprises about 20 wt % poly(methacrylic acid, ethyl acrylate) copolymer and about 2 wt % triethyl citrate relative to the total weight of oral delayed burst formulation.

Embodiment III-85. The oral delayed burst formulation of Embodiment III-84, wherein the delayed release layer further comprises talc.

Embodiment III-86. The oral delayed burst formulation of Embodiment III-84 or III-85, wherein the delayed release layer comprises about 10 wt % talc relative to the total weight of oral delayed burst formulation.

Embodiment III-87. The oral delayed burst formulation of any one of Embodiments III-1 to III-86, wherein the core comprises cross-linked sodium carboxymethylcellulose and the delayed release layer comprises poly(methacrylic acid, ethyl acrylate) copolymer.

Embodiment III-88. The oral delayed burst formulation of any one of Embodiments III-36 to III-87, wherein the core comprises cross-linked sodium carboxymethylcellulose and sucrose, the subcoat comprises hydroxypropyl methylcellulose, and the delayed release layer comprises poly(methacrylic acid, ethyl acrylate) copolymer.

Embodiment III-89. The oral delayed burst formulation of any one of Embodiments III-1 to III-88, wherein the oral delayed burst formulation comprises sucrose, hydroxypropyl methylcellulose, talc, cross-linked sodium carboxymethylcellulose, poly(methacrylic acid, ethyl acrylate) copolymer, and triethyl citrate.

Embodiment III-90, The oral delayed burst formulation of any one of Embodiments III-1 to III-89, wherein the oral delayed burst formulation comprises about 62.9 wt % sucrose, about 1.7 wt % naltrexone or a corresponding amount of the pharmaceutically acceptable salt thereof, about 1.9 wt % hydroxypropyl methylcellulose, about 10.7 wt % talc, about 0.8 wt % cross-linked sodium carboxymethylcellulose, about 20 wt % poly(methacrylic acid, ethyl acrylate) copolymer, and about 2.0 wt % triethyl citrate.

Embodiment III-91. An oral delayed burst formulation comprising
(a) a core comprising about 1 to about 5 mg of naltrexone, or a pharmaceutically acceptable salt thereof, and at least one pharmaceutical excipient,
(b) a subcoat layer, and
(c) a delayed release layer
wherein the oral delayed burst formulation comprises sucrose, hydroxypropyl methylcellulose, talc, cross-linked sodium carboxymethylcellulose, poly(methacrylic acid, ethyl acrylate) copolymer, and triethyl citrate.

Embodiment III-92. The oral delayed burst formulation of Embodiment III-91, wherein the oral delayed burst formulation comprises about 50 to about 70 wt % sucrose, about 0.5 to about 3 wt % hydroxypropyl methylcellulose, about 7 to about 15 wt % talc, about 0.4 to about 1.5 wt % cross-linked sodium carboxymethylcellulose, about 15 to about 25 wt % poly(methacrylic acid, ethyl acrylate) copolymer, and about 1 to about 4 wt % triethyl citrate relative to the total weight of the oral delayed burst formulation.

Embodiment III-93. The oral delayed burst formulation of Embodiment III-91 or III-92, wherein
the core comprises sucrose, hydroxypropyl methylcellulose, talc, and cross-linked sodium carboxymethylcellulose;
the subcoat layer comprises hydroxypropyl methylcellulose, and talc;
the delayed release layer comprises poly(methacrylic acid, ethyl acrylate) copolymer, talc, and triethyl citrate.

Embodiment III-94. The oral delayed burst formulation of any one of Embodiments III-91 to III-93, wherein
the core comprises about 80 to about 97 wt % sucrose, about 0.1 to about 0.4 wt % hydroxypropyl methylcellulose, about 0.1 to about 0.4 wt % talc, and about 0.5 to about 3 wt % cross-linked sodium carboxymethylcellulose relative to the total weight of the core;
the subcoat layer comprises about 1 to about 4 wt % hydroxypropyl methylcellulose, and about 0.3 to about 2 wt % talc relative to the total weight of the core and subcoat layer;
the delayed release layer comprises about 10 to about 30 wt % poly(methacrylic acid, ethyl acrylate) copolymer, about 5 to about 15 wt % talc, and about 0.5 to about 4 wt % triethyl citrate relative to the total weight of the oral delayed burst formulation.

Embodiment III-95. The oral delayed burst formulation of any one of Embodiments III-1 to III-94, wherein the oral delayed burst formulation comprises about 1 to about 5, about 1 to about 4.9, about 1 to about 4.5, about 1 to about 4, about 1 to about 3.5, about 1 to about 3, about 2 to about 5, about 2 to about 4.5, about 2 to about 4, about 2 to about 3.5, about 2 to about 3, about 3 to about 5, about 3 to about 4.9, about 3 to about 4.5, about 3 to about 4, or about 3 to about 3.5 mg of the naltrexone or a corresponding amount of the pharmaceutically acceptable salt thereof.

Embodiment III-96. The oral delayed burst formulation of any one of Embodiments III-1 to III-95, wherein the oral delayed burst formulation comprises about 2 to about 4.5 mg of the naltrexone or a corresponding amount of the pharmaceutically acceptable salt thereof.

Embodiment III-97. The oral delayed burst formulation of any one of Embodiments III-1 to III-96, wherein the oral delayed burst formulation comprises about 2, about 2.5, about 3, about 3.5, about 4, or about 4.5 mg of the naltrexone or a corresponding amount of the pharmaceutically acceptable salt thereof.

Embodiment III-98. The oral delayed burst formulation of any one of Embodiments III-1 to III-97, wherein the oral delayed burst formulation comprises about 2 mg of the naltrexone or a corresponding amount of the pharmaceutically acceptable salt thereof.

Embodiment III-99. The oral delayed burst formulation of any one of Embodiments III-1 to III-97, wherein the oral delayed burst formulation comprises about 4 mg of the naltrexone or a corresponding amount of the pharmaceutically acceptable salt thereof.

Embodiment III-100. The oral delayed burst formulation of any one of Embodiments III-1 to III-99, wherein the pharmaceutically acceptable salt is naltrexone HCl.

Embodiment III-101. A dose of the oral delayed burst formulation of any one of Embodiments III-1 to III-95, wherein the dose comprises about 1 to about 5, about 1 to about 4.9, about 1 to about 4.5, about 1 to about 4, about 1 to about 3.5, about 1 to about 3, about 2 to about 5, about 2 to about 4.5, about 2 to about 4, about 2 to about 3.5, about 2 to about 3, about 3 to about 5, about 3 to about 4.9, about 3 to about 4.5, about 3 to about 4, or about 3 to about 3.5 mg of the naltrexone or a corresponding amount of the pharmaceutically acceptable salt thereof.

Embodiment III-102. The dose of Embodiment III-101, wherein the dose comprises about 2 to about 4.5 mg of the naltrexone or a corresponding amount of the pharmaceutically acceptable salt thereof.

Embodiment III-103. The dose of Embodiment III-101 or III-102, wherein the dose comprises about 2, about 2.5, about 3, about 3.5, about 4, or about 4.5 mg of the naltrexone or a corresponding amount of the pharmaceutically acceptable salt thereof.

Embodiment III-104. The dose of any one of Embodiments III-102 to III-103, wherein the dose comprises about 2 mg of the naltrexone or a corresponding amount of the pharmaceutically acceptable salt thereof.

Embodiment III-105. The dose of any one of Embodiments III-102 to III-103, wherein the dose comprises about 4 mg of the naltrexone or a corresponding amount of the pharmaceutically acceptable salt thereof.

Embodiment III-106. The dose of any one of Embodiments III-101 to III-105 wherein the pharmaceutically acceptable salt is naltrexone HCl.

Embodiment III-107. A capsule or a tablet comprising the oral delayed burst formulation of any one of Embodiments III-1 to III-95 or the dose of Embodiment III-101.

Embodiment III-108. The capsule or tablet of Embodiment III-107, wherein the capsule is a gelatin capsule.

Embodiment III-109. The capsule or tablet of Embodiment III-107 or III-108, wherein the capsule or tablet comprises about 1 to about 5, about 1 to about 4.9, about 1 to about 4.5, about 1 to about 4, about 1 to about 3.5, about 1 to about 3, about 2 to about 5, about 2 to about 4.5, about 2 to about 4, about 2 to about 3.5, about 2 to about 3, about 3 to about 5, about 3 to about 4.9, about 3 to about 4.5, about 3 to about 4, or about 3 to about 3.5 mg of the naltrexone or a corresponding amount of the pharmaceutically acceptable salt thereof.

Embodiment III-110. The capsule or tablet of any one of Embodiments III-107 to III-109, wherein the capsule or tablet comprises about 2 to about 4.5 mg of the naltrexone or a corresponding amount of the pharmaceutically acceptable salt thereof.

Embodiment III-111. The capsule or tablet of any one of Embodiments III-107 to III-110, wherein the capsule or tablet comprises about 2, about 2.5, about 3, about 3.5, about 4, or about 4.5 mg of the naltrexone or a corresponding amount of the pharmaceutically acceptable salt thereof.

Embodiment III-112. The capsule or tablet of any one of Embodiments III-107 to III-111, wherein the capsule or tablet comprises about 2 mg of the naltrexone or a corresponding amount of the pharmaceutically acceptable salt thereof.

Embodiment III-113. The capsule or tablet of any one of Embodiments III-107 to III-111, wherein the capsule or tablet comprises about 4 mg of the naltrexone or a corresponding amount of the pharmaceutically acceptable salt thereof.

Embodiment III-114. The capsule or tablet of any one of Embodiments III-107 to III-113, wherein the pharmaceutically acceptable salt is naltrexone HCl.

Embodiment III-115. The oral delay burst formulation of any one of Embodiments III-1 to III-100, the dose of any one of Embodiments III-101 to III-106, or the capsule or tablet of any one of Embodiments III-107 to III-114, wherein the oral delayed burst formulation, dose, capsule, or tablet releases at most about 1%, about 5%, about 10%, about 15%, or about 20% of the naltrexone or the pharmaceutically acceptable salt thereof in an acid stage as measured by a dissolution test.

Embodiment III-116, The oral delay burst formulation of any one of Embodiments III-1 to III-100 or III-115, the dose of any one of Embodiments III-101 to III-106 or III-115, or the capsule or tablet of any one of Embodiments III-107 to III-115, wherein the oral delayed burst formulation, dose, capsule, or tablet releases at least about 50%, at least about 55%, at least about 60%, at least about 65%, at least about 70%, at least about 75%, at least about 80%, at least about 85%, at least about 90%, at least about 99%, at least about 99.9%, or about 100% of the naltrexone or the pharmaceutically acceptable salt thereof in a buffer stage as measured by a dissolution test.

Embodiment III-117. The oral delay burst formulation of any one of Embodiments III-1 to III-100, III-115 or III-116, the dose of any one of Embodiments III-101 to III-106, III-115 or III-116, or the capsule or tablet of any one of Embodiments III-107 to III-116, wherein the oral delayed burst formulation, dose, capsule, or tablet releases at most about 1%, about 5%, about 10%, about 15%, or about 20% of the naltrexone the pharmaceutically acceptable salt thereof in an acid stage as measured by a dissolution test and releases at least about 50%, at least about 55%, at least about 60%, at least about 65%, at least about 70%, at least about 75%, at least about 80%, at least about 85%, at least about 90%, at least about 99%, at least about 99.9%, or about 100% of the naltrexone or the pharmaceutically acceptable salt thereof in a buffer stage as measured by the dissolution test.

Embodiment III-118. The oral delay burst, formulation, the dose, or the capsule or tablet of any one of Embodiments III-115 to III-117, wherein the dissolution test is carried out in 750 mL of 0.1 N HCl at 37±0.5° C. for the first two hours and in 1000 mL of pH 5.5 buffer solution at 37±0.5° C. for the subsequent 80 minutes, and is performed in a USP Apparatus II (Paddle) with a rotational speed of 50 rpm.

Embodiment III-119. The oral delay burst formulation, the dose, or the capsule or tablet of any one of Embodiments III-115 to III-117, wherein the dissolution test is carried out in 750 mL of 0.1 N HCl at 37±0.5° C. for the first two hours and in 1000 mL of pH 6.8 buffer solution at 37±0.5° C. for the subsequent 80 minutes, and is performed in a USP Apparatus II (Paddle) with a rotational speed of 50 rpm.

Embodiment III-120. The oral delay burst formulation of any one of Embodiments III-1 to III-100 or III-115 to III-119, the dose of any one of Embodiments III-101 to III-106 or III-115 to III-119, or the capsule or tablet of any one of Embodiments III-107 to III-119, wherein the oral delayed burst formulation, the dose, or the capsule or tablet releases at most about 1%, about 5%, about 10%, about 15%, or about 20% of the naltrexone or the pharmaceutically acceptable salt thereof after exposure to 0.1N HCl for 120 minutes.

Embodiment III-121. The oral delay burst formulation of any one of Embodiments III-1 to III-100 or III-115 to III-120, the dose of any one of Embodiments III-101 to III-106 or III-115 to III-120, or the capsule or tablet of any one of Embodiments III-107 to III-120, wherein the oral delayed burst formulation, the dose, or the capsule or tablet releases at most about 10% of the naltrexone or the pharmaceutically acceptable salt thereof after exposure to 0.1N HCl for 120 minutes.

Embodiment III-122. The oral delay burst formulation of any one of Embodiments III-1 to III-100 or III-115 to III-121, the dose of any one of Embodiments III-101 to III-106 or III-115 to III-121, or the capsule or tablet of any one of Embodiments III-107 to III-121, wherein the oral delayed burst formulation, the dose, or the capsule or tablet releases at least about 50%, about 55%, about 60%, about 65%, or about 70% of the naltrexone or the pharmaceutically acceptable salt thereof after exposure to a pH 5.5 solution for 15 minutes.

Embodiment III-123, The oral delay burst formulation of any one of Embodiments III-1 to III-100 or III-115 to III-122, the dose of any one of Embodiments III-101 to III-106 or III-115 to III-122, or the capsule or tablet of any one of Embodiments III-107 to III-122, wherein the oral delayed burst formulation, the dose, or the capsule or tablet releases at least about 75%, about. 80%, about 85%, or about 90% of the naltrexone or the pharmaceutically acceptable salt thereof after exposure to a pH 5.5 solution for 30 minutes.

Embodiment III-124. The oral delay burst formulation of any one of Embodiments III-1 to III-100 or III-115 to III-123, the dose of any one of Embodiments III-101 to III-106 or III-115 to III-123, or the capsule or tablet of any one of Embodiments III-107 to III-123, wherein the oral delayed burst formulation, the dose, or the capsule or tablet releases at least about 80%, about 85%, about 90%, about 95%, about 99%, about 99.9%, or about 100% of the naltrexone or the pharmaceutically acceptable salt thereof after exposure to a pH 5.5 solution for 45 minutes.

Embodiment III-125. The oral delay burst formulation of any one of Embodiments III-1 to III-100 or III-115 to III-124, the dose of any one of Embodiments III-101 to III-106 or III-115 to III-124, or the capsule or tablet of any one of Embodiments III-107 to III-124, wherein the oral delayed burst formulation, the dose, or the capsule or tablet releases at least about 80%, about 85%, about 90%, about 95%, about 99%, about 99.9%, or about 100% of the naltrexone or the pharmaceutically acceptable salt thereof after exposure to a pH 5.5 solution for 80 minutes.

Embodiment III-126. The oral delay burst formulation of any one of Embodiments III-1 to III-100 or III-115 to III-125, the dose of any one of Embodiments III-101 to III-106 or III-115 to III-125, or the capsule or tablet of any one of Embodiments III-107 to III-125, wherein the oral delayed burst formulation, the dose, or the capsule or tablet releases at most about 1%, about 5%, about 10%, about 15%, or about 20% of the naltrexone or the pharmaceutically acceptable salt thereof after exposure to 0.1N HO for 120 minutes, and releases at least about 80%, about 85%, about 90%, about 95%, about 99%, about 99.9%, or about 100% of the naltrexone or the pharmaceutically acceptable salt thereof after exposure to a pH 5.5 solution for 15, 30, 45, or 80 minutes.

Embodiment III-127. The oral delay burst formulation, the dose, or the capsule or table of any one of Embodiments III-122 to III-126, wherein the pH 5.5 solution is a citrate buffer,

Embodiment III-128. The oral delay burst formulation of any one of Embodiments III-1 to III-100 or III-115 to III-127, the dose of any one of Embodiments III-101 to III-106 or III-115 to III-127, or the capsule or tablet of any one of Embodiments III-107 to III-127, wherein the oral delayed burst formulation, the dose, or the capsule or tablet releases at least about 80%, about 85%, about 90%, about 93%, about 95%, about 99%, about 99.9%, or about 100% the naltrexone or the pharmaceutically acceptable salt thereof after exposure to a pH 6.8 solution for 10 minutes.

Embodiment III-129. The oral delay burst formulation of any one of Embodiments III-1 to III-100 or III-115 to III-128, the dose of any one of Embodiments III-101 to III-106 or III-115 to III-128, or the capsule or tablet of any one of Embodiments III-107 to III-128, wherein the oral delayed burst formulation, the dose, or the capsule or tablet releases at least about 80%, about 85%, about 90%, about 93%, about 95%, about 99%, about 99.9%, or about 100% of the naltrexone or the pharmaceutically acceptable salt thereof after exposure to a pH 6.8 solution for 30 minutes.

Embodiment III-130. The oral delay burst formulation of any one of Embodiments III-1 to III-100 or III-115 to III-129, the dose of any one of Embodiments III-101 to III-106 or III-115 to III-129, or the capsule or tablet of any one of Embodiments III-107 to III-129, wherein the oral delayed burst formulation, the dose, or the capsule or tablet releases at least about 80%, about 85%, about 90%, about 93%, about 95%, about 99%, about 99.9%, or about 100% of the naltrexone or the pharmaceutically acceptable salt thereof after exposure to a pH 6.8 solution for 30 minutes.

Embodiment III-131. The oral delay burst formulation of any one of Embodiments III-1 to III-100 or III-115 to III-130, the dose of any one of Embodiments III-101 to III-106 or III-115 to III-130, or the capsule or tablet of any one of Embodiments III-107 to III-130, wherein the oral delayed burst formulation, the dose, or the capsule or tablet releases at least about 80%, about about 90%, about 95%, about 99%, about 99.9%, or about 100% of the naltrexone or the pharmaceutically acceptable salt thereof after exposure to a pH 6.8 solution for 45 minutes.

Embodiment III-132. The oral delay burst formulation of any one of Embodiments III-1 to III-100 or III-115 to III-131, the dose of any one of Embodiments III-101 to III-106 or III-115 to III-131, or the capsule or tablet of any one of Embodiments III-107 to III-131, wherein the oral delayed burst formulation, the dose, or the capsule or tablet releases at most about 1%, about 5%, about 10%, about 15%, or about 20% of the naltrexone or the pharmaceutically acceptable salt thereof after exposure to 0.1N HCl for 120 minutes, and releases at least about 80%, about 85%, about 90%, about 95%, about 99%, about 99.9%, or about 100% of the naltrexone or the pharmaceutically acceptable salt thereof after exposure to a pH 6.8 solution for 10, 20, 30, or 45 minutes.

Embodiment III-133. The oral delay burst formulation, the dose, or the capsule or table of any one of Embodiments III-128 to III-132, wherein the pH 5.8 solution is a phosphate buffer.

Embodiment III-134. The oral delayed burst formulation of any one of Embodiments III-1 to III-100 or III-115 to III-133, the dose of any one of Embodiments III-107 to III-106 or III-115 to III-133, or the capsule or tablet of any one of Embodiments III-107 to III-133, wherein the oral delayed burst formulation, dose, capsule, or tablet releases at least about 75%, about 80%, about 85%, about 90%, about 99%, about 99,9%, or about 100% of the naltrexone or the pharmaceutically acceptable salt thereof about L5 to about 5, about L5 to about 4, about 1.5 to about 3.5, about 1.5 to about 3, about 1.5 to about 2.5, about 2 to about 5, about 2 to about 4, about 2 to about 3.5, about 2 to about 3, about 2 to about 2.5, about 2.5 to about 5, about 2.5 to about 4, about 2.5 to about 3.5, or about 2.5 to about 3 hours after administration to an individual in need thereof.

Embodiment III-135. The oral delayed burst formulation of any one of Embodiments III-1 to III-100 or III-115 to III-134, the dose of any one of Embodiments III-101 to III-106 or III-115 to III-134, or the capsule or tablet of any one of Embodiments III-107 to III-134, wherein the oral delayed burst formulation, dose, capsule, or tablet releases at least about 80% of the naltrexone or the pharmaceutically acceptable salt thereof about 1.5 to about 3 hours after administration to an individual in need thereof.

Embodiment III-136. The oral delay burst formulation of any one of Embodiments III-1 to III-100 or III-115 to III-135, the dose of any one of Embodiments III-101 to III-106 or III-115 to III-135, or the capsule or tablet of any one of Embodiments III-107 to III-135, wherein the oral delayed burst formulation, dose, capsule, or tablet releases at least about 75%, about 80%, about 85%, about 90%, about 99%, about 99.9%, or about 100% of the naltrexone or the pharmaceutically acceptable salt thereof about 1.5, about 2, about 2.5, about 3, about 3.5, or about 4 hours after administration to an individual in need thereof.

Embodiment III-137. The oral delay burst formulation of any one of Embodiments III-1 to III-100 or III-115 to III-136, the dose of any one of Embodiments III-101 to III-106 or III-115 to III-136, or the capsule or tablet of any one of Embodiments III-107 to III-136, wherein the oral delayed burst formulation, dose, capsule, or tablet releases at least about 80% of die naltrexone or the pharmaceutically acceptable salt thereof about 3 hours after administration to an individual in need thereof.

Embodiment III-138. The oral delay burst, formulation of any one of Embodiments III-1 to III-100 or III-115 to III-137, the dose of any one of Embodiments III-101 to III-106 or III-115 to III-137, or the capsule or tablet of any one of Embodiments III-107 to III-137, wherein the oral delayed burst formulation, dose, capsule, or tablet releases at least about 90% of the naltrexone or the pharmaceutically acceptable salt thereof about 3 hours after administration to an individual in need thereof.

Embodiment III-139. The oral delayed burst formulation of any one of Embodiments III-1 to III-100 or III-115 to III-138, the dose of any one of Embodiments III-101 to III-106 or III-115 to III-138, or the capsule or tablet of any one of Embodiments III-107 to III-138, wherein the oral delayed burst formulation, dose, capsule, or tablet releases at most about 10%, about 5%, about 4%, about 3%, about 2%, about 1%, or about 0% of the naltrexone or the pharmaceutically acceptable salt thereof about 0.5 to about 2, about 0.5 to about 1.5, about 0.5 to about 1, about 1 to about 2, or about 1 to about 1.5 hours after administration to an individual in need thereof.

Embodiment III-140. The oral delayed burst formulation of any one of Embodiments III-1 to III-100 or III-115 to III-139, the dose of any one of Embodiments III-101 to III-106 or III-115 to III-139, or the capsule or tablet of any one of Embodiments III-107 to III-139, wherein the oral delayed burst formulation, dose, capsule, or tablet releases at most about 10% of the naltrexone or the pharmaceutically acceptable salt thereof about 0.5 to about 2 hours after administration to an individual in need thereof.

Embodiment III-141. The oral delayed burst formulation of any one of Embodiments III-1 to III-100 or III-115 to III-140, the dose of any one of Embodiments III-101 to III-106 or III-115 to III-140, or the capsule or tablet of any one of Embodiments III-107 to III-140, wherein the oral delayed burst formulation, dose, capsule, or tablet releases at most about 10%, about 5%, about 4%, about 3%, about 2%, about 1%, or about 0% of the naltrexone or the pharmaceutically acceptable salt thereof about 0.5, about 1, about 1.5, or about 2 hours after administration to an individual in need thereof.

Embodiment III-142. The oral delayed burst formulation of any one of Embodiments III-1 to III-100 or III-115 to III-141, the dose of any one of Embodiments III-101 to III-106 or III-115 to III-141, or the capsule or tablet of any one of Embodiments III-107 to III-141, wherein the oral delayed burst formulation, dose, capsule, or tablet releases at most about 10% of the naltrexone or the pharmaceutically acceptable salt thereof about 2 hours after administration to an individual in need thereof.

Embodiment III-143. The oral delayed burst formulation of any one of Embodiments III-1 to III-100 or III-115 to III-142, the dose of any one of Embodiments III-101 to III-106 or III-115 to III-142, or the capsule or tablet of any one of Embodiments III-107 to III-142, wherein the oral delayed burst formulation, dose, capsule, or tablet releases at most about 10%, about 5%, about 4%, about 3%, about 2%, about 1%, or about 0% of the naltrexone or the pharmaceutically acceptable salt thereof about 0.5 to about 2, about 0.5 to about 1.5, about 0.5 to about 1, about 1 to about 2, or about 1 to about 1.5 hours after administration to an individual in need thereof, and
releases at least about 75%, about 80%, about 85%, about 90%, about 99%, about 99.9%, or about 100% of the naltrexone or the pharmaceutically acceptable salt thereof about L5 to about 5, about 1.5 to about 4, about 1.5 to about 3.5, about 1.5 to about 3, about 1.5 to about 2.5, about 2 to about 5, about 2 to about 4, about 2 to about 3.5, about 2 to about 3, about 2 to about 2.5, about 2.5 to about 5, about 2.5 to about 4, about 2.5 to about 3.5, or about 2.5 to about 3 hours after the administration to the individual.

Embodiment III-144. The oral delayed burst formulation of any one of Embodiments III-1 to III-100 or III-115 to III-143, the dose of any one of Embodiments III-101 to III-106 or III-115 to III-143, or the capsule or tablet of any one of Embodiments III-107 to III-143, wherein the oral delayed burst formulation, dose, capsule, or tablet releases at most about 10% of the naltrexone or the pharmaceutically acceptable salt thereof about 1 to about 2 hours after administration to an individual in need thereof, and
releases at least about 80% the naltrexone or the pharmaceutically acceptable salt thereof about 2 to about 3 hours after the administration to the individual.

Embodiment III-145. The oral delayed burst formulation of any one of Embodiments III-1 to III-100 or III-115 to III-144, the dose of any one of Embodiments III-101 to III-106 or III-115 to III-144, or the capsule or tablet of any one of Embodiments III-107 to III-144, wherein the oral delayed burst formulation, dose, capsule, or tablet releases at most about 10%, about 5%, about 4%, about 3%, about 2%, about 1%, or about 0% of the naltrexone or the pharmaceutically acceptable salt thereof about 0.5, about 1, about 1.5, or about 2 hours after administration to an individual in need thereof, and
releases at least about 75%, about 80%, about 85%, about 90%, about 99%, about 99.9%, or about 100% of the naltrexone or the pharmaceutically acceptable salt thereof about 1.5, about 2, about 2.5, about 3, about 3.5, or about 4 hours after the administration to the individual.

Embodiment III-146. The oral delayed burst formulation of any one of Embodiments III-1 to III-100 or III-115 to III-145, the dose of any one of Embodiments III-101 to III-106 or III-115 to III-145, or the capsule or tablet of any one of Embodiments III-107 to III-145, wherein the oral delayed burst formulation, dose, capsule, or tablet releases at most about 10% of the naltrexone or the pharmaceutically acceptable salt thereof about 2 hours after administration to an individual in need thereof, and
releases at least about 90% the naltrexone or the pharmaceutically acceptable salt thereof about 3 hours after the administration to the individual.

Embodiment III-147. A method for treating chronic pain in a subject in need thereof, the method comprising orally administering the oral delayed burst formulation of any one of Embodiments III-1 to III-101 or III-115 to III-146, the dose of any one of Embodiments III-101 to III-106 or III-115 to III-146, or the capsule or tablet of any one of Embodiments III-107 to III-146 to the subject shortly before sleep.

Embodiment III-148. The method of Embodiment III-147, wherein the subject has fibromyalgia, central sensitization syndrome, chronic regional pain syndrome, opioid dependence, endogenous opioid dysregulation, axial lower back pain, multiple sclerosis, Crohn's disease, diabetic neuropathy, long-Covid, or combinations of the foregoing.

Embodiment III-149. A method for treating fibromyalgia in a subject in need thereof, the method comprising orally administering the oral delayed burst formulation of any one of Embodiments III-1 to III-101 or III-115 to III-146, the dose of any one of Embodiments III-101 to III-106 or III-115 to III-146, or the capsule or tablet of any one of Embodiments III-107 to III-146 to the subject shortly before sleep.

Embodiment III-150. A method for treating long-Covid in a subject in need thereof, the method comprising orally administering the oral delayed burst formulation of any one of Embodiments III-1 to III-101 or III-115 to III-146, the dose of any one of Embodiments III-101 to III-106 or III-115 to III-146, or the capsule or tablet of any one of Embodiments III-107 to III-146 to the subject shortly before sleep.

Embodiment III-151. The method of any one of Embodiments III-147 to III-150, wherein the subject previously had Covid-19.

Embodiment III-152. The method of any one of Embodiments III-147 to III-151, wherein the oral delay burst formulation is orally administered less than about 2, about 1.5, about 1, or about 0.5 hours before sleep.

Embodiment III-153. The method of any one of Embodiments III-147 to III-152, wherein the administration of the oral delayed burst formulation results in a reduced frequency or severity of one or more side effects in the subject in need thereof as compared to the one or more side effects from administration of an immediate release form of naltrexone or pharmaceutically acceptable salt thereof.

Embodiment III-154. The method of any one of Embodiments III-147 to III-153, wherein the administration of a single dose of the oral delayed burst formulation results in a reduced frequency or severity of one or more side effects in the subject in need thereof as compared to the one or more side effects from administration of the same dose of an immediate release form of naltrexone or a corresponding amount of a pharmaceutically acceptable salt thereof.

Embodiment III-155. The method of Embodiment III-147 or III-154, wherein the one or more side effects is selected from the list consisting of headache, dizziness, insomnia, anxiety, nausea, vomiting, diarrhea, alanine aminotransferase increase, aspartate aminotransferase increase, joint stiffness, rashes, abnormal creatinine phosphokinase levels, pharyngitis, somnolence, sedation, depression, dry mouth, muscle cramps, nasopharyngitis, lethargy, cerebral arterial aneurysm, convulsions, disturbance in attention, dysgeusia, mental impairment, migraine, ischemic stroke, paresthesia, suicide attempt, ideation, abdominal discomfort, colitis, constipation, gastroesophageal reflux disease, gastrointestinal hemorrhage, hemorrhoids, acute pancreatitis, paralytic ileus, lymphadenopathy including cervical adenitis, increased white blood cell count, cholecystitis, cholelithiasis, chills, joint stiffness, muscle spasms, myalgia, pain in limb, angina pectoris, unstable angina, atrial fibrillation, congestive cardiac failure, coronary artery atherosclerosis, myocardial infarction, palpitations, deep vein thrombosis, chronic obstructive pulmonary disease, dyspnea, pharyngolaryngeal pain, sinus congestion, dehydration, face edema, night sweats, pruritus, sweating, decreased platelet count, conjunctivitis, blurred vision, and combinations of the foregoing.

Embodiment III-156. The method of any one of Embodiments III-147 to III-155, wherein the one or more side effects is selected from the list consisting of headache, dizziness, insomnia, anxiety, nausea, vomiting, diarrhea, alanine aminotransferase increase, aspartate aminotransferase increase, joint stiffness, rashes, abnormal creatinine phosphokinase levels, pharyngitis, somnolence, sedation, depression, dry mouth, muscle cramps, nasopharyngitis, and combinations of the foregoing.

Embodiment III-157. The method of any one of Embodiments III-147 to III-156, wherein the one or more side effects is selected from the list consisting of headache, dizziness, insomnia, anxiety, nausea, vomiting, diarrhea, alanine aminotransferase increase, aspartate aminotransferase increase, joint stiffness, rashes, abnormal creatinine phosphokinase levels, pharyngitis, and combinations of the foregoing.

Embodiment III-158. An oral delayed burst formulation comprising
(a) a core comprising naloxone, or a pharmaceutically acceptable salt thereof, and at least one pharmaceutical excipient, and
(b) a delayed release layer, and
wherein the oral delayed burst formulation comprises between about 10 mg to about 40 mg of naloxone, or a corresponding amount of a pharmaceutically acceptable salt thereof.

1. An oral delayed burst formulation comprising
(a) a core comprising naltrexone, or a pharmaceutically acceptable salt thereof, and at least one pharmaceutical excipient, and
(b) a delayed release layer comprising about 10 to about 30 wt % of one or more delayed release polymers relative to the total weight of the oral delayed burst formulation, and
wherein the oral delayed burst formulation comprises between about 1 to about 5 mg of naltrexone, or a corresponding amount of a pharmaceutically acceptable salt thereof.

2. (canceled)

3. The oral delayed burst formulation of claim 1, wherein the core comprises about 1 to about 5 wt % naltrexone, or a corresponding amount of the pharmaceutically acceptable salt thereof relative to the total weight of the core.

4-5. (canceled)

6. The oral delayed burst formulation of claim 1, wherein the core further comprises at least one core disintegrant, wherein the core disintegrant is selected from the group consisting of polyvinylpyrrolidone, starch glycolate, starch, carboxymethylcellulose, hydroxypropylcellulose, magnesium aluminum silicate, and combinations of the foregoing.

7-9. (canceled)

10. The oral delayed burst formulation of claim 6, wherein the core comprises about 0.5 to about 3 wt % of the core disintegrant relative to the total weight of the core.

11-12. (canceled)

13. The oral delayed burst formulation of claim 1, wherein the core further comprises a core filler selected from the group consisting of microcrystalline cellulose, starch, lactitol, lactose, inorganic calcium salt, sucrose, and combinations of the foregoing.

14-15. (canceled)

16. The oral delayed burst formulation of claim 13, wherein the core comprises about 90 to about 99 wt % of the core filler relative to the total weight of the core.

17-19. (canceled)

20. The oral delayed burst formulation of claim 1, wherein the core further comprises a hydrophilic core excipient selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose, carboxymethyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, polysaccharide, hydroxypropyl methylcellulose phthalate, polyvinyl acetate phthalate, cellulose acetate phthalate, hydroxypropyl methylcellulose acetate succinate, poly(methacrylic acid, methyl methacrylate) copolymer, poly(methacrylic acid, ethyl acrylate) copolymer, alginic acid, sodium alginate, and combinations of the foregoing.

21-22. (canceled)

23. The oral delayed burst formulation of claim 20, wherein the core comprises about 0.1 to about 0.5 wt % of the hydrophilic core excipient relative to the total weight of the core.

24-35. (canceled)

36. The oral delayed burst formulation of claim 1, further comprising (c) a subcoat layer, wherein the subcoat layer comprises at least one hydrophilic subcoat excipient.

37-38. (canceled)

39. The oral delayed burst formulation of claim 36, wherein the oral delayed burst formulation comprises about 95 to about 99 wt % of the core relative to the total weight of the core and the subcoat layer.

40-41. (canceled)

42. The oral delayed burst formulation of claim 36, wherein the hydrophilic subcoat excipient is selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose, carboxymethyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, polysaccharide, hydroxypropyl methylcellulose phthalate, polyvinyl acetate phthalate, cellulose acetate phthalate, hydroxypropyl methylcellulose acetate succinate, poly(methacrylic acid, methyl methacrylate) copolymer, poly(methacrylic acid, ethyl acrylate) copolymer, alginic acid, sodium alginate, and combinations of the foregoing.

43-44. (canceled)

45. The oral delayed burst formulation of claim 36, wherein the subcoat layer comprises about 1 to about 5 wt % of the hydrophilic subcoat excipient relative to the total weight of the core and the subcoat layer.

46-56. (canceled)

57. The oral delayed burst formulation of claim 1, wherein the oral delayed burst formulation comprises about 25 to about 35 wt % of the delayed release layer relative to the total weight of the oral delayed burst formulation.

58-59. (canceled)

60. The oral delayed burst formulation of claim 1, wherein the one or more delayed release polymers are selected from the group consisting of hydroxypropyl methylcellulose phthalate, polyvinyl acetate phthalate, cellulose acetate phthalate, hydroxypropyl methylcellulose acetate succinate, poly(methacrylic acid, methyl methacrylate)copolymer, poly(methacrylic acid, ethyl acrylate) copolymer, alginic acid, sodium alginate, and combinations of the foregoing.

61. The oral delayed burst formulation of claim 60, wherein the delayed release polymer is poly(methacrylic acid, ethyl acrylate) copolymer.

62. The oral delayed burst formulation of claim 60, wherein the delayed release polymer is poly(methacrylic acid, ethyl acrylate)1:1 copolymer.

63-67. (canceled)

68. The oral delayed burst formulation of claim 60, wherein the delayed release layer comprises about 15 to about 25 wt % of the delayed release polymer relative to the total weight of the oral delayed burst formulation.

69-86. (canceled)

87. The oral delayed burst formulation of claim 1, wherein the core comprises cross-linked sodium carboxymethylcellulose and the delayed release layer comprises poly(methacrylic acid, ethyl acrylate) copolymer.

88-90. (canceled)

91. An oral delayed burst formulation comprising
(a) a core comprising about 1 to about 5 mg of naltrexone, or a pharmaceutically acceptable salt thereof, and at least one pharmaceutical excipient,
(b) a subcoat layer, and
(c) a delayed release layer,
wherein the oral delayed burst formulation comprises sucrose, hydroxypropyl methylcellulose, talc, cross-linked sodium carboxymethylcellulose, poly(methacrylic acid, ethyl acrylate) copolymer, and triethyl citrate.

92. The oral delayed burst formulation of claim 91, wherein the oral delayed burst formulation comprises about 50 to about 70 wt % sucrose, about 0.5 to about 3 wt % hydroxypropyl methylcellulose, about 7 to about 15 wt % talc, about 0.4 to about 1.5 wt % cross-linked sodium carboxymethylcellulose, about 15 to about 25 wt % poly(methacrylic acid, ethyl acrylate) copolymer, and about 1 to about 4 wt % triethyl citrate relative to the total weight of the oral delayed burst formulation.

93-96. (canceled)

97. The oral delayed burst formulation of claim 1, wherein the oral delayed burst formulation comprises about 4.5 mg of the naltrexone or a corresponding amount of the pharmaceutically acceptable salt thereof.

98-99. (canceled)

100. The oral delayed burst formulation of claim 1, wherein the pharmaceutically acceptable salt is naltrexone HCl.

101-106. (canceled)

107. A capsule or a tablet comprising the oral delayed burst formulation of claim 1.

108-146. (canceled)

147. A method for treating chronic pain in a subject in need thereof, the method comprising orally administering the oral delayed burst formulation of claim 1 to the subject shortly before sleep.

148. The method of claim 147, wherein the subject has fibromyalgia, central sensitization syndrome, chronic regional pain syndrome, opioid dependence, endogenous opioid dysregulation, axial lower back pain, multiple sclerosis, Crohn's disease, diabetic neuropathy, long-Covid, or combinations of the foregoing.

149. A method for treating fibromyalgia in a subject in need thereof, the method comprising orally administering the oral delayed burst formulation of claim 1 to the subject shortly before sleep.

150-158. (canceled)

159. The oral delayed burst formulation of claim 91, wherein the oral delayed burst formulation comprises about 4.5 mg of the naltrexone or a corresponding amount of the pharmaceutically acceptable salt thereof.

160. The oral delayed burst formulation of claim 159, wherein the pharmaceutically acceptable salt is naltrexone HCl.

161. A capsule or a tablet comprising the oral delayed burst formulation of claim 160.

162. A method for treating chronic pain in a subject in need thereof, the method comprising orally administering the capsule or tablet of claim 161 to the subject shortly before sleep.

163. The method of claim 162, wherein the subject has fibromyalgia, central sensitization syndrome, chronic regional pain syndrome, opioid dependence, endogenous opioid dysregulation, axial lower back pain, multiple sclerosis, Crohn's disease, diabetic neuropathy, long-Covid, or combinations of the foregoing.

164. A method for treating fibromyalgia in a subject in need thereof, the method comprising orally administering the capsule or table of claim 161 to the subject shortly before sleep.
